(19) United States
(12) Patent Application Publication
(10) Pub. No.: US 2026/0345823 A1
(43) Pub. Date: Nov. 5, 2026

(54) SHANK3 GENE THERAPY APPROACHES

(71) Applicants: Massachusetts Institute of Technology, Cambridge, MA (US); The Broad Institute, Inc., Cambridge, MA (US)

(72) Inventors: Guoping Feng, Cambridge, MA (US); Xian Gao, Cambridge, MA (US); Yuan Mei, Cambridge, MA (US); Qiangge Zhang, Cambridge, MA (US); William Menegas, Cambridge, MA (US); Robert Desimone, Cambridge, MA (US)

(73) Assignees: Massachusetts Institute of Technology, Cambridge, MA (US); The Broad Institute, Inc., Cambridge, MA (US)

(21) Appl. No.: 19/378,942

(22) Filed: Nov. 4, 2025

Related U.S. Application Data
(63) Continuation of application No. PCT/US2024/027909, filed on May 5, 2024.
(60) Provisional application No. 63/464,520, filed on May 5, 2023.

(51) Int. Cl. A61K 48/00; A61K 38/17; A61P 25/00; C12N 15/86

(52) U.S. Cl. CPC A61K 48/005; A61K 38/1709; A61K 48/0033; A61P 25/00; C12N 15/86; C12N 2750/14143

(57) ABSTRACT

Aspects of the disclosure relate to non-naturally occurring polynucleotides encoding a Shank3 protein, AAV vectors comprising the polynucleotides, and gene therapy methods.

### CROSS-REFERENCE TO RELATED APPLICATIONS

This application claims priority under 35 U.S.C. §§ 120 and 365(c) to and is a continuation of International PCT Application, PCT/US2024/027909, filed on May 5, 2024, entitled “SHANK3 GENE THERAPY APPROACHES,” which claims the benefit under 35 U.S.C. § 119(e) of U.S. Provisional Application No. 63/464,520, filed May 5, 2023, entitled “SHANK3 GENE THERAPY APPROACHES,” the entire disclosure of each of which is hereby incorporated by reference in its entirety.

### REFERENCE TO AN ELECTRONIC SEQUENCE LISTING

The contents of the electronic sequence listing (B119570185US01-SEQ-VLJ.xml; Size: 82,083 bytes; and Date of Creation: Nov. 4, 2025) are herein incorporated by reference in their entirety.

### FIELD

The present disclosure relates to gene therapy approaches for delivering polynucleotides encoding a Shank3 protein to a subject who has, is suspected of having, or is at risk of having, a neurodevelopmental disorder.

### BACKGROUND

Deletions and/or mutations involving Shank3 account for about 0.5-1% of all autism spectrum disorder (ASD) patients and about 2% ASD patients with intellectual disability (ID). However, there is no effective treatment for ASD and/or ID. Several challenges have arisen to developing pharmacological treatments that could correct the multitude of pathologies associated with ASD and ID.

### SUMMARY

Aspects of the disclosure relate to the development of an effective gene therapy approach for subjects with Shank3 mutations. Aspects of the disclosure relate to methods of treating or ameliorating symptoms of an autism spectrum disorder (ASD) or Phelan-McDermid syndrome or a syndrome associated with a SHANK3 mutation in a primate subject in need thereof, the method comprising administering to the primate subject a therapeutically effective amount of a composition comprising an AAV vector, wherein the AAV vector comprises a) an artificial genome comprising a polynucleotide encoding a transgene comprising a miniShank3 protein comprising an amino acid sequence at least 90% identical to any one of SEQ ID NOs: 17-20 and has SHANK3 activity, wherein the transgene is operably linked to an hSYN promoter comprising SEQ ID NO: 22 and b) a capsid protein, wherein the capsid protein is AAV9 or a variant thereof. In embodiments, the transgene comprises an amino acid sequence of any one of SEQ ID NOs: 17-20.

In some embodiments, the capsid protein or variant thereof crosses the blood-brain barrier.

In embodiments, the primate subject has at about 3 months, about 6 months or about one year after said administering a decreased response to negative stimuli, increased motivation, flexibility, and memory, increased response to reward, decreased cognitive deficit and/or intellectual disability compared to the primate subject before treatment or a similarly situated untreated control primate. In embodiments, the changes are a statistically significant and/or clinically significant improvement relative to the pre-treatment subject, or relative to a subject that has a condition and is untreated, or based upon natural history of the disease.

In embodiments, the primate subject at about 3 months, about 6 months or about one year or about 18 months or about two years or more than two years after said administering has increased resting fMRI connectivity lower in anterior/medial areas or decreased resting fMRI connectivity lower in posterior/lateral (sensory) areas compared to the primate subject before treatment or a similarly situated untreated control primate. In embodiments, the changes are a statistically significant and/or clinically significant improvement relative to the pre-treatment subject or based upon natural history of the disease.

In embodiments, the primate subject is human. In embodiments, the human is an adult human. In embodiments, is human is about 3 years old or about 10 years old.

In embodiments, the administration is IV administration. In embodiments, the administration is directly to the brain. In embodiments, the administration is intracerebroventricular (ICV) administration.

Aspects of the disclosure relate to non-naturally occurring polynucleotides encoding a Shank3 protein, wherein the Shank3 protein comprises an SH3 domain, a PDZ domain, a Homer binding domain, a Cortactin binding domain, and a SAM domain. In some embodiments, the SH3 domain comprises at least 90% identity to residues 474-525 of SEQ ID NO: 6 or at least 90% identity to residues 473-524 of SEQ ID NO: 5. In some embodiments, the PDZ domain comprises at least 90% identity to residues 573-662 of SEQ ID NO: 6 or at least 90% identity to residues 572-661 of SEQ ID NO: 5. In some embodiments, the Homer binding domain comprises at least 90% identity to residues 1294-1323 of SEQ ID NO: 5 or 6. In some embodiments, the Cortactin binding domain comprises at least 90% identity to residues 1400-1426 of SEQ ID NO: 5 or 6 and/or. In some embodiments, the SAM domain comprises at least 90% identity to residues 1664-1729 of SEQ ID NO: 6 or at least 90% identity to residues 1663-1728 of SEQ ID NO:5. In some embodiments, the polynucleotide is less than 4.7 kb.

In some embodiments, the polynucleotide further comprises a proline-rich region. In some embodiments, the SH3 domain comprises residues 474-525 of SEQ ID NO: 6 or residues 473-524 of SEQ ID NO: 5, the PDZ domain comprises residues 573-662 of SEQ ID NO: 6 or residues 572-661 of SEQ ID NO: 5, the Homer binding domain comprises residues 1294-1323 of SEQ ID NO: 5 or 6; the Cortactin binding domain comprises residues 1400-1426 of SEQ ID NO: 5 or 6 and/or the SAM domain comprises residues 1664-1729 of SEQ ID NO: 6 or residues 1663-1728 of SEQ ID NO:5.

In some embodiments, the polynucleotide comprises at least 90% identity to SEQ ID NO: 1 or 2. In some embodiments, the polynucleotide comprises SEQ ID NO: 1 or 2.

In some embodiments, the Shank3 protein encoded by the polynucleotide further comprises an ankyrin repeat domain. In some embodiments, the ankyrin repeat domain comprises at least 90% identity to residues 148-345 of SEQ ID NO: 6 or at least 90% identity to residues 147-313 of SEQ ID NO: 5. In some embodiments, the polynucleotide comprises residues 148-345 of SEQ ID NO: 6 or residues 147-313 of SEQ ID NO: 5.

In some embodiments, the polynucleotide comprises at least 90% identity to SEQ ID NO: 3 or 4. In some embodiments, the polynucleotide comprises SEQ ID NO: 3 or 4.

In some embodiments, the polynucleotide is less than about 4.6 kb, 4.5 kb, 4.4 kb, 4.3 kb, 4.2 kb, 4.1 kb, 4.0 kb, 3.9 kb, 3.8 kb, 3.7 kb, 3.6 kb, 3.5 kb, 3.4 kb, 3.3 kb, 3.2 kb, 3.1 kb, 3.0 kb, 2.9 kb, 2.8 kb, 2.7 kb, 2.6 kb, 2.5 kb, 2.4 kb, 2.3 kb, 2.2 kb, or 2.1 kb.

In some embodiments, the Shank3 protein is less than 65% identical to SEQ ID NO: 5 or 6 over the full length of SEQ ID NO: 5 or 6.

In some embodiments, the Shank3 protein comprises an amino acid sequence that is at least 90% identical to any one of SEQ ID NOs: 17-20. In some embodiments, the Shank3 protein comprises an amino acid sequence comprising any one of SEQ ID NOs: 17-20.

Further aspects of the disclosure relate to Shank3 proteins encoded by polypeptides described herein.

Further aspects of the disclosure relate to vectors comprising polynucleotides described herein. In some embodiments, the vector is a viral vector. In some embodiments, the vector is an AAV vector. In some embodiments, the vector comprises a promoter operably linked to the polynucleotide described herein. In some embodiments, the polynucleotide is flanked by AAV inverted terminal repeat (ITRs). In some embodiments, the AAV vector comprises a sequence that is at least 90% identical to SEQ ID NO: 7 or 21 and encodes a protein with Shank3 activity. In some embodiments, the AAV vector comprises the sequence of SEQ ID NO: 7 or 21 and encodes a protein with Shank3 activity. In some embodiments, the AAV vector comprises a sequence that is at least 90% identical to SEQ ID NO: 2 or 4 and encodes a protein with Shank3 activity. In some embodiments, the AAV vector comprises the sequence of SEQ ID NO: 2 or 4, which encodes a protein with Shank3 activity. In some embodiments, the AAV vector comprises a sequence that is at least 90% identical to SEQ ID NO: 1 or 3 and encodes a protein with Shank3 activity. In some embodiments, the AAV vector comprises the sequence of SEQ ID NO: 1 or 3, which encodes a protein with Shank3 activity.

Further aspects of the disclosure relate to methods of treatment comprising administering a pharmaceutical composition comprising AAV particles comprising an AAV vector and a capsid protein, wherein the capsid is of a serotype selected from AAV1, 2, 5, 6, 8, 9, rh10, and PHP.eB. In some embodiments, the serotype is AAV9 or a variant thereof. In some embodiments, the serotype is AAV9. In some embodiments, the serotype is AAV10. In some embodiments, the serotype is PHP.eB. In some embodiments, the capsid is a capsid that crosses the blood-brain barrier, for example, those found in Goertsen, David et al. “AAV capsid variants with brain-wide transgene expression and decreased liver targeting after intravenous delivery in mouse and marmoset.” Nature neuroscience vol. 25,1 (2022), incorporated by reference in its entirety.

In some embodiments, the AAV vector further comprises a promoter. In some embodiments, the promoter is a human promoter. In some embodiments, the promoter is hSyn1.

Further aspects of the disclosure relate to methods comprising administering AAV vectors or particles described herein to a subject in need thereof. In some embodiments, the subject is a human subject. In some embodiments, the human subject is an adult. In some embodiments, the human subject is not an adult. In some embodiments, the human subject is not older than 25 years old. In some embodiments, the human subject is about 10 years old or younger. In some embodiments, the human subject is 3 years old or younger. In some embodiments, the subject has, is suspected of having, or is at risk of having, a neurodevelopmental disorder. In some embodiments, the subject has, is suspected of having, or is at risk of having, an autism spectrum disorder (ASD). In some embodiments, the subject exhibits one or more symptoms of an ASD. In some embodiments, the subject has, is suspected of having, or is at risk of having, Phelan-McDermid syndrome. In some embodiments, the subject has a mutation in SHANK3. In some embodiments, the subject is SHANK3+/−. In some embodiments, the subject exhibits one or more of: developmental delay, intellectual disability (ID), sleep disturbance, hypotonia, lack of speech, or language delay.

Further aspects of the disclosure relate to methods of treating a subject having a neurodevelopmental disorder. In some embodiments, the disclosure relates to methods of treating a subject having an autism spectrum disorder (ASD). In some embodiments, the disclosure relates to methods of treating a subject having Phelan-McDermid syndrome. In some embodiments, the methods of treatment comprise administering to the subject an effective amount of a composition comprising an AAV vector that comprises a polynucleotide encoding a Shank3 protein. In some embodiments, the composition is in a pharmaceutically acceptable carrier.

In some embodiments, the AAV vector is delivered to the brain of the subject. In some embodiments, the AAV vector is delivered to the cortex, striatum and/or thalamus of the subject.

In some embodiments, the subject has, is suspected of having, or is at risk of having, reduced expression of the Shank3 gene relative to a control subject. In some embodiments, the control subject is a subject that does not have, is not suspected of having, or is not at risk of having, a neurodevelopmental disorder, an autism spectrum disorder (ASD), and/or Phelan-McDermid syndrome. In some embodiments, reduced expression of the Shank3 gene is caused by disruption of at least one copy of the Shank3 gene. In some embodiments, disruption of the Shank3 gene comprises a deletion in at least one copy of the Shank3 gene. In some embodiments, disruption of the Shank3 gene comprises one or more mutations within at least one copy of the Shank3 gene.

Further aspects of the disclosure relate to MiniShank3 proteins comprising a sequence that is at least 80%, at least 85%, at least 90%, or at least 95% identical to any one of SEQ ID NOs: 17-20. In some embodiments, the MiniShank3 protein comprises the sequence of any one of SEQ ID NOs: 17-20.

Aspects of the disclosure relate to methods of treating an autism spectrum disorder (ASD) in a primate subject, the method comprising administering to the primate subject an effective amount of a composition comprising an AAV vector, wherein the AAV vector comprises a polynucleotide encoding a Shank3 protein.

In some embodiments, the composition is administered intravenously. In some embodiments, the composition is delivered to the brain of the primate subject. In some embodiments, the primate subject is 10 years old or younger. In some embodiments, the primate subject is 3 years old or younger. In some embodiments, the Shank3 protein is a miniShank3 protein. In some embodiments, the miniShank3 protein comprises a sequence that is at least 80%, at least 85%, at least 90%, or at least 95% identical to SEQ ID NO: 18 or 20.

### BRIEF DESCRIPTION OF THE DRAWINGS

FIGS. 1A-1E are images and graphs depicting results of behavioral studies involving marmosets administered gene therapy to test the atypical behavior reversal of the gene therapy. Behavioral data was extracted from video in many behavioral settings, demonstrated by the exemplary video collection, trace, and unsupervised behavioral states (e.g., active, social, alone, and climbing) as shown in FIG. 1A. The frequency of time in different behavioral states was correlated in wild type (WT) and Shank3+/− (SH) marmosets relative to the cage average frequency showing that Shank3+/− marmoset behavior is less correlated to cagemates, as shown in the correlation graphs in FIGS. 1B and 1C. Exemplary recordings of four animals and high and low correlations are shown in FIG. 1D. Correlation and average correlation with cagemates across all sessions (i.e., all ages and session types) in wild type (WT), Shank3+/− (SH), and mini-SHANK3 gene therapy-treated (GT) marmosets shows that Shank3+/− marmoset behavior is less correlated to cagemates and gene therapy restores it to WT level, as shown in FIG. 1E.

FIGS. 2A-2D are images and graphs depicting results of touchscreen-based behavioral stimuli studies involving marmosets administered gene therapy to test the atypical behavior reversal of the gene therapy. Exemplary images and results are shown in FIG. 2A. The correlation with cagemates in wild type (WT), Shank3+/− (SH), and mini-SHANK3 gene therapy-treated (GT) marmosets shows that Shank3+/− marmoset touchscreen shape and face behavior is less correlated to cagemates and gene therapy restores it to WT level, as shown in FIGS. 2B and 2C. Similar results in response to touchscreen with reward in wild type (WT), Shank3+/− (SH), and mini-SHANK3 gene therapy-treated (GT) marmosets are shown in FIG. 2D.

FIGS. 3A-3D are images and graphs depicting results of negative valence behavioral stimuli studies involving marmosets administered gene therapy to test the atypical behavior reversal of the gene therapy. Exemplary images and results are shown in FIG. 3A. The correlation with cagemates in wild type (WT), Shank3+/− (SH), and mini-SHANK3 gene therapy-treated (GT) marmosets shows that Shank3+/− marmoset noise and scary mask response behavior is less correlated to cagemates and gene therapy reverses this behavior, as shown in FIGS. 3B and 3C. Similar results in response to mask (negative stimulus), measured by time near mask in all ages in wild type (WT), Shank3+/− (SH), and mini-SHANK3 gene therapy-treated (GT) marmosets are shown in FIG. 3D.

FIGS. 4A-4C are images and graphs depicting results of social behavioral stimuli studies involving marmosets administered gene therapy to test the atypical behavior reversal of the gene therapy. Exemplary images and results are shown in FIG. 4A. The correlation with cagemates in wild type (WT), Shank3+/− (SH), and mini-SHANK3 gene therapy-treated (GT) marmosets shows that Shank3+/− marmoset novel toys and intruder marmoset response behavior is less correlated to cagemates and gene therapy reverses this behavior, as shown in FIGS. 4B and 4C.

FIGS. 5A-5C are images and graphs depicting results of positive valence behavioral stimuli studies involving marmosets administered gene therapy to test the atypical behavior reversal of the gene therapy. Exemplary images and results are shown in FIG. 5A. The correlation with cagemates in wild type (WT), Shank3+/− (SH), and mini-SHANK3 gene therapy-treated (GT) marmosets shows that Shank3 novel object and food treat response behavior is less correlated to cagemates and gene therapy reverses this behavior, as shown in FIGS. 5B and 5C.

FIGS. 6A-6B are images and graphs depicting results of behavioral studies involving marmosets administered gene therapy to test the atypical social behavior reversal of the gene therapy. Exemplary images and results of control and Shank3 are shown in FIG. 6A. The relative interest before pairing (y-axis) and relative velocity when paired (x-axis) was quantified in wild type (WT), Shank3+/− (SH), and mini-SHANK3 gene therapy-treated (GT) marmosets showing that Shank3+/− (SH) animals are less interested in novel animals relative to wild type (WT) animals and gene therapy reverses this behavior, as shown in FIG. 6B.

FIGS. 7A-7F are images and graphs depicting results of cognitive task studies involving marmosets administered gene therapy to test the cognitive deficit reversal of the gene therapy. The cognitive tasks studied test motivation, flexibility, and memory. For example, motivational component of cognitive tasks includes free reward, sucrose preference, and progressive ration. Examples of intellectual component cognitive tasks studied were choice task, association updating, location updating, working memory, and foraging. The percent of correct touches was quantified in control (wild type/WT) and Shank3+/− (SH) mice on Day 1 and Day 2 demonstrating that Shank3+/− (SH) animals have lower accuracy and lower trial number than wild type (WT), as shown in FIGS. 7A and 7B. Exemplary results of fixed position touches in wild type (WT), Shank3+/− (SH), and mini-SHANK3 gene therapy-treated (GT) marmosets are shown in FIG. 7C. The percentage (%) of touches near center in different touch location tasks (i.e., free reward, choice, updating, moving, and working memory) shows that mini-SHANK3 gene therapy-treated (GT) marmoset has more task-related touches across tasks, as shown in FIG. 7D. The number of trials in different touch location tasks (i.e., free reward, choice, updating, moving, and working memory) shows that mini-SHANK3 gene therapy-treated (GT) marmoset have increased number of trials across tasks, as shown in FIG. 7E. The percentage (%) total accuracy in different touch location tasks (i.e., free reward, choice, updating, moving, and working memory) shows that mini-SHANK3 gene therapy-treated (GT) marmoset have improved accuracy in some tasks, as shown in FIG. 7F.

FIGS. 8A-8C are fMRI images and graphs depicting results of studies involving marmosets administered gene therapy to test the abnormal connectivity reversal of the gene therapy. Resting fMRI connectivity measurements in wild type (WT), Shank3+/− (SH), and mini-SHANK3 gene therapy-treated (GT) marmosets, both local and global, show that resting fMRI connectivity in Shank3+/− (SH) marmosets is lower in anterior/medial areas and higher in posterior/lateral (sensory) areas and gene therapy rescues these abnormalities, as shown in FIGS. 8A-8C.

### DETAILED DESCRIPTION

Aspects of the disclosure relate to gene therapy approaches for treating neurodevelopmental disorders. The Examples show that treatment of SHANK3+/− primates with mini-SHANK3 gene therapy restores atypical behavior, atypical social behavior, cognitive deficits, communication deficits, and/or abnormal connectivity to levels at or near that of WT primates. Gene therapy strategies disclosed herein use an AAV system to deliver a functional copy of the Shank3 gene into brain cells to restore cellular function.

SHANK3 encodes synaptic scaffolding proteins at the excitatory glutamatergic synapses, coordinates the recruitment of signaling molecules and orchestrates assembly of the macromolecular postsynaptic protein complex, which is crucial for proper synaptic development and function. Deletion of SHANK3 is a major cause of the core neurodevelopmental and neurobehavioral deficits in Phelan-McDermid syndrome. Human genetic studies also identified SHANK3 mutations as accounting for about 1% of autism spectrum disorder (ASD). Patients with Phelan-McDermid syndrome and other individuals with SHANK3 mutations often exhibit a variety of comorbid traits, which include developmental delay, sleep disturbances, hypotonia, lack of speech or severe language delay, and characteristic features of ASD. Currently, there is no effective treatment for ASD.

The association of ASD with Shank3 provided an immediate link between synaptic dysfunction and the pathophysiology of ASD. Animal models bridge the human genetics of ASD to brain pathology underlying clinical presentation, and ultimately help to discover and evaluate effective therapeutics. Previous studies in flies, fish, and rodents have revealed synaptic dysfunction and behavioral abnormalities due to loss of SHANK3. For example, disruption of Shank3 in mouse models have resulted in synaptic defects, impaired social interactions, motor difficulties, repetitive grooming and increased anxiety level. Since Shank3 deficiency causes severe sleep disturbances in rodents, monkeys and human patients, sleep efficiency provides a unique biomarker for ASD. Furthermore, the Shank3-deficient mouse model presents predictive validity as the synaptic defects and behavioral abnormalities are reversible when Shank3 is restored. Therefore, gene replacement is well suited as a therapeutic strategy for this monogenic disease.

Novel recombinant adeno-associated viruses (rAAVs) represent a promising gene delivery platform because of their wide range of tissue tropism, low immunogenicity, highly efficient and sustained gene transduction, and clinically proven track record in safety. However, it is known in the art that Shank3 is a large protein with a coding sequence of about 5.7 kb, exceeding the packaging capacity of AAV vectors. As disclosed in and incorporated by reference from PCT Publication No. WO2022/040239 and US Patent Publication No. US2023/0340041, certain regions of the Shank3 protein were found not to be critical for the function of the protein, and therefore, heterologous Shank3 expression constructs were designed that have a significantly smaller coding sequence (about 2.1 kb to about 3.1 kb) because they encode for a version of the Shank3 protein that has certain non-essential regions removed. The resulting miniaturized Shank3 proteins can be delivered by vector such as AAVs. A miniaturized Shank3 protein can restore defective functions caused by deletion or mutation of the gene encoding the Shank3 protein in a mouse model, and accordingly, can potentially rescue abnormalities caused by diseases that are associated with Shank3 mutations or deletions. This is in contrast with previous methods known in the art, which focus on repairing or improving partial fragments of the Shank3 protein but which are not able to restore the functions of the Shank protein. Thus, the present disclosure relates to methods and compositions for treating neurodevelopmental disorders by restoring the activity of Shank3 using a miniaturized Shank3 protein (“MiniShank3”).

### Shank Proteins

The Shank family of proteins (e.g., Shank1, Shank 2, and Shank3) are master scaffolding proteins that tether and organize scaffolding proteins at the synapses of excitatory neurons. Members of this family share at least five main domain regions: N-terminal ankyrin repeats, SH3 domain, PDZ domain, proline-rich region, and a C-terminal SAM domain. Through these functional domains, Shank proteins interact with many postsynaptic density (PSD) proteins. Without wishing to be bound by any theory, Shank proteins can bind to SAPAP which in turn binds to PSD95 to form the PSD95/SAPAP/Shank postsynaptic complex. Together, these multidomain proteins are proposed to form a key scaffold, orchestrating the assembly of the macromolecular postsynaptic signaling complex at glutamatergic synapses. This complex has been shown to play important roles in targeting, anchoring, and dynamically regulating synaptic localization of neurotransmitter receptors and signaling molecules. In another example, the Shank family of proteins is connected to the mGluR pathway through its binding to Homer.

Due to its link to actin-binding proteins, Shank also plays a major role in spine development. It has been found that transfection of Shank3 was sufficient to induce functional dendritic spine synapses in cultured aspiny cerebellar granule cells, indicating a role in spine induction. Shank3 has three primary isoforms including Shank3_{α}, the longest Shank3 isoform, Shank3_{β} and Shank3_{γ}. It has been reported that siRNA knockdown of Shank3 reduced the number and increased the length of dendritic spines in DIV18 cultured hippocampal neurons, implicating a role in spine maturation. This proposed function was supported by the finding that overexpression of Shank1 enlarged already present dendritic spines in cultured hippocampal neurons. Furthermore, Shank1 mutant mice have been reported to have smaller dendritic spines and weaker synaptic transmission.

In some embodiments, the present disclosure relates to Shank proteins that are capable of restoring synaptic activity in subjects with disrupted Shank protein activity. In some embodiments, the disrupted Shank protein activity is present in subjects who have neurodevelopmental disorders, an autism spectrum disorder (ASD), and/or Phelan-McDermid syndrome. In some embodiments, the Shank proteins associated with the present disclosure are Shank1 proteins. In some embodiments, the present disclosure relates to expression in a subject in need thereof a polynucleotide encoding Shank1 or a variant of Shank1. In some embodiments, the Shank proteins in the present disclosure are Shank2 proteins. In some embodiments, the present disclosure relates to expression in a subject in need thereof a polynucleotide encoding Shank2 or a variant of Shank2. In some embodiments, the Shank proteins in the present disclosure are Shank3 proteins. In some embodiments, the present disclosure relates to expression in a subject in need thereof a polynucleotide encoding Shank3 or a variant of Shank3. It should be appreciated that Shank proteins associated with the present disclosure can include any Shank protein, including variants or fragments thereof, that function as scaffolding proteins at the synapses of excitatory neurons.

Also disclosed herein are polynucleotides encoding Shank proteins (Shank1, Shank 2, and Shank3) for use in gene therapy.

The Shank3 full length mouse protein sequence corresponding to GenBank Accession

No. BAE16756.1 is provided by SEQ ID NO: 5:
MDGPGASAVVVRVGIPDLQQTKCLRLDPTAPVWAAKQRVLCALNHSLQDALNYGL
FQPPSRGRAGKFLDEERLLQDYPPNLDTPLPYLEFRYKRRVYAQNLIDDKQFAKLHT
KANLKKFMDYVQLHSTDKVARLLDKGLDPNFHDPDSGECPLSLAAQLDNATDLLK
VLRNGGAHLDFRTRDGLTAVHCATRQRNAGALTTLLDLGASPDYKDSRGLTPLYHS
ALGGGDALCCELLLHDHAQLGTTDENGWQEIHQACRFGHVQHLEHLLFYGANMGA
QNASGNTALHICALYNQESCARVLLFRGANKDVRNYNSQTAFQVAIIAGNFELAEVI
KTHKDSDVVPFRETPSYAKRRRLAGPSGLASPRPLQRSASDINLKGDQPAASPGPTLR
SLPHQLLLQRLQEEKDRDRDGELENDISGPSAGRGGHNKISPSGPGGSGPAPGPGPAS
PAPPAPPPRGPKRKLYSAVPGRKFIAVKAHSPQGEGEIPLHRGEAVKVLSIGEGGFWE
GTVKGRTGWFPADCVEEVQMRQYDTRHETREDRTKRLFRHYTVGSYDSLTSHSDY
VIDDKVAILQKRDHEGFGFVLRGAKAETPIEEFTPTPAFPALQYLESVDVEGVAWRA
GLRTGDFLIEVNGVNVVKVGHKQVVGLIRQGGNRLVMKVVSVTRKPEEDGARRRA
PPPPKRAPSTTLTLRSKSMTAELEELASIRRRKGEKLDEILAVAAEPTLRPDIADADSR
AATVKQRPTSRRITPAEISSLFERQGLPGPEKLPGSLRKGIPRTKSVGEDEKLASLLEG
RFPRSTSMQDTVREGRGIPPPPQTAPPPPPAPYYFDSGPPPTFSPPPPPGRAYDTVRSSF
KPGLEARLGAGAAGLYDPSTPLGPLPYPERQKRARSMIILQDSAPEVGDVPRPAPAA
TPPERPKRRPRPSGPDSPYANLGAFSASLFAPSKPQRRKSPLVKQLQVEDAQERAALA
VGSPGPVGGSFAREPSPTHRGPRPGSLDYSSGEGLGLTFGGPSPGPVKERRLEERRRS
TVFLSVGAIEGSPPSADLPSLQPSRSIDERLLGTGATTGRDLLLPSPVSALKPLVGGPSL
GPSGSTFIHPLTGKPLDPSSPLALALAARERALASQTPSRSPTPVHSPDADRPGPLFVD
VQTRDSERGPLASPAFSPRSPAWIPVPARREAEKPPREERKSPEDKKSMILSVLDTSLQ
RPAGLIVVHATSNGQEPSRLGAEEERPGTPELAPAPMQAAAVAEPMPSPRAQPPGSIP
ADPGPGQGSSEEEPELVFAVNLPPAQLSSSDEETREELARIGLVPPPEEFANGILLTTPP
PGPGPLPTTVPSPASGKPSSELPPAPESAADSGVEEADTRSSSDPHLETTSTISTVSSMS
TLSSESGELTDTHTSFADGHTFLLEKPPVPPKPKLKSPLGKGPVTFRDPLLKQSSDSEL
MAQQHHAASTGLASAAGPARPRYLFQRRSKLWGDPVESRGLPGPEDDKPTVISELSS
RLQQLNKDTRSLGEEPVGGLGSLLDPAKKSPIAAARLFSSLGELSTISAQRSPGGPGG
GASYSVRPSGRYPVARRAPSPVKPASLERVEGLGAGVGGAGRPFGLTPPTILKSSSLSI
PHEPKEVRFVVRSVSARSRSPSPSPLPSPSPGSGPSAGPRRPFQQKPLQLWSKFDVGD
WLESIHLGEHRDRFEDHEIEGAHLPALTKEDFVELGVTRVGHRMNIERALRQLDGS

In some embodiments, the Shank3 full length mouse protein sequence corresponding to SEQ ID NO: 5 is encoded by a nucleic acid sequence corresponding to GenBank Accession No. NM_021423, provided by SEQ ID NO: 15:

atggacggccccggggccagcgccgtggtcgtgcgcgtcggcatcccggacctgcaacaaacgaagtgcctgcgtctg
gacccaaccgcgcccgtgtgggccgccaagcagcgtgtgctctgcgccctcaatcatagccttcaagacgcgctcaactacgggcta
ttccagcctccctcccggggtcgcgccggcaagttcctggatgaagagcggctcttacaggactacccgcctaacctggacacgccc
ctgccctatctggagttccgatacaagcggagagtttatgcccagaacctcatagatgacaagcagtttgcaaagctacacacaaagg
caaacctgaagaagttcatggactatgtccagctacacagcacagataaggtggcccgcctgctggacaaggggctggaccccaatt
tccatgaccctgactcaggagagtgccctctgagccttgcggcacagttggacaacgccactgacctcctgaaggttctccgcaacgg
cggtgctcatctggacttccggacccgagatgggctgacagccgtccactgtgctacccgccagcggaacgcaggggcattgacga
ccctgctggacctgggggcttcgcctgactacaaggacagccgcggcctgacgcccctgtaccatagtgccctagggggggggat
gccctctgttgcgagctgcttctccatgatcatgcacagctggggaccactgatgagaatggttggcaagagatccatcaggcctgtcg
ctttggacacgtgcagcacctggagcaccttttgttctatggggccaacatgggtgctcagaatgcctcgggaaacacagccctgcac
atctgtgccctctacaaccaggagagttgcgcgcgcgtcctgcttttccgtggtgccaacaaggacgtccgcaattacaacagccaga
cagccttccaggtggccattattgcagggaactttgagcttgccgaggtaatcaagacccacaaagactccgatgtcgtaccattcagg
gaaacccccagctatgcaaagcgacggcgtctggctggcccgagtggcctggcatccccacggcccttacagcgctcagccagtga
tatcaacctgaaaggtgatcagcccgcagcttctccagggcccactctccgaagcctccctcatcaactcttgctccagaggcttcagg
aggagaaagaccgtgacagggatggtgaactggagaatgacatcagcggcccctcagcaggcaggggggccacaacaagatca
gccccagtgggcccggcggatccggccccgcgcccggccccggcccggcgtctcccgcgccccccgcgccgccgccccgggg
cccgaagcggaaactttacagtgccgtccccggccgcaagttcatcgctgtgaaggcgcacagcccgcagggcgagggcgagatc
ccgctgcaccgcggcgaggccgtgaaggtgctcagcattggggagggcggtttctgggagggaaccgtgaagggccgaacaggc
tggttcccagctgactgtgtggaagaagtgcagatgcgacagtatgacacccggcatgaaaccagagaggaccggacgaagcgtct
cttccgccactacactgtgggttcctatgacagcctcacttcacacagcgattatgtcatcgatgataaggtggctatcctgcagaaaag
ggaccatgaggggtttggctttgttctccggggagccaaagcagagacccccattgaggagtttacacccacacctgccttccctgca
ctccaataccttgagtctgtagatgtggaaggtgtggcctggagggctggacttcgaactggggacttcctcattgaggtgaacggagt
gaatgtcgtgaaggttggacacaagcaagtggtgggtctcatccgtcagggtggcaaccgcctggtcatgaaggttgtgtctgtgacc
aggaaacccgaggaggatggtgctcggcgcagagccccaccacccccaaagagggctcccagcaccacgctgaccctgcggtcc
aagtccatgacggctgagctcgaggaacttgcttccattcggagaagaaaaggggagaagttggatgagatcctggcagttgccgcg
gagccgacactgaggccggacattgcagatgctgactcgagggcggccactgtcaagcagcggcccaccagccggaggatcacc
cctgctgagatcagctcattgtttgagcgccagggcctcccaggcccagagaagctgccgggctctctgcggaaggggattccacgg
accaaatctgtaggggaggatgagaagctggcatccctactggaagggcgcttcccacgcagcacgtcaatgcaggacacagtgcg
tgaaggtcgaggcattccacccccgccgcagaccgccccgccacccccacccgcgccctactacttcgactccgggccacccccc
accttctcaccgccgccaccaccgggccgggcctatgacactgtgcgctccagcttcaaaccaggcctggaggctcgtctgggtgcg
ggggccgccggcctgtatgatccgagcacgcctctgggcccgctgccctaccctgagcgtcagaagcgtgcgcgctccatgatcat
actgcaggactctgcgccagaagtgggtgatgtcccccggcctgcgcctgccgccacaccgcctgagcgccccaagcgccggcct
cggccgtcaggccctgatagtccctatgccaacctgggcgccttcagtgccagcctcttcgctccgtcgaaaccacagcgccgcaag
agcccgctggtgaagcagcttcaggtggaggacgctcaggagcgcgcggccctggccgtgggtagcccgggaccagtgggcgg
aagctttgcacgagaaccctctccgacgcaccgcgggccccggccaggcagccttgactacagctctggagaaggcctaggcctta
ccttcggcggccctagccctggcccagtcaaggagcggcgcctggaggagcgacgccgttccactgtgttcctctctgtgggtgcca
tcgagggcagccctcccagcgcggatctgccatccctacaaccctcccgctccattgatgagcgcctcctggggacaggcgccacc
actggccgcgatttgctactcccctcccctgtctctgctctgaagccattggtcggtggtcccagccttgggccctcaggctccaccttca
tccaccctctcactggcaaacccttggatcctagctcacccttagctcttgctctggctgcccgggagcgggctctggcctcgcaaaca
ccttcccggtcccccacacctgtgcacagccccgatgctgaccgccctggacccctctttgtggatgtgcaaacccgagactctgaga
gaggaccgttggcttccccagccttctcccctcggagtccagcgtggattccagtgcctgctcggagagaggcagagaagccccctc
gggaagagcggaagtcaccagaggacaagaagtccatgatcctcagcgtcttggacacgtccttgcaacggccagctggcctcattg
ttgtgcatgccaccagcaatgggcaggagcccagcaggctgggggctgaagaggagcgccccggtactccggagctggccccag
cccccatgcaggcagcagctgtggcagagcccatgccaagcccccgggcccagccccctggcagcatcccagcagatcccgggc
caggtcaaggcagctcagaggaggagccagagctggtattcgctgtgaacctgccacctgctcagctgtcctccagcgatgaggag
accagagaggagctggcccgcatagggctagtgccaccccctgaagagtttgccaatgggatcctgctgaccaccccgcccccagg
gccgggccccttgcccaccacggtacccagcccggcctcagggaagcccagcagcgagctgccccctgcccctgagtctgcagct
gactctggagtagaggaggctgacactcgaagctccagtgacccccacctggagaccacaagcaccatttccacagtgtccagcatg
tccaccctgagctcggagagtggggaactcacggacacccacacctcctttgccgatggacacacttttctactcgagaagccaccag
tgcctcccaagcccaagctcaagtccccgctggggaaggggccggtgaccttcagggacccgctgctgaagcaatcctcggacagt
gagctcatggcccagcagcaccatgctgcctctactgggttggcttctgctgctgggcccgcccgccctcgctacctcttccagagaa

ggtccaagctgtggggggaccccgtggagagtcgggggctccctgggcctgaagatgacaaaccaactgtgatcagtgagctcag
ctcccgtctgcagcagctgaataaagacacacgctccttgggggaggaaccagttggtggcctgggcagcctgctggaccctgctaa
gaagtcacccattgcagcagctcggctcttcagcagcctcggtgagctgagcaccatctcagcgcagcgcagcccggggggcccg
ggcggaggggcctcctactcggtgcggcccagcggccggtaccccgtggcgagacgagccccgagcccagtgaaacccgcatc
gctggagcgggtggaggggctggggggggcgtgggaggcgcggggggcccttcggcctcacgcctcccaccatcctcaagtc
gtccagcctctccatcccgcacgaacccaaggaagtgcgcttcgtggtgcgaagtgtgagtgcgcgcagccgctccccctcaccatc
tccgctgccctcgccttctcccggctctggccccagtgccggcccgcgtcggccatttcaacagaagcccctgcagctctggagcaa
gttcgatgtgggcgactggctggagagcatccacttaggcgagcaccgagaccgcttcgaggaccatgagatcgaaggcgcacac
ctgcctgcgctcaccaaggaagacttcgtggagctgggcgtcacacgcgttggccaccgcatgaacatcgagcgtgcgctcaggca
gctggatggcagctga

The Shank3 full length human protein sequence corresponding to GenBank Accession No. Q9BYB0.3 is provided by SEQ ID NO: 6:

MDGPGASAVVVRVGIPDLQQTKCLRLDPAAPVWAAKQRVLCALNHSLQDALNYGL
FQPPSRGRAGKFLDEERLLQEYPPNLDTPLPYLEFRYKRRVYAQNLIDDKQFAKLHT
KANLKKFMDYVQLHSTDKVARLLDKGLDPNFHDPDSGECPLSLAAQLDNATDLLK
VLKNGGAHLDFRTRDGLTAVHCATRQRNAAALTTLLDLGASPDYKDSRGLTPLYHS
ALGGGDALCCELLLHDHAQLGITDENGWQEIHQACRFGHVQHLEHLLFYGADMGA
QNASGNTALHICALYNQESCARVLLFRGANRDVRNYNSQTAFQVAIIAGNFELAEVI
KTHKDSDVVPFRETPSYAKRRRLAGPSGLASPRPLQRSASDINLKGEAQPAASPGPSL
RSLPHQLLLQRLQEEKDRDRDADQESNISGPLAGRAGQSKISPSGPGGPGPAPGPGPA
PPAPPAPPPRGPKRKLYSAVPGRKFIAVKAHSPQGEGEIPLHRGEAVKVLSIGEGGFW
EGTVKGRTGWFPADCVEEVQMRQHDTRPETREDRTKRLFRHYTVGSYDSLTSHSDY
VIDDKVAVLQKRDHEGFGFVLRGAKAETPIEEFTPTPAFPALQYLESVDVEGVAWRA
GLRTGDFLIEVNGVNVVKVGHKQVVALIRQGGNRLVMKVVSVTRKPEEDGARRRA
PPPPKRAPSTTLTLRSKSMTAELEELASIRRRKGEKLDEMLAAAAEPTLRPDIADADS
RAATVKQRPTSRRITPAEISSLFERQGLPGPEKLPGSLRKGIPRTKSVGEDEKLASLLE
GRFPRSTSMQDPVREGRGIPPPPQTAPPPPPAPYYFDSGPPPAFSPPPPPGRAYDTVRSS
FKPGLEARLGAGAAGLYEPGAALGPLPYPERQKRARSMIILQDSAPESGDAPRPPPAA
TPPERPKRRPRPPGPDSPYANLGAFSASLFAPSKPQRRKSPLVKQLQVEDAQERAALA
VGSPGPGGGSFAREPSPTHRGPRPGGLDYGAGDGPGLAFGGPGPAKDRRLEERRRST
VFLSVGAIEGSAPGADLPSLQPSRSIDERLLGTGPTAGRDLLLPSPVSALKPLVSGPSL
GPSGSTFIHPLTGKPLDPSSPLALALAARERALASQAPSRSPTPVHSPDADRPGPLFVD
VQARDPERGSLASPAFSPRSPAWIPVPARREAEKVPREERKSPEDKKSMILSVLDTSL
QRPAGLIVVHATSNGQEPSRLGGAEEERPGTPELAPAPMQSAAVAEPLPSPRAQPPG
GTPADAGPGQGSSEEEPELVFAVNLPPAQLSSSDEETREELARIGLVPPPEEFANGVLL
ATPLAGPGPSPTTVPSPASGKPSSEPPPAPESAADSGVEEADTRSSSDPHLETTSTISTV
SSMSTLSSESGELTDTHTSFADGHTFLLEKPPVPPKPKLKSPLGKGPVTFRDPLLKQSS
DSELMAQQHHAASAGLASAAGPARPRYLFQRRSKLWGDPVESRGLPGPEDDKPTVI
SELSSRLQQLNKDTRSLGEEPVGGLGSLLDPAKKSPIAAARLFSSLGELSSISAQRSPG
GPGGGASYSVRPSGRYPVARRAPSPVKPASLERVEGLGAGAGGAGRPFGLTPPTILK
SSSLSIPHEPKEVRFVVRSVSARSRSPSPSPLPSPASGPGPGAPGPRRPFQQKPLQLWSK
FDVGDWLESIHLGEHRDRFEDHEIEGAHLPALTKDDFVELGVTRVGHRMNIERALRQ
LDGS

In some embodiments, the Shank3 full length human protein sequence corresponding to SEQ ID NO: 6 is encoded by a nucleic acid sequence corresponding to GenBank Accession No. NM_001372044, provided by SEQ ID NO: 16:

atgcagctgagccgcgccgccgccgccgccgccgccgcccctgcggagcccccggagccgctgtcccccgcgccgg
ccccggccccggccccccccggccccctcccgcgcagcgcggccgacggggctccggcgggggggggaagggggggccgggg
cgccgcgcggagtccccgggcgctccgttccccggcgcgagcggccccggcccgggccccggcgcggggatggacggccccg
gggccagcgccgtggtcgtgcgcgtcggcatcccggacctgcagcagacgaagtgcctgcgcctggacccggccgcgcccgtgt
gggccgccaagcagcgcgtgctctgcgccctcaaccacagcctccaggacgcgctcaactatgggcttttccagccgccctcccgg
ggccgcgccggcaagttcctggatgaggagcggctcctgcaggagtacccgcccaacctggacacgcccctgccctacctggagtt
tcgatacaagcggcgagtttatgcccagaacctcatcgatgataagcagtttgcaaagcttcacacaaaggcgaacctgaagaagttc
atggactacgtccagctgcatagcacggacaaggtggcacgcctgttggacaaggggctggaccccaacttccatgaccctgactca
ggagagtgccccctgagcctcgcagcccagctggacaacgccacggacctgctaaaggtgctgaagaatggtggtgcccacctgg
acttccgcactcgcgatgggctcactgccgtgcactgtgccacacgccagcggaatgcggcagcactgacgaccctgctggacctg
ggggcttcacctgactacaaggacagccgcggcttgacacccctctaccacagcgccctggggggtggggatgccctctgctgtga
gctgcttctccacgaccacgctcagctggggatcaccgacgagaatggctggcaggagatccaccaggcctgccgctttgggcacg
tgcagcatctggagcacctgctgttctatggggcagacatgggggcccagaacgcctcggggaacacagccctgcacatctgtgcc
ctctacaaccaggagagctgtgctcgtgtcctgctcttccgtggagctaacagggatgtccgcaactacaacagccagacagccttcc
aggtggccatcatcgcagggaactttgagcttgcagaggttatcaagacccacaaagactcggatgttgtaccattcagggaaacccc
cagctatgcgaagcggcggcgactggctggccccagtggcttggcatcccctcggcctctgcagcgctcagccagcgatatcaacct
gaagggggaggcacagccagcagcttctcctggaccctcgctgagaagcctcccccaccagctgctgctccagcggctgcaagag
gagaaagatcgtgaccgggatgccgaccaggagagcaacatcagtggccctttagcaggcagggccggccaaagcaagatcagc
ccgagcgggcccggcggccccggccccgcgcccggccccggccccgcgccccctgcgccccccgcaccgccgccccggggc
ccgaagcggaaactttacagcgccgtccccggccgcaagttcatcgccgtgaaggcgcacagcccgcagggtgaaggcgagatcc
cgctgcaccgcggcgaggccgtgaaggtgctcagcattggggagggcggtttctgggagggaaccgtgaaaggccgcacgggct
ggttcccggccgactgcgtggaggaagtgcagatgaggcagcatgacacacggcctgaaacgcgggaggaccggacgaagcgg
ctctttcggcactacacagtgggctcctacgacagcctcacctcacacagcgattatgtcattgatgacaaagtggctgtcctgcagaaa
cgggaccacgagggctttggttttgtgctccggggagccaaagcagagacccccatcgaggagttcacgcccacgccagccttccc
ggcgctgcagtatctcgagtcggtggacgtggagggtgtggcctggagggccgggctgcgcacgggagacttcctcatcgaggtg
aacggggtgaacgtggtgaaggtcggacacaagcaggtggtggctctgattcgccagggtggcaaccgcctcgtcatgaaggttgt
gtctgtgacaaggaagccagaagaggacggggctcggcgcagagccccaccgccccccaagagggcccccagcaccacactga
ccctgcgctccaagtccatgacagctgagctcgaggaacttgcctccattcggagaagaaaaggggagaagctggacgagatgctg
gcagccgccgcagagccaacgctgcggccagacatcgcagacgcagactccagagccgccaccgtcaaacagaggcccaccag
tcggaggatcacacccgccgagattagctcattgtttgaacgccagggcctcccaggcccagagaagctgccgggctccttgcggaa
ggggattccacggaccaagtctgtaggggaggacgagaagctggcgtccctgctggaagggcgcttcccgcggagcacctcgatg
caagacccggtgcgcgagggtcgcggcatcccgcccccgccgcagaccgcgccgcctcccccgcccgcgccctactacttcgact
cggggccgcccccggccttctcgccgccgcccccgccgggccgcgcctacgacacggtgcgctccagcttcaagcccggcctgg
aggcgcgcctgggcgcgggcgctgccggcctgtacgagccgggcgcggccctcggcccgctgccgtatcccgagcggcagaag
cgcgcgcgctccatgatcatcctgcaggactcggcgcccgagtcgggcgacgcccctcgacccccgcccgcggccaccccgccc
gagcgacccaagcgccggccgcggccgcccggccccgacagcccctacgccaacctgggcgccttcagcgccagcctcttcgct
ccgtccaagccgcagcgccgcaagagccccctggtgaagcagctgcaggtggaggacgcgcaggagcgcgcggccctggccgt
gggcagccccggtcccggcggcggcagcttcgcccgcgagccctccccgacccaccgcggtccgcgcccgggtggcctcgacta
cggcgcgggcgatggcccggggctcgcgttcggcggcccgggcccggccaaggaccggcggctggaggagcggcgccgctcc
actgtgttcctgtccgtgggggccatcgagggcagcgcccccggcgcggatctgccatccctacagccctcccgctccatcgacgag
cgcctcctggggaccggccccaccgccggccgcgacctgctgctgccctccccggtgtctgccctgaagccgttggtcagcggccc
gagcctggggccctcgggttccaccttcatccacccactcaccggcaaacccctggaccccagctcacccctggcccttgccctggc
tgcccgagagcgagctctggcctcccaggcgccctcccggtcccccacacccgtgcacagtcccgacgccgaccgccccggacc
cctgtttgtggatgtacaggcccgggacccagagcgagggtccctggcttccccggctttctccccacggagcccagcctggattcct
gtgcctgctcgcagggaggcagagaaggtcccccgggaggagcggaagtcacccgaggacaagaagtccatgatcctcagcgtc
ctggacacatccctgcagcggccagctggcctcatcgttgtgcacgccaccagcaacgggcaggagcccagcaggctggggggg
gccgaagaggagcgcccgggcaccccggagttggccccggcccccatgcagtcagcggctgtggcagagcccctgcccagccc
ccgggcccagccccctggtggcaccccggcagacgccgggccaggccagggcagctcagaggaagagccagagctggtgtttg
ctgtgaacctgccacctgcccagctgtcgtccagcgatgaggagaccagggaggagctggcccgaattgggttggtgccaccccct
gaagagtttgccaacggggtcctgctggccaccccactcgctggcccgggcccctcgcccaccacggtgcccagcccggcctcag
ggaagcccagcagtgagccaccccctgcccctgagtctgcagccgactctggggggaggaggctgacacacgcagctccagcg
acccccacctggagaccacaagcaccatctccacggtgtccagcatgtccaccttgagctcggagagcggggaactcactgacacc
cacacctccttcgctgacggacacacttttctactcgagaagccaccagtgcctcccaagcccaagctcaagtccccgctggggaag
gggccggtgaccttcagggacccgctgctgaagcagtcctcggacagcgagctcatggcccagcagcaccacgccgcctctgccg
ggctggcctctgccgccgggcctgcccgccctcgctacctcttccagagaaggtccaagctatggggggaccccgtggagagccg
ggggctccctgggcctgaagacgacaaaccaactgtgatcagtgagctcagctcccgcctgcagcagctgaacaaggacacgcgtt
ccctgggggaggaaccagttggtggcctgggcagcctgctggaccctgccaagaagtcgcccatcgcagcagctcggctcttcagc
agcctcggtgagctgagctccatttcagcgcagcgcagccccgggggcccgggggggggcctcgtactcggtgaggcccagt
ggccgctaccccgtggcgagacgcgccccgagcccggtgaagcccgcgtcgctggagcgggtggaggggctggggggggcg
cggggggcgcagggcggcccttcggcctcacgccccccaccatcctcaagtcgtccagcctctccatcccgcacgagcccaagga
ggtgcgcttcgtggtgcgcagcgtgagcgcgcgcagtcgctccccctcgccgtcgccgctgccctcgcccgcgtccggccccggc
cccggcgcccccggcccacgccgacccttccagcagaagccgctgcagctctggagcaagttcgacgtgggcgactggctggag
agcatccacctaggcgagcaccgcgaccgcttcgaggaccatgagatagaaggcgcgcacctacccgcgcttaccaaggacgact
tcgtggagctgggcgtcacgcgcgtgggccaccgcatgaacatcgagcgcgcgctcaggcagctggacggcagctga

The full-length Shank3 protein comprises multiple domains and is encoded by a gene that is about 5.2 Kb in size. Due to its size, it is difficult to deliver full-length Shank3 to a tissue or cell of interest via an AAV vector. As disclosed in and incorporated by reference from PCT Publication No. WO2022/040239 and US Patent Publication No. US2023/0340041, specific domains can be removed or truncated from the full-length Shank3 protein to produce MiniShank3 that is efficacious in restoring Shank3 activity in excitatory neurons). Shank proteins (e.g., Shank3 proteins) encoded by polynucleotides described herein can be miniaturized to form a shortened variant of the native, full length Shank3 protein. As disclosed herein, a miniaturized Shank3 protein, or a DNA construct encoding the miniaturized Shank3 protein, are referred to interchangeably as “miniShank3” or “MiniShank3.”

In some embodiments, the Shank3 protein disclosed herein is expressed as a miniaturized Shank3 DNA construct. In some embodiments, the variant Shank3 DNA constructs and the Shank3 proteins disclosed herein (MiniShank3) comprise fewer domains than the full-length Shank3 gene and protein. In some embodiments, the Shank3 protein disclosed herein is encoded by a non-naturally occurring polynucleotide.

Shank3 proteins encoded by polynucleotides described herein can include one or more protein domains. For example, Shank3 proteins can include one or more of: an SH3 domain, a PDZ domain, a Homer binding domain, a Cortactin domain, a SAM domain, and/or an ankyrin repeat domain.

In some embodiments, the SH3 domain comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or is 100% identical, including all values in between, to residues 474-525 of SEQ ID NO: 6 or residues 473-524 of SEQ ID NO: 5. In some embodiments, the SH3 domain comprises at least 90% identity to residues 474-525 of SEQ ID NO: 6. In some embodiments, the SH3 domain comprises at least 90% identity to residues 473-524 of SEQ ID NO: 5. In some embodiments, the SH3 domain comprises residues 474-525 of SEQ ID NO: 6. In some embodiments, the SH3 domain comprises residues 473-524 of SEQ ID NO: 5. In some embodiments, the SH3 domain can comprise any percent identity to residues 474-525 of SEQ ID NO: 6 suitable for construction of the MiniShank3. In some embodiments, the SH3 domain can comprise any percent identity to residues 473-524 of SEQ ID NO: 5 suitable for construction of the MiniShank3.

In some embodiments, the PDZ domain comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or is 100% identical, including all values in between, to residues 573-662 of SEQ ID NO: 6 or residues 572-661 of SEQ ID NO: 5. In some embodiments, the PDZ domain comprises at least 90% identity to residues 573-662 of SEQ ID NO: 6. In some embodiments, the PDZ domain comprises at least 90% identity to residues 572-661 of SEQ ID NO: 5. In some embodiments, the PDZ domain comprises residues 573-662 of SEQ ID NO: 6. In some embodiments, the PDZ domain comprises residues 572-661 of SEQ ID NO: 5. In some embodiments, the PDZ domain can comprise any percent identity to residues 573-662 of SEQ ID NO: 6 suitable for construction of the MiniShank3. In some embodiments, the PDZ domain can comprise any percent identity to residues 572-661 of SEQ ID NO: 5 suitable for construction of the MiniShank3.

In some embodiments, the Homer binding domain comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or is 100% identical, including all values in between, to residues 1294-1323 of SEQ ID NO: 5 or 6. In some embodiments, the Homer domain comprises at least 90% identity to residues 1294-1323 of SEQ ID NO: 5. In some embodiments, the Homer domain comprises at least 90% identity to residues 1294-1323 of SEQ ID NO: 6. In some embodiments, the Homer domain comprises residues 1294-1323 of SEQ ID NO: 5 or 6. In some embodiments, the Homer domain can comprise any percent identity to residues 1294-1323 of SEQ ID NO: 5 or 6 suitable for construction of the MiniShank3.

In some embodiments, the Cortactin binding domain comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or is 100% identical, including all values in between, to residues 1400-1426 of SEQ ID NO: 5 or 6. In some embodiments, the Cortactin binding domain comprises at least 90% identity to residues 1400-1426 of SEQ ID NO: 5. In some embodiments, the Cortactin binding domain comprises at least 90% identity to residues 1400-1426 of SEQ ID NO: 6. In some embodiments, the Cortactin binding domain comprises residues 1400-1426 of SEQ ID NO: 5 or 6. In some embodiments, the Cortactin binding domain can comprise any percent identity to residues 1400-1426 of SEQ ID NOs: 5 or 6 suitable for construction of the MiniShank3.

In some embodiments, the SAM domain comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or is 100% identical, including all values in between, to residues 1664-1729 of SEQ ID NO: 6 or to residues 1663-1728 of SEQ ID NO:5. In some embodiments, the SAM binding domain comprises at least 90% identity to residues 1664-1729 of SEQ ID NO: 6. In some embodiments, the SAM binding domain comprises at least 90% identity to residues 1663-1728 of SEQ ID NO: 5. In some embodiments, the SAM domain comprises residues 1664-1729 of SEQ ID NO: 6. In some embodiments, the SAM domain comprises residues 1663-1728 of SEQ ID NO:5. In some embodiments, the SAM binding domain can comprise any percent identity to residues 1664-1729 of SEQ ID NO: 6 suitable for construction of the MiniShank3. In some embodiments, the SAM binding domain can comprise any percent identity to residues 1663-1728 of SEQ ID NO: 5 suitable for construction of the MiniShank3.

In some embodiments, the ankyrin repeat domain comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or is 100% identical, including all values in between, to residues 148-345 of SEQ ID NO: 6 or to residues 147-313 of SEQ ID NO: 5. In some embodiments, the ankyrin repeat domain comprises at least 90% identity to residues 148-345 of SEQ ID NO: 6. In some embodiments, the ankyrin repeat domain comprises at least 90% identity to residues 147-313 of SEQ ID NO: 5. In some embodiments, the ankyrin repeat domain can comprise any percent identity to residues 148-345 of SEQ ID NO: 6 suitable for construction of the MiniShank3. In some embodiments, the ankyrin repeat domain can comprise any percent identity to residues 147-313 of SEQ ID NO: 5 suitable for construction of the MiniShank3.

In some embodiments, the MiniShank3 protein is less than 65% identical to SEQ ID NO: 5 over the full length of SEQ ID NO: 5. In some embodiments, the MiniShank3 protein is less than 65% identical to SEQ ID NO: 6 over the full length of SEQ ID NO: 6. As used herein, “less than 65%” includes any percent identity less than 65% that is suitable for construction of the MiniShank3. In some embodiments, the MiniShank3 protein is less than 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11% or 10% identical to SEQ ID NO: 5 over the full length of SEQ ID NO: 5.

In some embodiments, the MiniShank3 protein comprises an amino acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical, or is 100% identical, including all values in between, to any one of SEQ ID NOs: 17-20, provided in Table 1.

In some embodiments, the MiniShank3 protein comprises any one of SEQ ID NOs: 17-20. In some embodiments, SEQ ID NO: 17 is encoded by SEQ ID NO: 1. In some embodiments, SEQ ID NO: 18 is encoded by SEQ ID NO: 2. In some embodiments, SEQ ID NO: 19 is encoded by SEQ ID NO: 3. In some embodiments, SEQ ID NO: 20 is encoded by SEQ ID NO: 4.

In some embodiments, the MiniShank3 protein comprises an ankyrin repeat domain. In certain embodiments in which the MiniShank3 protein comprises an ankyrin repeat domain, the MiniShank3 protein comprises SEQ ID NOs: 19 and/or 20.

In other embodiments, the MiniShank3 protein does not comprise an ankyrin repeat domain. In certain embodiments in which the MiniShank3 protein does not comprise an ankyrin repeat domain, the MiniShank3 protein comprises SEQ ID NOs: 17 and/or 18.

In some embodiments, the sequences of polynucleotides encoding MiniShank3 proteins associated with the disclosure comprise at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or are 100% identical, including all values in between, to any one of SEQ ID NOs: 1-4, and encode one or more proteins with Shank3 activity. In some embodiments, the sequences of polynucleotides encoding MiniShank3 proteins associated with the disclosure comprise at least 90% identity to any one of SEQ ID NOs: 1-4, and encode one or more proteins with Shank3 activity. In some embodiments, the sequences of polynucleotides encoding MiniShank3 proteins associated with the disclosure comprise any one of SEQ ID NOs: 1-4. In some embodiments, any one of SEQ ID NOs: 1-4 encodes one or more proteins with partial Shank3 activity. In some embodiments, any one of SEQ ID NOs: 1-4 encodes one or more proteins with full Shank3 activity.

In some embodiments, the MiniShank3 is encoded by any one of SEQ ID NOs: 1-4, provided in Table 1. SEQ ID NO: 1 and SEQ ID NO: 3 correspond to mouse MiniShank3 nucleic acid sequences, while SEQ ID NO: 2 and SEQ ID NO: 4 correspond to human MiniShank3 nucleic acid sequences. SEQ ID NO: 1 and SEQ ID NO: 2 encode MiniShank3 proteins that do not comprise an ankyrin repeat domain or the N-terminal domain. SEQ ID NO: 3 and SEQ ID NO: 4 encode MiniShank3 proteins that comprise an ankyrin repeat domain and the N-terminal domain.

Polynucleotides described herein that encode MiniShank3 proteins encode proteins that have at least partial Shank3 activity.

As disclosed herein, “identity” of sequences refers to the measurement or calculation of the percent of identical matches between two or more sequences with gap alignments addressed by a mathematical model, algorithm, or computer program that is known to one of ordinary skill in the art. The percent identity of two sequences (e.g., nucleic acid or amino acid sequences) may, for example, be determined using Basic Local Alignment Search Tool (BLAST®) such as NBLAST® and XBLAST® programs (version 2.0). Alignment technique such as Clustal Omega may be used for multiple sequence alignments. Other algorithms or alignment methods may include but are not limited to the Smith-Waterman algorithm, the Needleman-Wunsch algorithm, or Fast Optimal Global Sequence Alignment Algorithm (FOGSAA).

In some embodiments, a polynucleotide encoding the Shank protein as disclosed herein (Shank 1, Shank2, Shank3) is less than about 4.6 kb, about 4.5 kb, about 4.4 kb, about 4.3 kb, about 4.2 kb, about 4.1 kb, about 4.0 kb, about 3.9 kb, about 3.8 kb, about 3.7 kb, about 3.6 kb, about 3.5 kb, about 3.4 kb, about 3.3 kb, about 3.2 kb, about 3.1 kb, about 3.0 kb, about 2.9 kb, about 2.8 kb, about 2.7 kb, about 2.6 kb, about 2.5 kb, about 2.4 kb, about 2.3 kb, about 2.2 kb, or about 2.1 kb in size. In some embodiments, the polynucleotide encoding the Shank protein as disclosed herein (Shank 1, Shank2, Shank3) can be in any size that is suitable for the methods and vectors disclosed in the present disclosure.

### Methods of Treatment

### Diseases and Disorders

The present disclosure provides compositions and methods suitable for treating a neurodevelopmental disorder, such as an autism spectrum disorder (ASD), or Phelan-McDermid syndrome. Provided are methods of treating or ameliorating symptoms of an autism spectrum disorder (ASD) or Phelan-McDermid syndrome or a syndrome associated with a SHANK3 mutation in a primate subject in need thereof, the method comprising administering to the primate subject a therapeutically effective amount of a composition comprising an AAV vector, wherein the AAV vector comprises a) an artificial genome comprising a polynucleotide encoding a transgene comprising a miniShank3 protein comprising an amino acid sequence at least 90% identical to any one of SEQ ID NOs: 17-20 and has SHANK3 activity, wherein the transgene is operably linked to an hSYN promoter comprising SEQ ID NO: 22 and b) a capsid protein, wherein the capsid protein is AAV9 or a variant thereof.

As used herein “neurodevelopmental disorder” refers to any disorder that impairs the growth and/or development of the brain and/or central nervous system. In some embodiments, neurodevelopmental disorders impact one or more brain functions, such as emotion, learning ability, self-control, and memory. It should be appreciated that aspects of the disclosure may be applicable for treatment of any neurodevelopmental disorder.

In some embodiments, the neurodevelopmental disorder is an autism spectrum disorder (ASD). Diagnosis of ASDs is mainly based on criteria such as deficits in communication, impaired social interaction, and repetitive or restricted interests and behaviors. ASDs are highly heritable disorders with concordance rates as high as 90% for monozygotic twins. However, ASDs are clinically heterogeneous, covering a wide range of discrete disorders of differential symptomatic severity. ASDs are believed to be etiologically heterogeneous, possibly encompassing polygenic, monogenic and environmental factors.

Alterations in synaptic connectivity and function have been proposed as a key mechanism underlying ASDs. Recent genetic studies have identified a large numbers of candidate genes for ASDs, many of which encode synaptic proteins including Shank3, Neuroligin-3, Neuroligin-4 and Neurexin-1. These findings suggest that synaptic dysfunction may underlie a common mechanism for a subset of ASDs. Various Shank3 mutations have been identified as a monogenic cause of ASD with intellectual disability (ID). In ASD patients, all Shank3 deletions and/or mutations that have been identified lead to loss of function (LoF) in one of the two normal copies of the Shank3 gene (i.e. haploinsufficiency). Recent genetic screens also identified a large number of mutations in the Shank3 gene including microdeletions, nonsense mutations and recurrent breakpoints in ASD patients not diagnosed with Phelan-McDermid syndrome (PMS). These implicate Shank3 gene disruption and/or mutation as a monogenic cause of autism spectrum disorder (ASD). The current estimation is that deletions and/or mutations involving Shank3 account for about 2% of all ASD patients with ID. Thus, understanding the function of Shank3 may provide insight into pathological mechanisms of ASD.

As used herein, “intellectual disability” refers to a disability that causes a subject to have deficits in intellectual functioning and/or adaptive functioning. Intellectual functioning can include, for example, reasoning, problem solving, planning, abstract thinking, judgment, academic learning, and/or experiential learning. Intellectual functioning can be measured using any method known in the art, such as by IQ tests. Adaptive functioning can include, for example, skills needed to live in an independent and responsible manner such as communication and social skills. In some instances, intellectual disability can be evident during childhood or adolescence.

In some embodiments, the neurodevelopmental disorder is Phelan-McDermid syndrome (PMS, 22q13.3 deletion syndrome), which is an autism spectrum disorder that shows autistic-like behaviors, hypotonia, severe intellectual disability and impaired development of speech and language. Shank3 is one of the genes that has been reported to be deleted in Phelan-McDermid syndrome. Disruption of Shank3 is thought to be the cause of the core neurodevelopmental and neurobehavioral deficits in Phelan-McDermid syndrome because individuals carrying a ring chromosome 22 with an intact Shank3 gene are phenotypically normal.

Other neurodevelopmental disorders can include but are not limited to attention-deficit/hyperactivity disorder (ADHD), learning disabilities such as dyslexia or dyscalculia, intellectual disability (mental retardation), conduct or motor disorders, cerebral palsy, impairments in vision and hearing, developmental language disorder, neurogenetic disorders such as Fragile X syndrome, Down syndrome, Rett syndrome, hypogonadotropic hypogonadal syndromes, and traumatic brain injury.

In embodiments, the subject in need of treatment has poor response to negative stimuli, such as noises and scary masks. In embodiments, the primate subject has at about 3 months, about 6 months or about one year after said administering a decreased response to negative stimuli compared to the primate subject before treatment or a similarly situated untreated control primate. In embodiments, the decreased response to negative stimuli is statistically significant and/or clinically significant improvement relative to the pre-treatment subject or based upon natural history of the disease.

In embodiments, the subject in need of treatment has low motivation, flexibility and/or memory. In embodiments, the primate subject has at about 3 months, about 6 months or about one year after said administering increased motivation, flexibility, and memory compared to the primate subject before treatment or a similarly situated untreated control primate. In embodiments, the increased motivation, flexibility, and memory is statistically significant and/or clinically significant improvement relative to the pre-treatment subject or based upon natural history of the disease.

In embodiments, the subject in need of treatment has a low response to reward stimuli. In embodiments, the primate subject at about 3 months, about 6 months or about one year after said administering has an increased response to reward compared to the primate subject before treatment or a similarly situated untreated control primate. In embodiments, the increased response to a reward is statistically significant and/or clinically significant improvement relative to the pre-treatment subject or based upon natural history of the disease.

In embodiments, the subject in need of treatment has a cognitive deficit and/or intellectual disability. In embodiments, the primate subject at about 3 months, about 6 months or about one year after said administering has decreased cognitive deficit and/or intellectual disability compared to the primate subject before treatment or a similarly situated untreated control primate. In embodiments, the decreased cognitive deficit and/or intellectual disability is statistically significant and/or clinically significant improvement relative to the pre-treatment subject or based upon natural history of the disease.

In embodiments, the subject in need of treatment has abnormal fMRI connectivity in various parts of the brain. In embodiments, the primate subject at about 3 months, about 6 months or about one year after said administering has increased resting fMRI connectivity lower in anterior/medial areas compared to the primate subject before treatment or a similarly situated untreated control primate. In embodiments, the primate subject at about 3 months, about 6 months or about one year after said administering has decreased resting fMRI connectivity lower in posterior/lateral (sensory) areas compared to the primate subject before treatment or a similarly situated untreated control primate. In embodiments, the primate subject after said administering has MRI improvements more than 12 months or more than 18 months or more than two years after administration of the therapeutically effective amount of the composition. In embodiments, the improved fMRI connectivity is statistically significant and/or clinically significant improvement relative to the pre-treatment subject or based upon natural history of the disease.

### Subjects

A subject to be treated by methods described herein may be a human subject or a non-human subject. Non-human subjects include, for example: non-human primates; farm animals, such as cows, horses, goats, sheep, and pigs; pets, such as dogs and cats; and rodents. In embodiments, the primate subject is human.

A subject to be treated by methods described herein may be a subject having, suspected of having, or at risk for developing a neurodevelopmental disorder. In some embodiments, a subject has been diagnosed as having a neurodevelopmental disorder, while in other embodiments, a subject has not been diagnosed as having a neurodevelopmental disorder. In some embodiments, the subject is a human subject having, suspected of having, or at risk for developing an autism spectrum disorder (ASD). In some embodiments, the subject is a human subject having, suspected of having, or at risk for developing Phelan-McDermid syndrome. In some embodiments, the subject is a subject having a Shank3 gene mutation. In some embodiments, the subject is a subject having reduced expression of the Shank3 gene relative to a control subject. In some embodiments, the expression of the Shank3 gene is reduced in the subject by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control subject. In some embodiments, the control subject is a subject that does not have, is not suspected of having, or is not at risk of having, a neurodevelopmental disorder. In some embodiments, the reduced expression of the Shank3 gene in a subject is caused by disruption of at least one copy of the Shank3 gene. In some embodiments, the disruption of the Shank3 gene comprises a deletion in at least one copy of the Shank3 gene. In some embodiments, the disruption of the Shank3 gene comprises one or more mutations within at least one copy of the Shank3 gene.

In some embodiments, the subject is a human subject who exhibits one or more symptoms of an ASD. In some embodiments, the subject is a human subject who exhibits developmental delay. In some embodiments, the subject is a human subject who exhibits intellectual disability (ID). In some embodiments, the subject is a human subject who exhibits sleep disturbance. In some embodiments, the subject is a human subject who exhibits hypotonia. In some embodiments, the subject is a human subject who exhibits lack of speech. In some embodiments, the subject is a human subject who exhibits language delay. In some embodiments, the subject is a human subject who exhibits any symptoms or signs of an ASD.

In some embodiments, a subject is a human subject who is an adult. In some embodiments, the adult is older than 25 years of age. In some embodiments, the adult is not older than 25 years of age. In some embodiments, the adult is not older than 21 years of age. In some embodiments, the adult is not older than 18 years of age. In some embodiments, the adult is 16 years of age. In some embodiments, a subject is elderly (e.g., 65 years old or older). In some embodiments, the adult can be any age of adulthood that is suitable for the treatment disclosed herein.

In some embodiments, the subject is a human subject who is not an adult. In some embodiments, the human subject is not older than 16 years of age. In some embodiments, the human subject is not older than 10 years of age. In some embodiments, the human subject is 10 years of age or younger. In some embodiments, the human subject is 3 years of age or younger. In some embodiments, the human subject is a child or an infant. In some embodiments, the human subject is a toddler. In some embodiments, the human subject is at the fetal stage of development. In some embodiments, the human subject is at the prenatal stage of development.

### Viral Vectors

As disclosed herein, polynucleotides encoding MiniShank3 proteins can be delivered to a tissue or cell of interest in a viral vector. Vectors described herein can be used to deliver a nucleic acid encoding a protein of interest to a subject, including, e.g., delivery to specific organs or to the central nervous system (CNS) of a subject. In some embodiments, the protein of interest is a Shank protein. In some embodiments, the protein of interest is a Shank3 protein. In some embodiments, the protein of interest is a MiniShank3 protein.

In some embodiments, the present disclosure provides a vector comprising a polynucleotide encoding a Shank protein disclosed herein. In some embodiments, the present disclosure provides a vector comprising a polynucleotide encoding a Shank3 protein. In some embodiments, the vector is a viral vector. In some embodiments, the vector is an AAV vector.

AAV refers to a replication-deficient Dependoparvovirus within the Parvoviridae genus of viruses. AAV can be derived from a naturally occurring virus or can be recombinant. AAV can be packaged into capsids, which can be derived from naturally occurring capsid proteins or recombinant capsid proteins. The single-stranded DNA genome of AAV includes inverted terminal repeat (ITRs). ITRs are involved in the replication and encapsidation of the AAV genome, along with its integration in the host genome and its excision. Without wishing to be bound by any theory, AAV vectors can comprise one or more ITRs, including a 5′ ITR and/or a 3′ ITR, one or more promoters, one or more nucleic acid sequences encoding one or more proteins of interest, and/or additional posttranscriptional regulator elements. AAV vectors disclosed herein can be prepared using standard molecular biology techniques known to one of ordinary skill in the art, as described, for example, in Sambrook et. al. (Molecular Cloning: A Laboratory Manual. Cold Spring Harbor Laboratory Press, N.Y. (2012)), which is incorporated herein by reference in its entirety.

In some embodiments, AAV integrates into a host cell genome. In some embodiments, AAV does not integrate into a host genome. In some embodiments, AAV vectors disclosed herein can include sequences from any known organism. In some embodiments, AAV vectors disclosed herein can include synthetic sequences. AAV vector sequences can be modified in any way known to one of ordinary skill in the art, such as by incorporating insertions, deletions or substitutions, and/or through the use of posttranscriptional regulatory elements, such as promoters, enhancers, and transcription and translation terminators, such as polyadenylation signals. In some embodiments, AAV vectors can include sequences related to replication and integration.

In some embodiments, a MiniShank3 as disclosed herein is delivered to a tissue or a cell of interest via an AAV vector. In some embodiments, the AAV vector delivering the MiniShank3 as disclosed herein is delivered to the central nervous system (CNS) of a subject. As used herein, delivering the AAV vector to the CNS may include delivering the AAV vector to any tissue or cell of interest in the CNS. In some embodiments, delivering the AAV vector to the CNS involves delivering the AAV vector to neuronal tissues or cells. In some embodiments, delivering the AAV vector to the CNS involves delivering the AAV vector to the brain. In some embodiments, delivering the AAV vector to the CNS involves delivering the AAV vector to the spinal cord. In some embodiments, delivering the AAV vector to the CNS involves delivering the AAV vector to the white and gray matter. In some embodiments, the AAV vector delivering the MiniShank3 as disclosed herein is delivered to any tissue or cell of interest of a subject that is suitable for the treatments as disclosed herein.

As used in the present disclosure, “delivering” or “administering” an AAV vector can include any method known in the art for delivering or administering an AAV vector or a composition comprising an AAV vector to a subject. Administering can include but is not limited to direct administration of an AAV vector or a composition comprising the AAV vector, or peripheral administration via passive diffusion or convection-enhanced delivery (CED) to bypass the blood brain barrier as known in the art. AAV vectors described herein can be administered in any composition that would be compatible with aspects of the disclosure.

AAV vectors can include any known AAV serotype, including, for example, AAV1, AAV2, AAV3, AAV4, AAV5, AAV6, AAV7, AAV8, AAV9, AAV10, and AAV11. In some embodiments, the AAV serotype is AAV9. Clades of AAV viruses are described in, and incorporated by reference, from Gao et al. (2004) J. Virol. 78(12):6381-6388. In some embodiments, any AAV serotype that is suitable for delivery to the CNS may be selected.

AAV vectors of the present disclosure may comprise or be derived from any natural or recombinant AAV serotype. In some embodiments, the AAV vector may utilize or be based on an AAV serotype described in WO 2017/201258A1, the contents of which are incorporated herein by reference in its entirety, such as, but not limited to, AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK103, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF1l/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, AAV-PHP.B (PHP.B), AAV-PHP.A (PHP.A), G2B-26, G2B-13, TH1.1-32 and/or TH1.1-35, and variants thereof. AAV vectors are described further in U.S. Pat. No. 9,585,971, US 2017/0166926, and WO2020/160337, which are incorporated by reference herein in their entireties.

In some embodiments, a MiniShank3 disclosed herein is delivered by an AAV vector. In some embodiments, the AAV vector comprises a transgene and its regulatory sequences, and optionally 5′ and 3′ ITRs. In some embodiments, the transgene and its regulatory sequences are flanked by the 5′ and 3′ ITR sequences. The transgene may comprise, as disclosed herein, one or more regions that encode a MiniShank3. The transgene may also comprise a region encoding for another protein. The transgene may also comprise one or more expression control sequences (e.g., a poly-A tail). In some embodiments, an AAV vector comprises at least AAV ITRs and a MiniShank3 transgene.

In some embodiments, the AAV may be packaged into an AAV particle and administered to a subject and/or delivered to a selected target cell. In some embodiments, the AAV particle comprises an AAV capsid protein. In some embodiments, the AAV particle comprises at least one capsid protein that is selected from the AAV serotypes as disclosed herein including AAV1, AAV2, AAV3, AAV4, AAV5, AAV6, AAV6.2, AAV7, AAV8, AAV9, PHB.eB, AAV.rh8, AAV.rh10, AAV.rh39, AAV.43, AAV2/2-66, AAV2/2-84, and AAV2/2-125, or a variant of any of the foregoing.

In some embodiments, the miniShank3 transgene coding sequence in the AAV vector is operably linked to regulatory sequences for tissue-specific gene expression. In some cases, the tissue-specific regulatory sequences bind tissue-specific transcription factors that induce transcription in a tissue specific manner. Such tissue-specific regulatory sequences (e.g., promoters, enhancers, etc.) are well known in the art. In some embodiments, the tissue-specific regulatory sequence can be a Syn promoter (e.g., hSyn1). In some embodiments, the tissue-specific regulatory sequence can be any promoter or enhancer that is neuron-specific and is suitable for the treatments described herein.

In some embodiments, a miniShank3 transgene coding sequence comprising SEQ ID NO: 2 or 4 in an AAV vector is operably linked to a promoter and is flanked by AAV ITRs. In some embodiments, a miniShank3 transgene coding sequence comprising SEQ ID NO: 1 or 3 in an AAV vector is operably linked to a promoter and is flanked by AAV ITRs.

Aspects of the disclosure relate to AAV vectors expressing miniShank3 transgenes. In some embodiments, a miniShank3 transgene is flanked by AAV ITRs. In some embodiments, the AAV ITRs comprise AAV2 ITRs. In some embodiments, the AAV ITRs comprise AAV1 ITRs. In some embodiments, the AAV ITRs comprise AAV5 ITRs. In some embodiments, the AAV ITRs comprise AAV6 ITRs. In some embodiments, the AAV ITRs comprise AAV8 ITRs. In some embodiments, the AAV ITRs comprise AAV9 ITRs. In some embodiments, the AAV ITRs comprise rh10 ITRs. In some embodiments, the AAV ITRs may include self-complementary ITRs.

It should be appreciated that AAV vectors described herein can include DNA constructs that comprise a transgene such as MiniShank3, 5′ and/or 3′ ITRs, promoters, introns, and/or other associated regulatory elements that are known in the art.

In some embodiments, the AAV vector comprises a Woodchuck Hepatitis Virus Posttranscriptional Regulatory Element (WPRE), which may enhance miniShank3 transgene expression. In some embodiments, the AAV vector comprises an untranslated portion such as an intron or a 5′ or 3′ untranslated region. In some embodiments, the intron may be located between the promoter/enhancer sequence and the miniShank3 transgene.

In some embodiments, the AAV vector used herein may be a self-complementary vector.

SEQ ID NO: 21 comprises a human Mini-Shank3 gene, a 5′-ITR, a 3′-ITR, a WPRE, an hGH polyA, an F1 origin, a NeoR/KanR marker, a hSyn1 promoter, and a PUC origin. In some embodiments, the Inverted terminal repeat (ITR) sequences comprise about 145 nucleotides each. These elements may be useful in cis for effective replication and encapsidation. A skilled person in the art would appreciate that any elements of AAV vectors known in the art may be compatible with aspects of the disclosure. One of skill in the art would also appreciate that any of the polynucleotide sequences described herein that encode a functional MiniShank3 protein can be expressed in a DNA construct for AAV delivery. These DNA constructs may include one or more elements, such as: a 5′ ITR, a 3′ ITR, a WPRE such as WPRE3, a polyA such as an hGH polyA (including modified versions of an hGH polyA), an F1 origin, a NeoR/KanR marker, a hSyn1 promoter, miRNA binding sites, and/or a PUC origin. For example, in some embodiments a coding sequence comprising at least 70%, at least 75%, at least 80%, at least 85%, at least 90%, or at least 95% identity to any one of SEQ ID NOs: 1-4 is expressed in a DNA construct. In some embodiments a coding sequence comprising the sequence of any one of SEQ ID NOs: 1-4 is expressed in a DNA construct. In some embodiments, the DNA construct includes one or more of elements such as a promoter, a 5′-ITR, a 3′-ITR, a WPRE, an hGH polyA, an F1 origin, a NeoR/KanR marker, a hSyn1 promoter, one or more miRNA binding sites and/or a PUC origin.

In some embodiments, an AAV vector associated with the disclosure includes a nucleic acid sequence encoding a MiniShank3 protein that comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or is 100% identical, including all values in between, to the sequence of SEQ ID NO: 21. In some embodiments, an AAV vector comprises a sequence corresponding to SEQ ID NO: 21, which encodes a MiniShank3 protein comprising the sequence of SEQ ID NO: 18.

In some embodiments, an AAV vector that includes a sequence that comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or is 100% identical, including all values in between, to the sequence of SEQ ID NO: 21, and which encodes a MiniShank3 protein comprising the sequence of SEQ ID NO: 18, may be delivered to a human subject in need thereof and may be suitable for treating a human subject who has a neurodevelopmental disorder.

As one of ordinary skill in the art would appreciate, any method known in the art for designing AAV vectors for clinical use, and for delivery of AAV vectors, may be compatible with aspects of the disclosure. For example, non-limiting examples of disclosure related to AAV vectors and delivery are provided in and incorporated by reference from U.S. Pat. No. 7,906,111, entitled “Adeno-associated virus (AAV) clades, sequences, vectors containing same, and uses therefor” and U.S. Pat. No. 9,834,788, entitled “AAV-vectors for use in gene therapy of choroideremia,” each of which is incorporated by reference herein in its entirety.

In some embodiments, an AAV vector associated with the disclosure includes a sequence encoding a MiniShank3 protein for AAV delivery that comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or is 100% identical, including all values in between, to the sequence of SEQ ID NO: 7 or 21, provided in Table 1. SEQ ID NO: 7 encodes the protein sequence of SEQ ID NO: 11. SEQ ID NO: 8 encodes the protein sequence of SEQ ID NO: 12. SEQ ID NO: 9 encodes the protein sequence of SEQ ID NO: 13. SEQ ID NO: 10 encodes the protein sequence of SEQ ID NO: 14. SEQ ID NO: 21 encodes the protein sequence of SEQ ID NO: 18.

In some embodiments, the AAV vector encoding a MiniShank3 protein for AAV delivery encodes a protein with a sequence that comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or is 100% identical, to any one of SEQ ID NOs: 11 or 17-20, provided in Table 1.

In some embodiments, the vector used for delivering the miniShank3 as disclosed herein can be a lentivirus vector. In some embodiments, the vector used for delivering the miniShank3 as disclosed herein can be an adenovirus vector.

In some embodiments, the vector construct disclosed herein can comprise SEQ ID NO: 21 as shown in Table 1.

In some embodiments, the vector comprising the polynucleotide of the Shank3 protein (i.e., the miniShank3 DNA construct) can be expressed in a specific tissue or cell of interest. In some embodiments, the vector disclosed herein comprises a promoter. In some embodiments, the vector comprises a cell-type specific promoter. In some embodiments, the promoter is a human promotor. In some embodiments, the human promoter is human Synapsin 1 (hSyn1). In some embodiments, the hSyn1promotor has a polynucleotide sequence corresponding to SEQ ID NO: 22. In some embodiments, the human promoter can be any promotor that is known in the art and is suitable for construction of the miniShank3. In some embodiments, the human promoter can be any promoter that has high specificity for neuronal tissues and cells. In some embodiments, the promoter can be a constitutive promoter. For example, the constitutive promoter can be a CAG promoter. As one of ordinary skill in the art would appreciate, any promoter may be used so long as the selected promoter is compatible with aspects of the disclosure.

### Compositions and Administration

The present disclosure provides compositions, including pharmaceutical compositions, comprising a polynucleotide (e.g., miniShank3) delivered in an AAV vector as disclosed herein and a pharmaceutically acceptable carrier.

The compositions of the disclosure may comprise an AAV alone, or in combination with one or more other viruses. In some embodiments, a composition comprises 1, 2, 3, 4, 5, 6, 7, 8, 9, 10, or more different AAVs each having one or more different Shank protein.

Suitable carriers may be readily selected by one of ordinary skill in the art in view of the indication for which the AAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure. Pharmaceutical compositions comprising AAV vectors are described further in U.S. Pat. No. 9,585,971 and US 2017/0166926, which are incorporated by reference herein in their entireties.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the AAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the AAV constructs disclosed herein. The formation and use of liposomes are generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the AAV vector may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In some embodiments, the pharmaceutical composition comprising a nucleic acid delivered in an AAV vector comprises other pharmaceutical ingredients, such as preservatives, or chemical stabilizers. Suitable exemplary preservatives include chlorobutanol, potassium sorbate, sorbic acid, sulfur dioxide, propyl gallate, parabens, ethyl vanillin, glycerin, phenol, thimerosal, and parachlorophenol. Suitable chemical stabilizers include gelatin and albumin. In many cases, it will be preferable to include isotonic agents, for example, sugars or sodium chloride. Prolonged absorption of the pharmaceutical compositions can be brought about by the use in the compositions of agents delaying absorption, for example, aluminum monostearate and gelatin.

The pharmaceutical forms suitable for delivering the AAV vectors include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi.

Methods described herein comprise administering AAV vector in sufficient amounts to transfect the cells of a desired tissue (e.g., brain) and to provide sufficient levels of gene transfer and expression without undue adverse effects. Conventional and pharmaceutically acceptable routes of administration include, but are not limited to, direct delivery to the selected organ, oral, inhalation, intraocular, intravenous including facial vein injection and retroorbital injection, intracerebroventricular (ICV), intramuscular, intrathecal, intracranial, subcutaneous, intradermal, intratumoral, and other parental routes of administration. Routes of administration may be combined, if desired. In some embodiments, the vector as disclosed herein is administered intravenously.

In some embodiments, the present disclosure provides methods of treating a subject having a neurodevelopmental disorder. In some embodiments, the present disclosure provides methods of treating a subject having an autism spectrum disorder (ASD). In some embodiments, the present disclosure provides methods of treating a subject having Phelan-McDermid syndrome. Methods provided herein, in some embodiments, comprise administering and delivering an effective amount of a composition comprising a vector that comprises a polynucleotide encoding a Shank3 protein (e.g., miniShank3) to a target environment or tissue of a subject. In some embodiments, the target tissue is cortex. In some embodiments, the target tissue is striatum. In some embodiments, the target tissue is thalamus cerebellum. In some embodiments, the target tissue is hippocampus. In some embodiments, the target tissue is any brain structure. In some embodiments, methods for administering and delivering an effective amount of a composition comprising a vector that comprises a polynucleotide encoding a Shank3 protein (e.g., miniShank3) to a target environment or tissue comprise delivering the composition to neurons or other brain cell types. In some embodiment, the vector is an AAV vector. In some embodiments, methods for delivering a nucleic acid to a target environment or tissue of a subject in need thereof comprise providing a composition comprising an AAV vector comprising at least a nucleic acid (e.g., miniShank3) to be delivered to the target environment or tissue of the subject and administering the composition to the subject. Methods of use of AAV vectors are described further in U.S. Pat. No. 9,585,971, US 2017/0166926, and WO2020/160337, which are incorporated by reference herein in their entireties. In some embodiments, the composition may comprise a capsid protein.

In some embodiments, the composition comprising a vector that comprises a polynucleotide encoding a Shank3 protein is delivered to the subject via intravenous administration, systemic administration, intracerebroventricular administration, in utero administration, intrathecal administration, retro-orbital injection, or facial vein injection. In some embodiments, in utero administration is used for a subject who is at the prenatal stage of development. In some embodiments, the composition is delivered to a subject via a nanoparticle. In some embodiments, the composition is delivered to a subject via a viral vector. In some embodiments, the composition is delivered to a subject via any carriers suitable for delivering nucleic acid materials.

Any composition comprising a vector that comprises a polynucleotide encoding a protein that would be of some use or benefit to the subject may be delivered to a target environment or tissue of the subject according to methods disclosed herein

In addition to the methods of delivery described above, the following techniques are also contemplated as alternative methods of delivering the AAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

The dose of AAV comprising a polynucleotide that encodes a Shank3 protein (e.g., miniShank3) required to achieve a particular “therapeutic effect,” e.g., the units of dose in absolute vector genomes (vg) or vector genomes per milliliter of pharmaceutical solution (vg/mL) will vary based on several factors including, but not limited to: the route of AAV administration, the level of gene expression required to achieve a therapeutic effect, the specific disease or disorder being treated, and the stability of the gene product. Doses that give maximal percentage of infection without affecting neurodevelopment are also suitable. One of skill in the art can readily determine a AAV dose range to treat a patient having a particular disease or disorder based on the aforementioned factors, as well as other factors.

An effective amount of AAV vector is an amount sufficient to infect an animal or human subject or target a desired tissue. The effective amount will depend primarily on factors such as the species, age, gender, weight, health of the subject, and the tissue to be targeted, and may thus vary among subjects and tissues. The term “effective amount” or “amount effective” in the context of a composition or dose for administration to a subject refers to an amount of the composition or dose that produces one or more desired responses in the subject. In some embodiments, an effective amount of a composition disclosed herein may partially or fully rescue the effects of a mutated Shank3 gene and/or partially or fully restore loss of function of the Shank3 protein. An effective amount can involve reducing the level of an undesired response, although in some embodiments, it involves preventing an undesired response altogether. An effective amount can also involve delaying the occurrence of an undesired response. An effective amount can also be an amount that produces a desired therapeutic endpoint or a desired therapeutic result. In other embodiments, the amounts effective can involve enhancing the level of a desired response, such as a therapeutic endpoint or result. The achievement of any of the foregoing can be monitored by routine methods and the methods as disclosed in the present application. Effective amounts will depend, of course, on the particular subject being treated; the severity of a condition; the individual patient parameters including age, physical condition, size and weight; the duration of the treatment; the nature of concurrent therapy (if any); the specific route of administration and like factors.

For example, in some embodiments, the number of vector genomes administered to the subject is any value between about 6.0×10¹¹ vg and about 9.0×10¹³ vg. In some embodiments, the number of vector genomes administered to the subject is any value between about 6.0×10¹³ vg/mL and about 9.0×10¹³ vg. In some embodiments, the number of vector genomes administered to the subject is any value between about 1×10¹⁰ to about 1×10¹² vg. In certain embodiments, the effective amount of AAV is 10¹⁰, 10¹¹, 10¹², 10¹³, or 10¹⁴ genome copies per kg. In certain embodiments, the effective amount of AAV is 10¹⁰, 10¹¹, 10¹², 10¹³, 10¹⁴, or 10¹⁵ genome copies per subject. In some cases, a dosage between about 10¹¹ to 10¹³ AAV genome copies is appropriate. In some embodiments, the number of vector genomes administered to the subject can be any dose that is suitable for the treatments and methods disclosed herein.

In some embodiments, a dose of AAV is administered to a subject no more than once per calendar day (e.g., a 24-hour period). In some embodiments, a dose of AAV is administered to a subject no more than once per 2, 3, 4, 5, 6, or 7 calendar days. In some embodiments, a dose of AAV is administered to a subject no more than once per calendar week (e.g., 7 calendar days). In some embodiments, a dose of AAV is administered to a subject no more than bi-weekly (e.g., once in a two calendar week period). In some embodiments, a dose of AAV is administered to a subject no more than once per calendar month (e.g., once in 30 calendar days). In some embodiments, a dose of AAV is administered to a subject no more than once per six calendar months. In some embodiments, a dose of AAV is administered to a subject no more than once per calendar year (e.g., 365 days or 366 days in a leap year). In some embodiments, a dose of rAAV is administered to a subject no more than once per two calendar years (e.g., 730 days or 731 days in a leap year). In some embodiments, a dose of AAV is administered to a subject no more than once per three calendar years (e.g., 1095 days or 1096 days in a leap year).

Formulation of pharmaceutically-acceptable excipients and carrier solutions disclosed herein is well-known to those of skill in the art, as is the development of suitable dosing and treatment regimens for using the particular compositions described herein in a variety of treatment regimens. Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Expression of Proteins Associated with the Shank Protein Network

Methods and compositions provided herein, in some embodiments, are useful for treating a neurodevelopmental disorder, such as, for example, an autism spectrum disorder (ASD), or Phelan-McDermid syndrome. As disclosed in and incorporated by reference from PCT Publication No. WO2022/040239 and US Patent Publication No. US2023/0340041, delivery of a miniShank3 via a viral vector such as AAV vector in a mouse model is effective in restoring functionality of postsynaptic density (PSD) proteins. In some embodiments, expression levels of PSD proteins are used to evaluate the efficacy of the administration of the miniShank3. In some embodiments, the PSD protein is Homer. In some embodiments, the PSD protein is post-synaptic density protein 95 (PSD95). In some embodiments, the PSD protein is SynGap1. In some embodiments, the PSD protein is SAPAP3. In some embodiments, the PSD protein is NR1. In some embodiments, the PSD protein is NR2B. In some embodiments, the PSD protein is GluR2. In some embodiments, the PSD protein is any protein that can be improved or restored upon the miniShank3 treatment.

In some embodiments, an increase of any of the PSD proteins, as compared to an untreated control subject, may indicate efficacy of the miniShank3. Methods for detecting gene expression and protein levels are well-known in the art.

In some embodiments, expression of Homer in the subject after treated with the miniShank3 is increased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control. In some embodiments, expression of post-synaptic protein (PSD95) in the subject after treated with the miniShank3 is increased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control. In some embodiments, expression of SynGap1 in the subject after treated with the miniShank3 is increased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control. In some embodiments, expression of SAPAP3 in the subject after treated with the miniShank3 is increased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control. In some embodiments, expression of NR1 in the subject after treated with the miniShank3 is increased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control. In some embodiments, expression of NR2B in the subject after treated with the miniShank3 is increased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control. In some embodiments, expression of GluR2 in the subject after treated with the miniShank3 is increased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control.

In some embodiments, administration of a MiniShank3 or a composition comprising a MiniShank3 can lead to improving sleep efficiency. In some embodiments, a subject has improved sleep efficiency after being administered an effective amount of a composition comprising an expression construct comprising a polynucleotide encoding a Shank protein such as a MiniShank3 protein. In some embodiments, the sleep efficiency in the subject after being administered to an effective amount of the composition is increased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control subject. Improved sleep efficiency includes less sleep disturbance, which includes but is not limited to having trouble falling and staying asleep. Measurement of sleep efficiency can be conducted using any methods known in the art.

In some embodiments, administration of a MiniShank3 or a composition comprising a MiniShank3 can lead to improving social impairment. In some embodiments, the social impairment of the subject is improved after being administered an effective amount of a composition comprising an expression construct comprising a polynucleotide encoding a Shank protein such as a MiniShank3 protein. In some embodiments, the social impairment in the subject after being administered to an effective amount of the composition is decreased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control subject. Measurement of social impairment can be conducted using any methods known in the art.

As used herein, “social impairment” refers to behavioral abnormalities or defects that prohibit a subject from displaying voluntary social interaction.

In some embodiments, administration of a MiniShank3 or a composition comprising a MiniShank3 can lead to improving locomotion and/or motor coordination deficits. In some embodiments, the locomotion and/or motor coordination deficits of the subject are improved after being administered an effective amount of a composition comprising an expression construct comprising a polynucleotide encoding a Shank protein such as a MiniShank3 protein. In some embodiments, the locomotion and/or motor coordination deficits in the subject after being administered to an effective amount of the composition is decreased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control subject. Measurement of locomotion and/or motor coordination deficits can be conducted using any methods known in the art.

As used herein, “locomotion and/or motor coordination deficits” can include, for example, lack of coordination, loss of balance, and/or a shuffling gait.

In some embodiments, administration of a MiniShank3 or a composition comprising a MiniShank3 can lead to improvement in cortical-striatal synaptic dysfunction. In some embodiments, the cortical-striatal synaptic dysfunction of the subject are improved after being administered an effective amount of a composition comprising an expression construct comprising a polynucleotide encoding a Shank protein such as a MiniShank3 protein. In some embodiments, the cortical-striatal synaptic dysfunction in the subject after being administered to an effective amount of the composition is decreased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control subject. Measurement of cortical-striatal synaptic dysfunction can be conducted using any methods known in the art.

As used herein, “cortical-striatal synaptic dysfunction” refers to defective corticostriatal circuits in the brain that can cause repetitive and compulsive behaviors, such as in neuropsychiatric disorders and neurodevelopmental diseases such as autism, obsessive-compulsive disorders, and Tourette syndrome.

Some aspects of the technology described herein may be understood further based on the non-limiting illustrative embodiments described in the below Examples section. Any limitations of the embodiments described in the below Examples section are limitations only of the embodiments described in the below Examples section and are not limitations of any other embodiments described herein.

### EXAMPLES

In order that the invention described herein may be more fully understood, the following examples are set forth. The examples described in this application are offered to illustrate the systems and methods provided herein and are not to be construed in any way as limiting their scope.

### Example 1: Behavioral Characterization and Gene Therapy Trial of Shank3 Mutant Marmosets

The goal of this project is to develop a platform for studying neuropsychiatric disorders in nonhuman primates and testing the effectiveness of treatments. This should serve to focus human trials on the most promising options. This effort was begun by establishing a monogenic Shank3 ASD model of mutant marmosets. Natural breeding was used to produce 16 young Shank3+/− marmosets. A wide range of assays was designed to study the animals' natural social behavior, vocalizations, cognition, responses to a variety of stimuli, gait, and sleep quality. By comparing over 100 control animals with the 16 Shank3+/− marmosets, it was determined how their behaviors differed across all these assays. The Shank3+/− marmosets had an increased fear of threatening stimuli, decreased interest in social stimuli, performed worse on cognitive tasks requiring flexibility, and atypical social interactions. Based on these results, a gene replacement therapy method was tested using an AAV injected via IV to express miniShank3 in neurons across the brain.

For an ASD therapy to be useful, it should be able to reverse the negative effects of the disorder after they become detectable. Results from the mouse model suggested that this could be possible, so a similar approach was pursued in the marmosets. 4 Shank3+/− marmosets were randomly selected to receive gene therapy injections at 3 months old (approximately 3 years of human age) and 4 Shank3+/− marmosets to receive gene therapy injections at 9 months old (approximately 10 years of human age), with the remaining 8 Shank3+/− marmosets as untreated controls. Importantly, the phenotypes associated with the mutation are already observable at 6 months old. It was reasoned that the 3-month time point represents an “early” intervention and the 9-month time point represents a “late” intervention during development to see whether we can reverse the phenotypes.

Shank3+/− animals which received gene therapy injections were found to have reduced fear of threatening stimuli, had increased interest in social stimuli, performed better on certain cognitive tasks, and had more typical social interactions.

Based on these results, two new directions will be pursued, including: 1) Testing circuit-based drug treatments as an alternative to gene therapy to determine whether this could provide a treatment option for polygenic ASD; and 2) Using a range of neurophysiological methods (fMRI, fUS, and eCOG) to study the differences in brain circuit activity associated with the Shank3+/− animals and investigating how the gene therapy affected those measures.

This study will help to establish that the marmoset can serve as a model for neuropsychiatric disorders. In the future, similar screening methods can be used as a platform for studying therapies for a wide range of disorders such as schizophrenia, depression and Alzheimer's disease.

A goal of this study was to determine if Shank3+/− marmosets exhibit ASD phenotypes and if these phenotypes were reverted by gene therapy. The behavioral phenotypes tested were atypical behavior (restricted/repetitive behavior, unusual patterns of attention, unlike others), atypical social behavior (either uninterested, not understanding, or disliking social interactions), cognitive deficits (ASD often associated with severe intellectual disability), and abnormal connectivity (difference in resting-state fMRI scan or task-related fMRI scan).

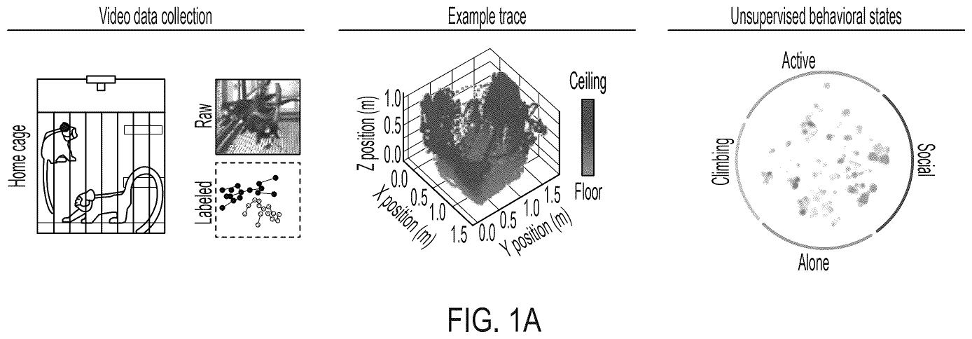

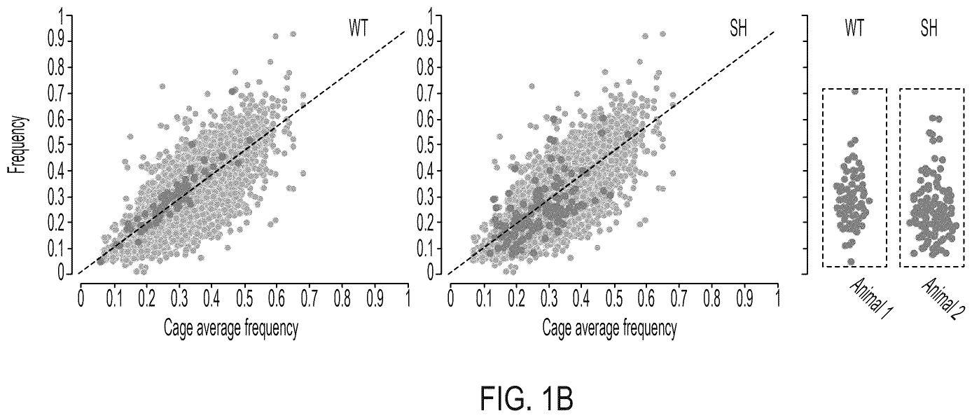

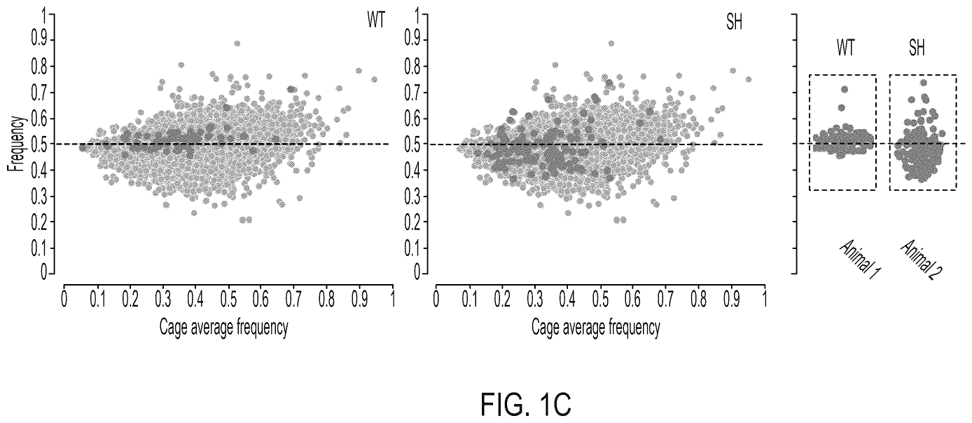

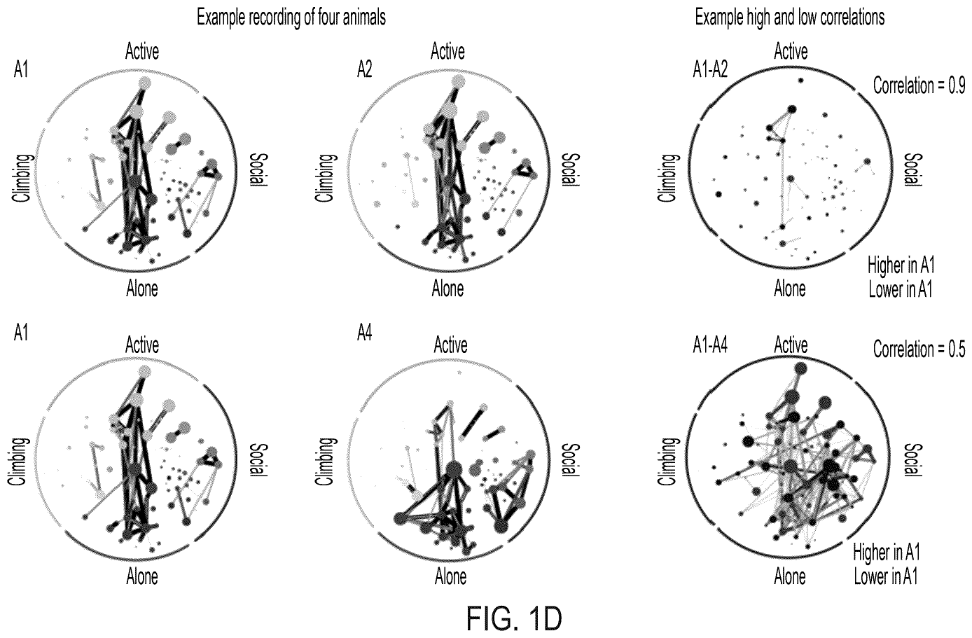

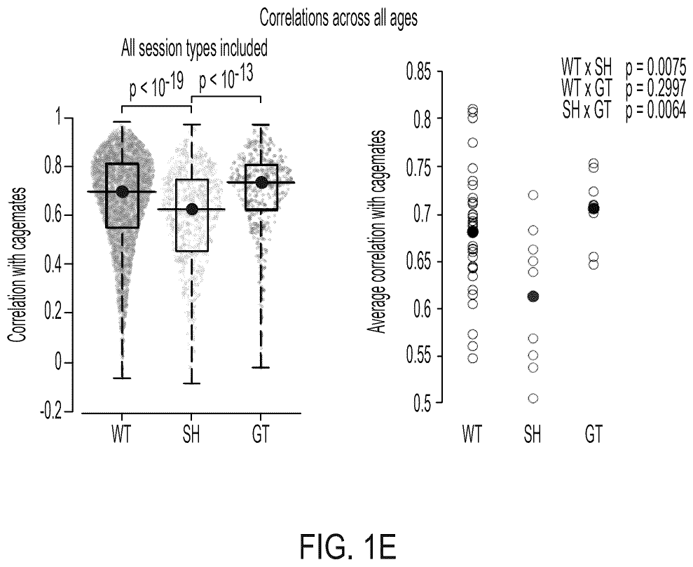

FIGS. 1A-1E show that Shank3+/− marmoset exhibit atypical behavior measured by unsupervised behavioral states (e.g., active, social, alone, and climbing) and gene therapy delivery of miniShank3 (SEQ ID NO: 21) reverses this behavior.

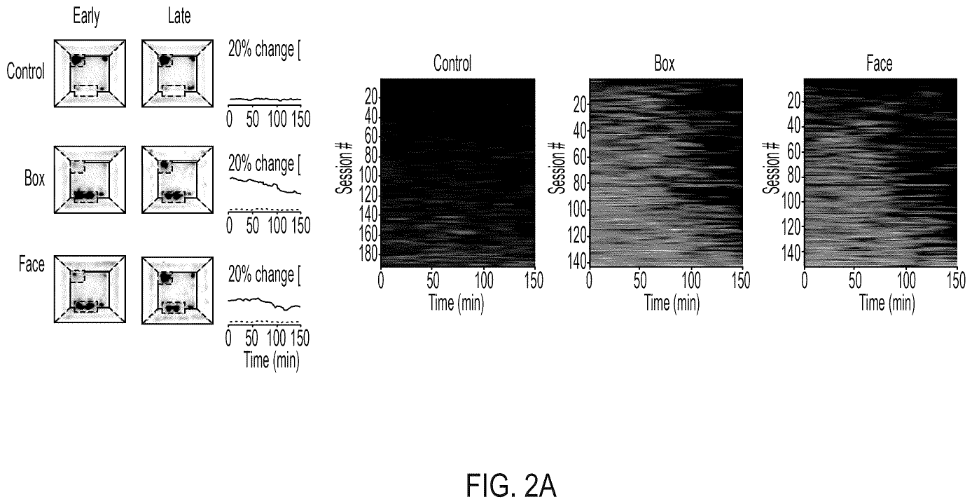

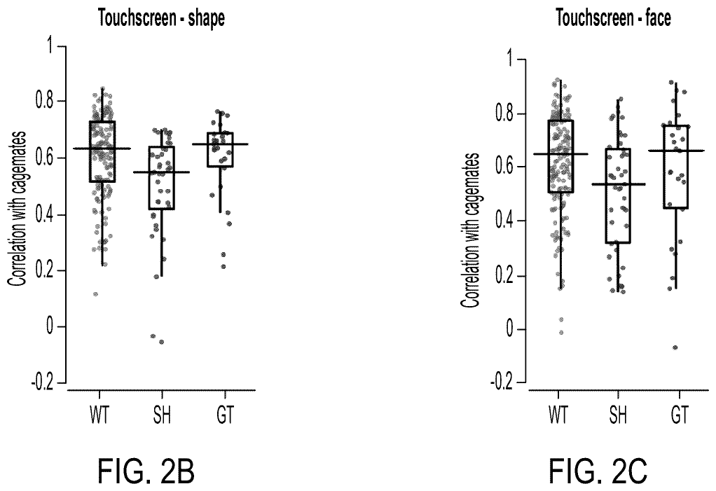

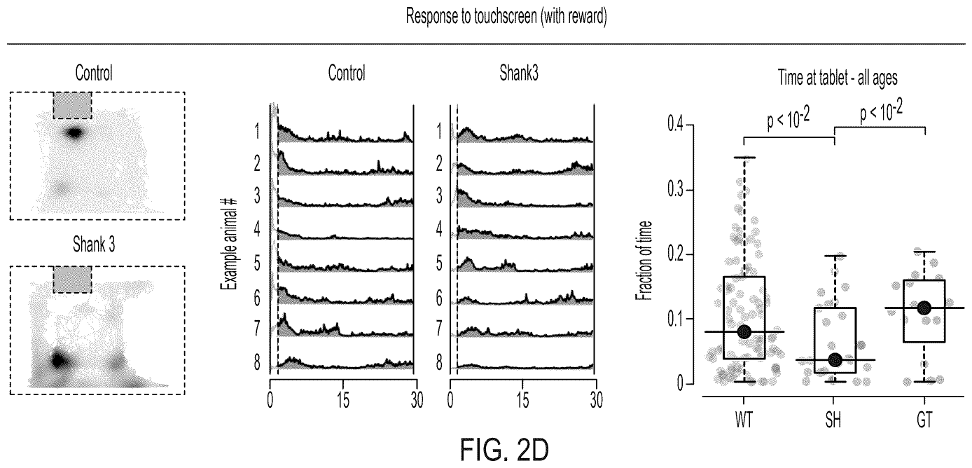

FIGS. 2A-2D show that Shank3+/− marmoset exhibit atypical behavior measured by touchscreen-based behavioral stimuli and gene therapy delivery of miniShank3 (SEQ ID NO: 21) reverses this behavior.

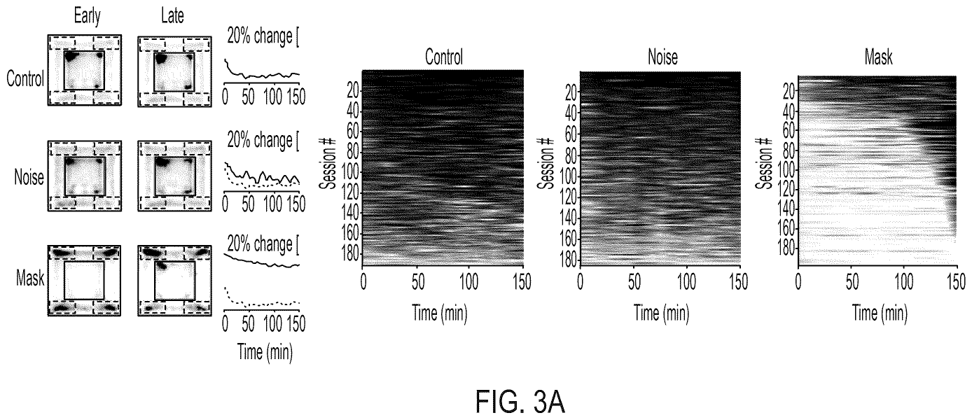

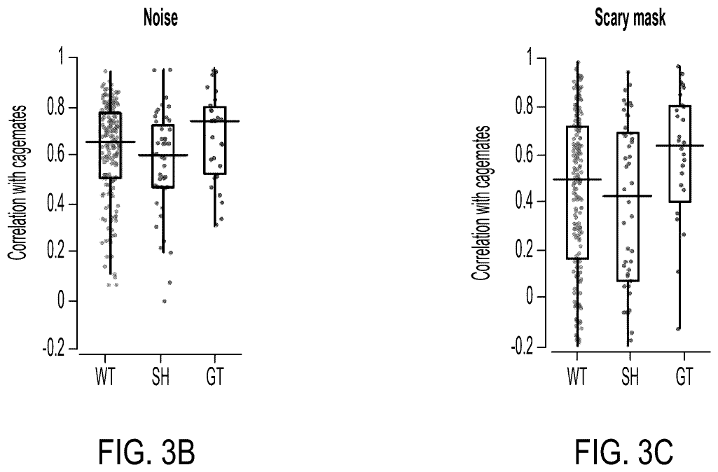

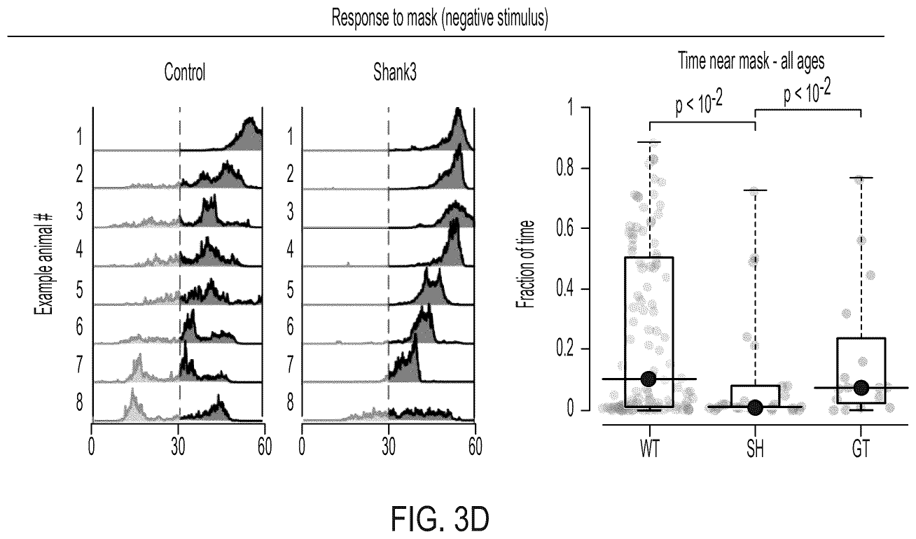

FIGS. 3A-3D show that Shank3+/− marmoset exhibit atypical behavior measured by negative valence behavioral stimuli and gene therapy delivery of miniShank3 (SEQ ID NO: 21) reverses this behavior.

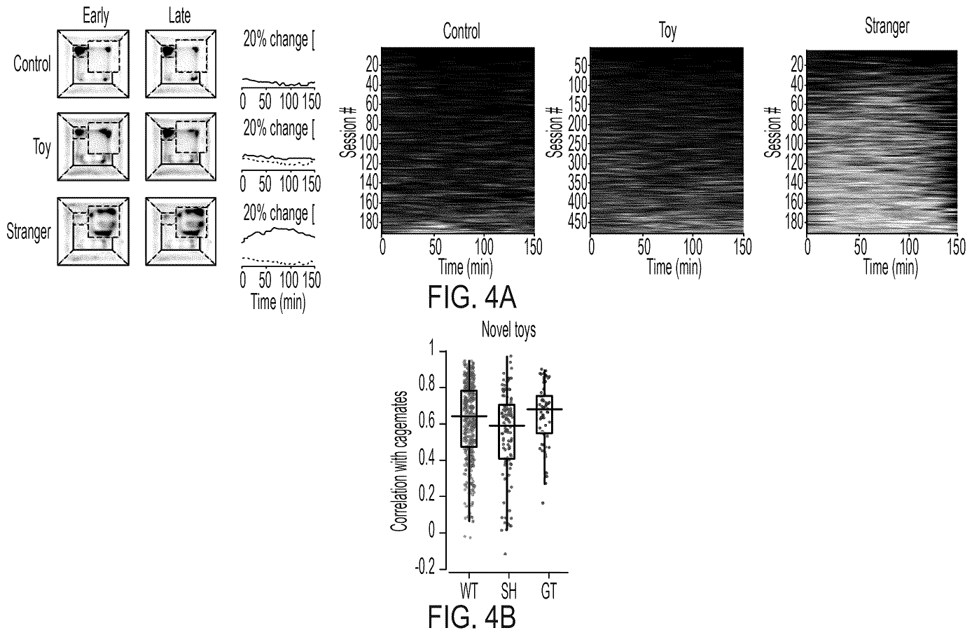

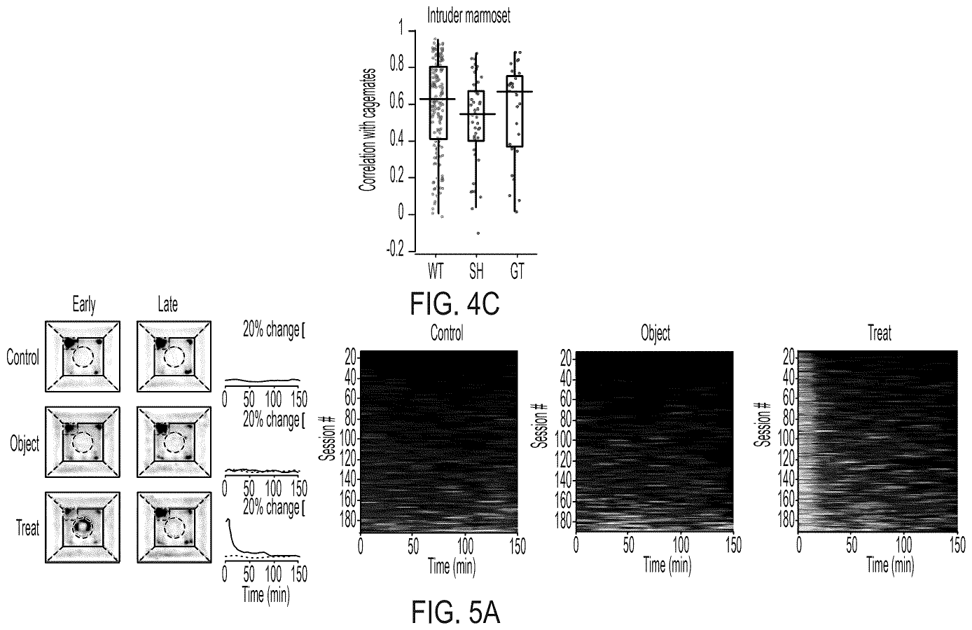

FIGS. 4A-4C show that Shank3+/− marmoset exhibit atypical behavior measured by social behavioral stimuli and gene therapy delivery of miniShank3 (SEQ ID NO: 21) reverses this behavior.

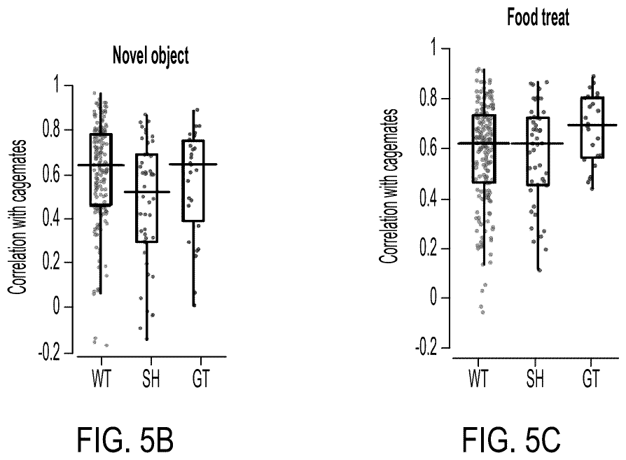

FIGS. 5A-5C show that Shank3+/− marmoset exhibit atypical behavior measured by positive valence behavioral stimuli and gene therapy delivery of miniShank3 (SEQ ID NO: 21) reverses this behavior.

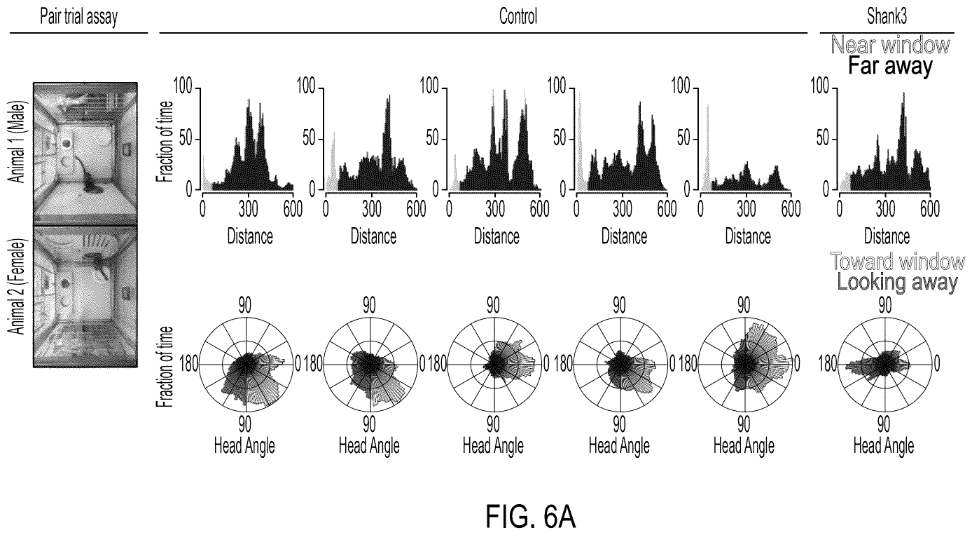

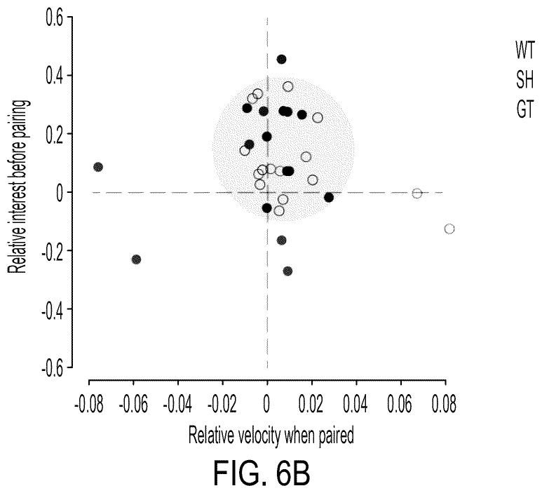

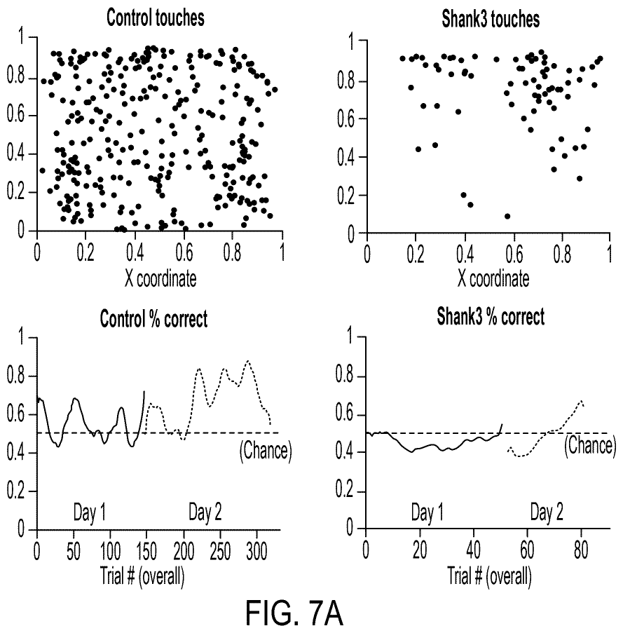

FIGS. 6A-6B show that Shank3+/− marmoset exhibit atypical social behavior measured by novel animal studies and gene therapy delivery of miniShank3 (SEQ ID NO: 21) reverses this behavior.

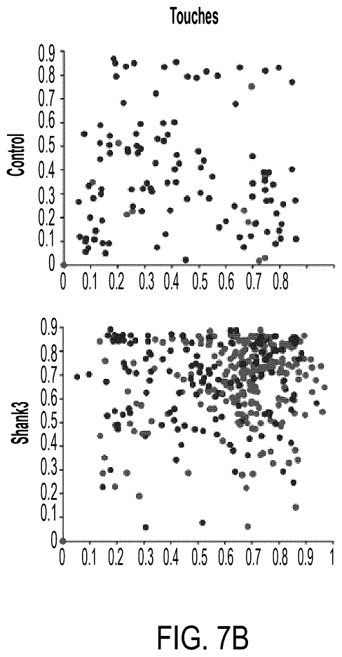

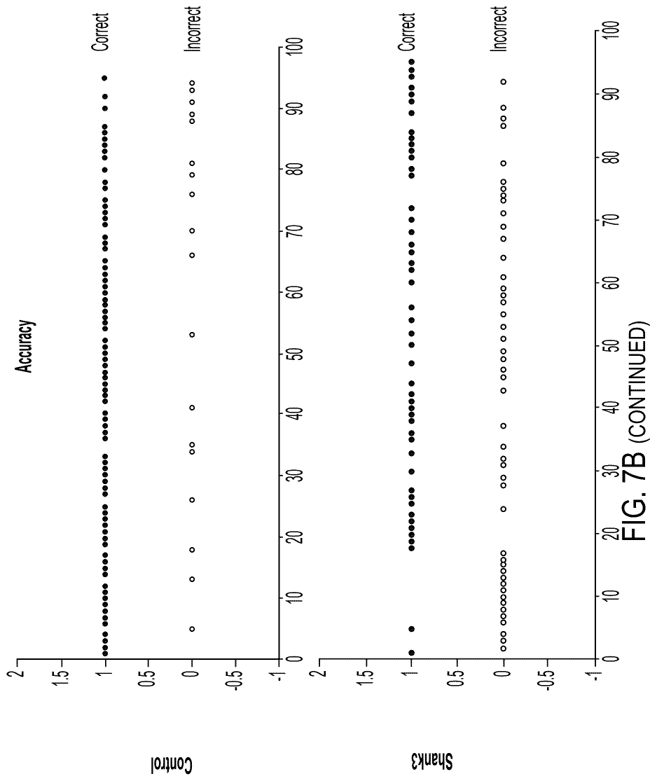

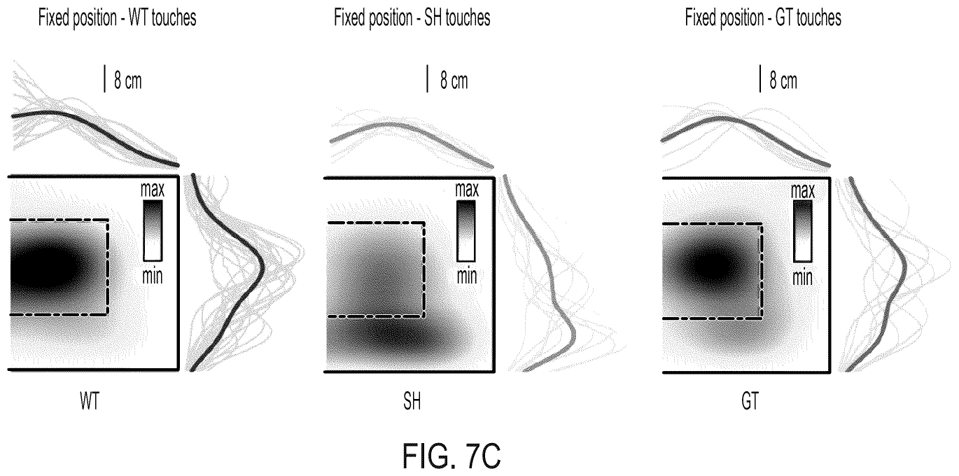

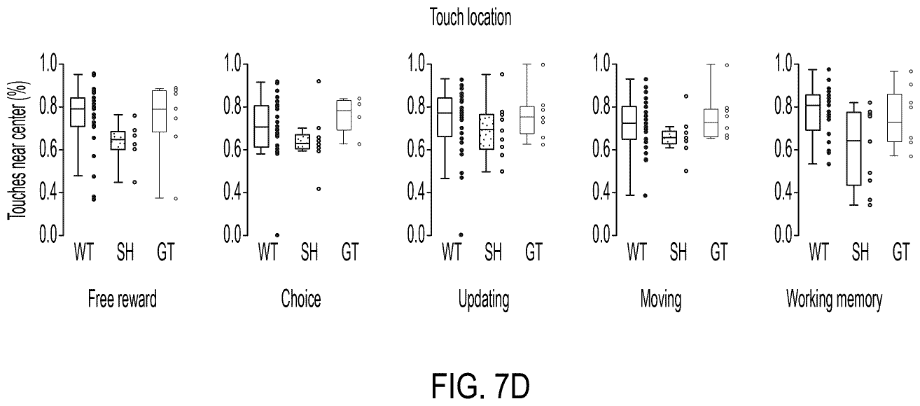

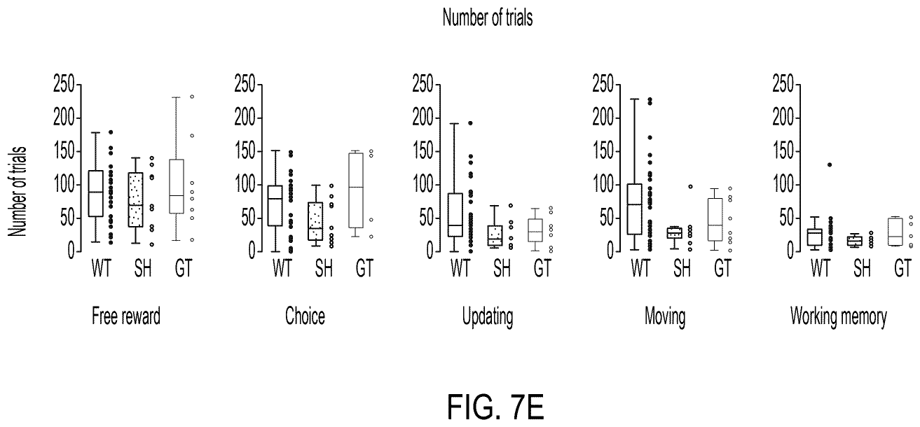

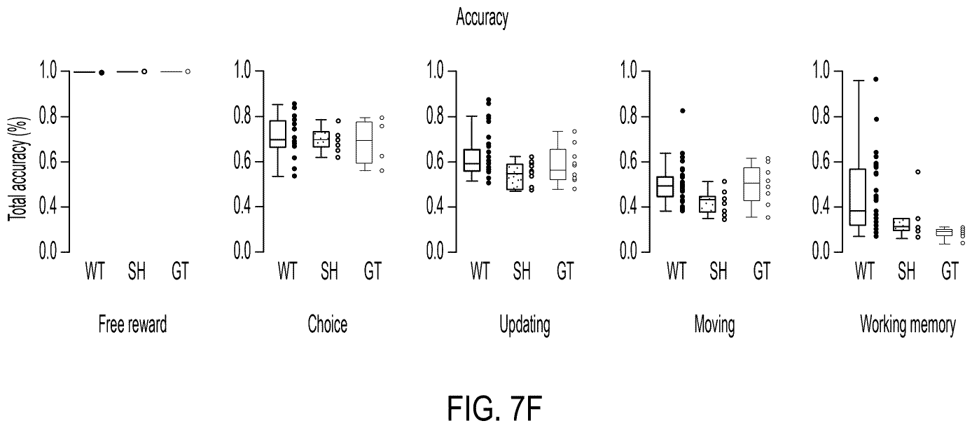

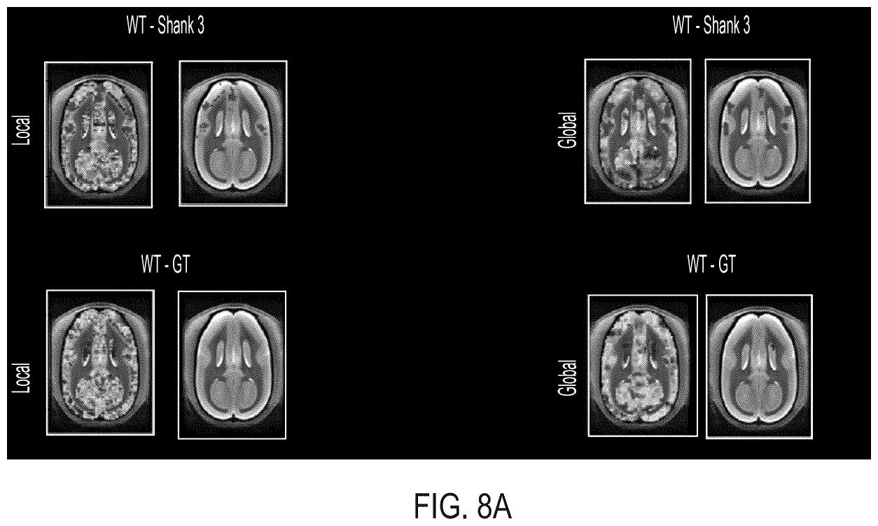

FIGS. 7A-7F show that Shank3+/− marmoset exhibit cognitive deficit measured by cognitive task studies and gene therapy delivery of miniShank3 (SEQ ID NO: 21) reverses this cognitive deficit.

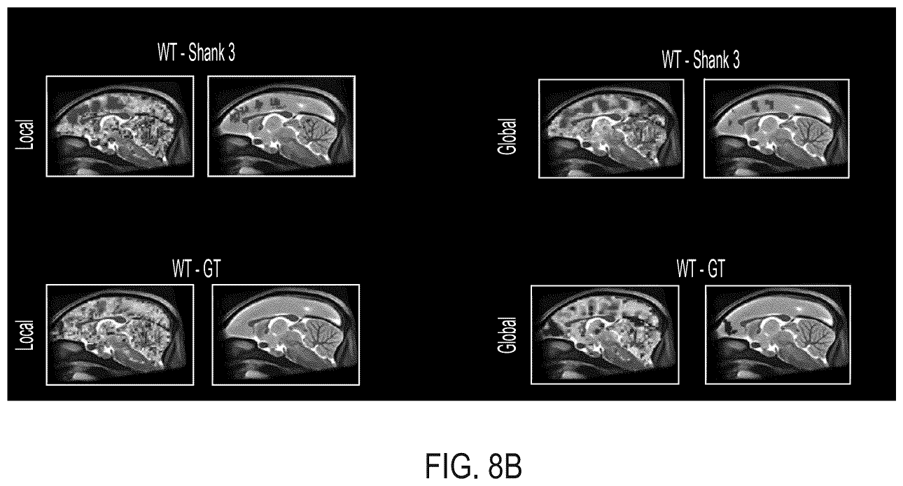

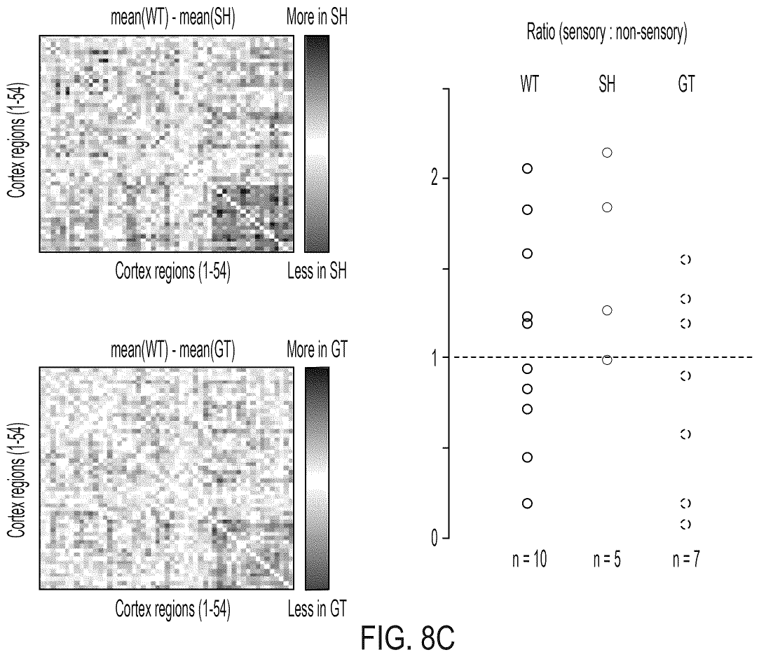

FIGS. 8A-8C show that Shank3+/− marmoset exhibit abnormal functional connectivity measured by fMRI and gene therapy delivery of miniShank3 (SEQ ID NO: 21) reverses this cognitive deficit.

TABLE 1
Mouse and Human miniShank3 Sequences and Vector Sequences

| SEQ | | |
|---|---|---|
| ID | | |
| NO: | Description | Sequence |
| 1 | Mouse | atgggcccgaagcggaaactttacagtgccgtccccggccgcaagttcatcgctgtgaaggcgcacagcc |
| | miniShank3- | cgcagggcgagggcgagatcccgctgcaccgcggcgaggccgtgaaggtgctcagcattggggaggg |
| | V1 | cggtttctgggagggaaccgtgaagggccgaacaggctggttcccagctgactgtgtggaagaagtgcag |
| | | atgcgacagtatgacacccggcatgaaaccagagaggaccggacgaagcgtctcttccgccactacactg |
| | | tgggttcctatgacagcctcacttcacacagcgattatgtcatcgatgataaggtggctatcctgcagaaaag |
| | | ggaccatgaggggtttggctttgttctccggggagccaaagcagagacccccattgaggagtttacaccca |
| | | cacctgccttccctgcactccaataccttgagtctgtagatgtggaaggtgtggcctggagggctggacttcg |
| | | aactggggacttcctcattgaggtgaacggagtgaatgtcgtgaaggttggacacaagcaagtggtgggtct |
| | | catccgtcagggtggcaaccgcctggtcatgaaggttgtgtctgtgaccaggaaacccgaggaggatggt |
| | | gctcggcgcagagccccaccacccccaaagagggctcccagcaccacgctgaccctgcggtccaagtcc |
| | | atgacggctgagctcgaggaacttgcttccattcggagctcagaggaggagccagagctggtattcgctgt |
| | | gaacctgccacctgctcagctgtcctccagcgatgaggagaccagagaggagctggcccgcatagggct |
| | | agtgccaccccctgaagagtttgccaatgggatcctgctgaccaccccgcccccagggccgggccccttg |
| | | cccaccacggtacccagcccggcctcagggaagcccagcagcgagctgccccctgcccctgagtctgca |
| | | gctgactctggagtagaggaggctgacactcgaagctccagtgacccccacctggagaccacaagcacc |
| | | atttccacagtgtccagcatgtccaccctgagctcggagagtggggaactcacggacacccacacctccttt |
| | | gccgatggacacacttttctactcgagaagccaccagtgcctcccaagcccaagctcaagtccccgctggg |
| | | gaaggggccggtgaccttcagggacccgctgctgaagcaatcctcggacagtgagctcatggcccagca |
| | | gcaccatgctgcctctactgggttggcttctgctgctgggcccgcccgccctcgctacctcttccagagaag |
| | | gtccaagctgtggggggaccccgtggagagtcgggggctccctgggcctgaagatgacaaaccaactgt |
| | | gatcagtgagctcagctcccgtctgcagcagctgaataaagacacacgctccttgggggaggaaccagttg |
| | | gtggcctgggcagcctgctggaccctgctaagaagtcacccattgcagcagctcggctcttcagcagcctc |
| | | ggtgagctgagcaccatctcagcgcagcgcagcccggggggcccgggcggaggggcctcctactcggt |
| | | gcggcccagcggccggtaccccgtggcgagacgagccccgagcccagtgaaacccgcatcgctggag |
| | | cgggtggaggggctgggggcgggcgtgggaggcgcggggcggcccttcggcctcacgcctcccacca |
| | | tcctcaagtcgtccagcctctccatcccgcacgaacccaaggaagtgcgcttcgtggtgcgaagtgtgagtg |
| | | cgcgcagccgctccccctcaccatctccgctgccctcgccttctcccggctctggccccagtgccggcccg |
| | | cgtcggccatttcaacagaagcccctgcagctctggagcaagttcgatgtgggcgactggctggagagcat |
| | | ccacttaggcgagcaccgagaccgcttcgaggaccatgagatcgaaggcgcacacctgcctgcgctcac |
| | | caaggaagacttcgtggagctgggcgtcacacgcgttggccaccgcatgaacatcgagcgtgcgctcagg |
| | | cagctggatggcagctga |
| 2 | Human | atgggcccgaagcggaaactttacagcgccgtccccggccgcaagttcatcgccgtgaaggcgcacagc |
| | miniShank3- | ccgcagggtgaaggcgagatcccgctgcaccgcggcgaggccgtgaaggtgctcagcattggggaggg |
| | V1 | cggtttctgggagggaaccgtgaaaggccgcacgggctggttcccggccgactgcgtggaggaagtgca |
| | | gatgaggcagcatgacacacggcctgaaacgcgggaggaccggacgaagcggctctttcggcactacac |
| | | agtgggctcctacgacagcctcacctcacacagcgattatgtcattgatgacaaagtggctgtcctgcagaa |
| | | acgggaccacgagggctttggttttgtgctccggggagccaaagcagagacccccatcgaggagttcacg |
| | | cccacgccagccttcccggcgctgcagtatctcgagtcggtggacgtggagggtgtggcctggagggcc |
| | | gggctgcgcacgggagacttcctcatcgaggtgaacggggtgaacgtggtgaaggtcggacacaagcag |
| | | gtggtggctctgattcgccagggtggcaaccgcctcgtcatgaaggttgtgtctgtgacaaggaagccaga |
| | | agaggacggggctcggcgcagagccccaccgccccccaagagggcccccagcaccacactgaccctg |
| | | cgctccaagtccatgacagctgagctcgaggaacttgcctccattcggagctcagaggaagagccagagc |
| | | tggtgtttgctgtgaacctgccacctgcccagctgtcgtccagcgatgaggagaccagggaggagctggcc |
| | | cgaattgggttggtgccaccccctgaagagtttgccaacggggtcctgctggccaccccactcgctggccc |
| | | gggcccctcgcccaccacggtgcccagcccggcctcagggaagcccagcagtgagccaccccctgccc |
| | | ctgagtctgcagccgactctggggtggaggaggctgacacacgcagctccagcgacccccacctggaga |
| | | ccacaagcaccatctccacggtgtccagcatgtccaccttgagctcggagagcggggaactcactgacac |
| | | ccacacctccttcgctgacggacacacttttctactcgagaagccaccagtgcctcccaagcccaagctcaa |
| | | gtccccgctggggaaggggccggtgaccttcagggacccgctgctgaagcagtcctcggacagcgagct |
| | | catggcccagcagcaccacgccgcctctgccgggctggcctctgccgccgggcctgcccgccctcgcta |
| | | cctcttccagagaaggtccaagctatggggggaccccgtggagagccgggggctccctgggcctgaaga |
| | | cgacaaaccaactgtgatcagtgagctcagctcccgcctgcagcagctgaacaaggacacgcgttccctg |
| | | ggggaggaaccagttggtggcctgggcagcctgctggaccctgccaagaagtcgcccatcgcagcagct |
| | | cggctcttcagcagcctcggtgagctgagctccatttcagcgcagcgcagccccgggggcccgggggc |
| | | ggggcctcgtactcggtgaggcccagtggccgctaccccgtggcgagacgcgccccgagcccggtgaa |
| | | gcccgcgtcgctggagcgggtggaggggctggggggggcgcggggggcgcagggcggcccttcgg |
| | | cctcacgccccccaccatcctcaagtcgtccagcctctccatcccgcacgagcccaaggaggtgcgcttcg |
| | | tggtgcgcagcgtgagcgcgcgcagtcgctccccctcgccgtcgccgctgccctcgcccgcgtccggcc |
| | | ccggccccggcgcccccggcccacgccgacccttccagcagaagccgctgcagctctggagcaagttcg |
| | | acgtgggcgactggctggagagcatccacctaggcgagcaccgcgaccgcttcgaggaccatgagatag |
| | | aaggcgcgcacctacccgcgcttaccaaggacgacttcgtggagctgggcgtcacgcgcgtgggccacc |
| | | gcatgaacatcgagcgcgcgctcaggcagctggacggcagctga |
| 3 | Mouse | atggacggccccggggccagcgccgtggtcgtgcgcgtcggcatcccggacctgcaacaaacgaagtg |
| | miniShank3- | cctgcgtctggacccaaccgcgcccgtgtgggccgccaagcagcgtgtgctctgcgccctcaatcatagc |
| | V2 | cttcaagacgcgctcaactacgggctattccagcctccctcccggggtcgcgccggcaagttcctggatga |
| | | agagcggctcttacaggactacccgcctaacctggacacgcccctgccctatctggagttccgatacaagc |
| | | ggagagtttatgcccagaacctcatagatgacaagcagtttgcaaagctacacacaaaggcaaacctgaag |
| | | aagttcatggactatgtccagctacacagcacagataaggggcccgcctgctggacaaggggctggacc |
| | | ccaatttccatgaccctgactcaggagagtgccctctgagccttgcggcacagttggacaacgccactgacc |
| | | tcctgaaggttctccgcaacggcggtgctcatctggacttccggacccgagatgggctgacagccgtccac |
| | | tgtgctacccgccagcggaacgcaggggcattgacgaccctgctggacctgggggcttcgcctgactaca |
| | | aggacagccgcggcctgacgcccctgtaccatagtgccctagggggggggatgccctctgttgcgagct |
| | | gcttctccatgatcatgcacagctggggaccactgatgagaatggttggcaagagatccatcaggcctgtcg |
| | | ctttggacacgtgcagcacctggagcaccttttgttctatggggccaacatgggtgctcagaatgcctcggg |
| | | aaacacagccctgcacatctgtgccctctacaaccaggagagttgcgcgcgcgtcctgcttttccgtggtgc |
| | | caacaaggacgtccgcaattacaacagccagacagccttccaggtggccattattgcagggaactttgagct |
| | | tgccgaggtaatcaagacccacaaagactccgatgtcgtaccattcagggaaacccccagctatgcaaagc |
| | | gacggcgtctggctggcccgagtggcctggcatccccacggcccttacagcgctcagccagtgatatcaa |
| | | cctgaaaggtgcgccgccgccccggggcccgaagcggaaactttacagtgccgtccccggccgcaagtt |
| | | catcgctgtgaaggcgcacagcccgcagggcgagggcgagatcccgctgcaccgcggcgaggccgtg |
| | | aaggtgctcagcattggggagggcggtttctggggggaaccgtgaagggccgaacaggctggttccca |
| | | gctgactgtgtggaagaagtgcagatgcgacagtatgacacccggcatgaaaccagagaggaccggacg |
| | | aagcgtctcttccgccactacactgtgggttcctatgacagcctcacttcacacagcgattatgtcatcgatgat |
| | | aaggtggctatcctgcagaaaagggaccatgaggggtttggctttgttctccggggagccaaagcagagac |
| | | ccccattgaggagtttacacccacacctgccttccctgcactccaataccttgagtctgtagatgtggaaggtg |
| | | tggcctggagggctggacttcgaactggggacttcctcattgaggtgaacggagtgaatgtcgtgaaggttg |
| | | gacacaagcaagtggtgggtctcatccgtcagggtggcaaccgcctggtcatgaaggttgtgtctgtgacc |
| | | aggaaacccgaggaggatggtgctcggcgcagagccccaccacccccaaagagggctcccagcacca |
| | | cgctgaccctgcggtccaagtccatgacggctgagctcgaggaacttgcttccattcggagctcagaggag |
| | | gagccagagctggtattcgctgtgaacctgccacctgctcagctgtcctccagcgatgaggagaccagaga |
| | | ggagctggcccgcatagggctagtgccaccccctgaagagtttgccaatgggatcctgctgaccaccccg |
| | | cccccagggccgggccccttgcccaccacggtacccagcccggcctcagggaagcccagcagcgagct |
| | | gccccctgcccctgagtctgcagctgactctggagtagaggaggctgacactcgaagctccagtgacccc |
| | | cacctggagaccacaagcaccatttccacagtgtccagcatgtccaccctgagctcggagagtggggaact |
| | | cacggacacccacacctcctttgccgatggacacacttttctactcgagaagccaccagtgcctcccaagcc |
| | | caagctcaagtccccgctggggaaggggccggtgaccttcagggacccgctgctgaagcaatcctcgga |
| | | cagtgagctcatggcccagcagcaccatgctgcctctactgggttggcttctgctgctgggcccgcccgcc |
| | | ctcgctacctcttccagagaaggtccaagctgtggggggaccccgtggagagtcgggggctccctgggcc |
| | | tgaagatgacaaaccaactgtgatcagtgagctcagctcccgtctgcagcagctgaataaagacacacgct |
| | | ccttgggggaggaaccagttggtggcctgggcagcctgctggaccctgctaagaagtcacccattgcagc |
| | | agctcggctcttcagcagcctcggtgagctgagcaccatctcagcgcagcgcagcccggggggcccggg |
| | | cggaggggcctcctactcggtgcggcccagcggccggtaccccgtggcgagacgagccccgagccca |
| | | gtgaaacccgcatcgctggagcgggtggaggggctggggggggcgtgggaggcgcggggcggccc |
| | | ttcggcctcacgcctcccaccatcctcaagtcgtccagcctctccatcccgcacgaacccaaggaagtgcg |
| | | cttcgtggtgcgaagtgtgagtgcgcgcagccgctccccctcaccatctccgctgccctcgccttctcccgg |
| | | ctctggccccagtgccggcccgcgtcggccatttcaacagaagcccctgcagctctggagcaagttcgatg |
| | | tgggcgactggctggagagcatccacttaggcgagcaccgagaccgcttcgaggaccatgagatcgaag |
| | | gcgcacacctgcctgcgctcaccaaggaagacttcgtggagctgggcgtcacacgcgttggccaccgcat |
| | | gaacatcgagcgtgcgctcaggcagctggatggcagctga |
| 4 | Human | atggacggccccggggccagcgccgtggtcgtgcgcgtcggcatcccggacctgcagcagacgaagtg |
| | miniShank3- | cctgcgcctggacccggccgcgcccgtgtgggccgccaagcagcgcgtgctctgcgccctcaaccacag |
| | V2 | cctccaggacgcgctcaactatgggcttttccagccgccctcccggggccgcgccggcaagttcctggatg |
| | | aggagcggctcctgcaggagtacccgcccaacctggacacgcccctgccctacctggagtttcgatacaa |
| | | gcggcgagtttatgcccagaacctcatcgatgataagcagtttgcaaagcttcacacaaaggcgaacctgaa |
| | | gaagttcatggactacgtccagctgcatagcacggacaaggtggcacgcctgttggacaaggggctggac |
| | | cccaacttccatgaccctgactcaggagagtgccccctgagcctcgcagcccagctggacaacgccacgg |
| | | acctgctaaaggtgctgaagaatggtggtgcccacctggacttccgcactcgcgatgggctcactgccgtg |
| | | cactgtgccacacgccagcggaatgcggcagcactgacgaccctgctggacctgggggcttcacctgact |
| | | acaaggacagccgcggcttgacacccctctaccacagcgccctggggggtggggatgccctctgctgtga |
| | | gctgcttctccacgaccacgctcagctggggatcaccgacgagaatggctggcaggagatccaccaggcc |
| | | tgccgctttgggcacgtgcagcatctggagcacctgctgttctatggggcagacatgggggcccagaacgc |
| | | ctcggggaacacagccctgcacatctgtgccctctacaaccaggagagctgtgctcgtgtcctgctcttccgt |
| | | ggagctaacagggatgtccgcaactacaacagccagacagccttccaggtggccatcatcgcagggaact |
| | | ttgagcttgcagaggttatcaagacccacaaagactcggatgttgtaccattcagggaaacccccagctatg |
| | | cgaagcggcggcgactggctggccccagtggcttggcatcccctcggcctctgcagcgctcagccagcg |
| | | atatcaacctgaagggggcaccgccgccccggggcccgaagcggaaactttacagcgccgtccccggc |
| | | cgcaagttcatcgccgtgaaggcgcacagcccgcagggtgaaggcgagatcccgctgcaccgcggcga |
| | | ggccgtgaaggtgctcagcattggggagggcggtttctgggagggaaccgtgaaaggccgcacgggctg |
| | | gttcccggccgactgcgtggaggaagtgcagatgaggcagcatgacacacggcctgaaacgcgggagg |
| | | accggacgaagcggctctttcggcactacacagtgggctcctacgacagcctcacctcacacagcgattat |
| | | gtcattgatgacaaagtggctgtcctgcagaaacgggaccacgagggctttggttttgtgctccggggagcc |
| | | aaagcagagacccccatcgaggagttcacgcccacgccagccttcccggcgctgcagtatctcgagtcgg |
| | | tggacgtggagggtgtggcctggagggccgggctgcgcacgggagacttcctcatcgaggtgaacggg |
| | | gtgaacgtggtgaaggtcggacacaagcaggtggtggctctgattcgccagggtggcaaccgcctcgtca |
| | | tgaaggttgtgtctgtgacaaggaagccagaagaggacggggctcggcgcagagccccaccgcccccc |
| | | aagagggcccccagcaccacactgaccctgcgctccaagtccatgacagctgagctcgaggaacttgcct |
| | | ccattcggagctcagaggaagagccagagctggtgtttgctgtgaacctgccacctgcccagctgtcgtcc |
| | | agcgatgaggagaccagggaggagctggcccgaattgggttggtgccaccccctgaagagtttgccaac |
| | | ggggtcctgctggccaccccactcgctggcccgggcccctcgcccaccacggtgcccagcccggcctca |
| | | gggaagcccagcagtgagccaccccctgcccctgagtctgcagccgactctggggggaggaggctgac |
| | | acacgcagctccagcgacccccacctggagaccacaagcaccatctccacggtgtccagcatgtccacctt |
| | | gagctcggagagcggggaactcactgacacccacacctccttcgctgacggacacacttttctactcgaga |
| | | agccaccagtgcctcccaagcccaagctcaagtccccgctggggaaggggccggtgaccttcagggacc |
| | | cgctgctgaagcagtcctcggacagcgagctcatggcccagcagcaccacgccgcctctgccgggctgg |
| | | cctctgccgccgggcctgcccgccctcgctacctcttccagagaaggtccaagctatggggggaccccgt |
| | | ggagagccgggggctccctgggcctgaagacgacaaaccaactgtgatcagtgagctcagctcccgcct |
| | | gcagcagctgaacaaggacacgcgttccctgggggaggaaccagttggtggcctgggcagcctgctgga |
| | | ccctgccaagaagtcgcccatcgcagcagctcggctcttcagcagcctcggtgagctgagctccatttcag |
| | | cgcagcgcagccccgggggcccgggggggggcctcgtactcggtgaggcccagtggccgctaccc |
| | | cgtggcgagacgcgccccgagcccggtgaagcccgcgtcgctggagcgggtggaggggctgggggc |
| | | gggcgcggggggcgcagggcggcccttcggcctcacgccccccaccatcctcaagtcgtccagcctctc |
| | | catcccgcacgagcccaaggaggtgcgcttcgtggtgcgcagcgtgagcgcgcgcagtcgctccccctc |
| | | gccgtcgccgctgccctcgcccgcgtccggccccggccccggcgcccccggcccacgccgacccttcc |
| | | agcagaagccgctgcagctctggagcaagttcgacgtgggcgactggctggagagcatccacctaggcg |
| | | agcaccgcgaccgcttcgaggaccatgagatagaaggcgcgcacctacccgcgcttaccaaggacgact |
| | | tcgtggagctgggcgtcacgcgcgtgggccaccgcatgaacatcgagcgcgcgctcaggcagctggac |
| | | ggcagctga |
| 7 | AAV- hSyn- | cctgcaggcagctgcgcgctcgctcgctcactgaggccgcccgggcaaagcccgggcgtcgggcgacc |
| | EGFP- | tttggtcgcccggcctcagtgagcgagcgagcgcgcagagagggagtggccaactccatcactaggggtt |
| | miniShank3 | cctgcggccgcacgcgtttaattaagtgtctagactgcagagggccctgcgtatgagtgcaagtgggttttag |
| | (5′ITR-hSyn- | gaccaggatgaggcgggggggggtgcctacctgacgaccgaccccgacccactggacaagcacccaa |
| | EGFP- | cccccattccccaaattgcgcatcccctatcagagagggggaggggaaacaggatgcggcgaggcgcgt |
| | miniShank3- | gcgcactgccagcttcagcaccgcggacagtgccttcgcccccgcctggcggcgcgcgccaccgccgc |
| | polyA- | ctcagcactgaaggcgcgctgacgtcactcgccggtcccccgcaaactccccttcccggccaccttggtcg |
| | WPRE- | cgtccgcgccgccgccggcccagccggaccgcaccacgcgaggcgcgagataggggggcacgggcg |
| | 3′ITR) | cgaccatctgcgctgcggcgccggcgactcagcgctgcctcagtctgcggtgggcagcggaggagtcgt |
| | | gtcgtgcctgagagcgcagtcgagaaaccggctagaggatccttcgaaaccggtgctagcagcgctgtta |
| | | acaccatggtgagcaagggcgaggagctgttcaccggggtggtgcccatcctggtcgagctggacggcg |
| | | acgtaaacggccacaagttcagcgtgtccggcgagggcgagggcgatgccacctacggcaagctgaccc |
| | | tgaagttcatctgcaccaccggcaagctgcccgtgccctggcccaccctcgtgaccaccctgacctacggc |
| | | gtgcagtgcttcagccgctaccccgaccacatgaagcagcacgacttcttcaagtccgccatgcccgaagg |
| | | ctacgtccaggagcgcaccatcttcttcaaggacgacggcaactacaagacccgcgccgaggtgaagttc |
| | | gagggcgacaccctggtgaaccgcatcgagctgaagggcatcgacttcaaggaggacggcaacatcctg |
| | | gggcacaagctggagtacaactacaacagccacaacgtctatatcatggccgacaagcagaagaacggc |
| | | atcaaggtgaacttcaagatccgccacaacatcgaggacggcagcgtgcagctcgccgaccactaccagc |
| | | agaacacccccatcggcgacggccccgtgctgctgcccgacaaccactacctgagcacccagtccgccct |
| | | gagcaaagaccccaacgagaagcgcgatcacatggtcctgctggagttcgtgaccgccgccgggatcact |
| | | ctcggcatggacgagctgtacaagggaagcggaggaattcacggcccgaagcggaaactttacagtgcc |
| | | gtccccggccgcaagttcatcgctgtgaaggcgcacagcccgcagggcgagggcgagatcccgctgca |
| | | ccgcggcgaggccgtgaaggtgctcagcattggggagggcggtttctgggagggaaccgtgaagggcc |
| | | gaacaggctggttcccagctgactgtgtggaagaagtgcagatgcgacagtatgacacccggcatgaaac |
| | | cagagaggaccggacgaagcgtctcttccgccactacactgtgggttcctatgacagcctcacttcacacag |
| | | cgattatgtcatcgatgataaggtggctatcctgcagaaaagggaccatgaggggtttggctttgttctccgg |
| | | ggagccaaagcagagacccccattgaggagtttacacccacacctgccttccctgcactccaataccttgag |
| | | tctgtagatgtggaaggtgtggcctggagggctggacttcgaactggggacttcctcattgaggtgaacgga |
| | | gtgaatgtcgtgaaggttggacacaagcaagtggtgggtctcatccgtcagggtggcaaccgcctggtcat |
| | | gaaggttgtgtctgtgaccaggaaacccgaggaggatggtgctcggcgcagagccccaccacccccaaa |
| | | gagggctcccagcaccacgctgaccctgcggtccaagtccatgacggctgagctcgaggaacttgcttcc |
| | | attcggagctcagaggaggagccagagctggtattcgctgtgaacctgccacctgctcagctgtcctccag |
| | | cgatgaggagaccagagaggagctggcccgcatagggctagtgccaccccctgaagagtttgccaatgg |
| | | gatcctgctgaccaccccgcccccagggccgggccccttgcccaccacggtacccagcccggcctcagg |
| | | gaagcccagcagcgagctgccccctgcccctgagtctgcagctgactctggagtagaggaggctgacact |
| | | cgaagctccagtgacccccacctggagaccacaagcaccatttccacagtgtccagcatgtccaccctgag |
| | | ctcggagagtggggaactcacggacacccacacctcctttgccgatggacacacttttctactcgagaagcc |
| | | accagtgcctcccaagcccaagctcaagtccccgctggggaaggggccggtgaccttcagggacccgct |
| | | gctgaagcaatcctcggacagtgagctcatggcccagcagcaccatgctgcctctactgggttggcttctgc |
| | | tgctgggcccgcccgccctcgctacctcttccagagaaggtccaagctgtggggggaccccgtggagagt |
| | | cgggggctccctgggcctgaagatgacaaaccaactgtgatcagtgagctcagctcccgtctgcagcagct |
| | | gaataaagacacacgctccttgggggaggaaccagttggtggcctgggcagcctgctggaccctgctaag |
| | | aagtcacccattgcagcagctcggctcttcagcagcctcggtgagctgagcaccatctcagcgcagcgcag |
| | | cccggggggcccgggcggaggggcctcctactcggtgcggcccagcggccggtaccccgtggcgaga |
| | | cgagccccgagcccagtgaaacccgcatcgctggagcgggtggaggggctgggggcgggcgtgggag |
| | | gcgcggggggcccttcggcctcacgcctcccaccatcctcaagtcgtccagcctctccatcccgcacgaa |
| | | cccaaggaagtgcgcttcgtggtgcgaagtgtgagtgcgcgcagccgctccccctcaccatctccgctgcc |
| | | ctcgccttctcccggctctggccccagtgccggcccgcgtcggccatttcaacagaagcccctgcagctct |
| | | ggagcaagttcgatgtgggcgactggctggagagcatccacttaggcgagcaccgagaccgcttcgagg |
| | | accatgagatcgaaggcgcacacctgcctgcgctcaccaaggaagacttcgtggagctgggcgtcacacg |
| | | cgttggccaccgcatgaacatcgagcgtgcgctcaggcagctggatggcagctgagcggccatcgtcgac |
| | | ggcgcgccaagcttatcgataatcaacctctggattacaaaatttgtgaaagattgactggtattcttaactatgt |
| | | tgctccttttacgctatgtggatacgctgctttaatgcctttgtatcatgctattgcttcccgtatggctttcattttct |
| | | cctccttgtataaatcctggttgctgtctctttatgaggagttgtggcccgttgtcaggcaacgtggcgtggtgt |
| | | gcactgtgtttgctgacgcaacccccactggttggggcattgccaccacctgtcagctcctttccgggactttc |
| | | gctttccccctccctattgccacggcggaactcatcgccgcctgccttgcccgctgctggacaggggctcgg |
| | | ctgttgggcactgacaattccgtggtgttgtcggggaaatcatcgtcctttccttggctgctcgcctatgttgcc |
| | | acctggattctgcgcgggacgtccttctgctacgtcccttcggccctcaatccagcggaccttccttcccgcg |
| | | gcctgctgccggctctgcggcctcttccgcgtcttcgccttcgccctcagacgagtcggatctccctttgggc |
| | | cgcctccccgcatcgataccgagcgctgctcgagagatctacgggtggcatccctgtgacccctccccagt |
| | | gcctctcctggccctggaagttgccactccagtgcccaccagccttgtcctaataaaattaagttgcatcatttt |
| | | gtctgactaggtgtccttctataatattatggggtggaggggggtggtatggagcaaggggcaagttgggaa |
| | | gacaacctgtagggcctgcggggtctattgggaaccaagctggagtgcagtggcacaatcttggctcactg |
| | | caatctccgcctcctgggttcaagcgattctcctgcctcagcctcccgagttgttgggattccaggcatgcatg |
| | | accaggctcagctaatttttgtttttttggtagagacggggtttcaccatattggccaggctggtctccaactcct |
| | | aatctcaggtgatctacccaccttggcctcccaaattgctgggattacaggcgtgaaccactgctcccttccct |
| | | gtccttctgattttgtaggtaaccacgtgcggaccgagcggccgcaggaacccctagtgatggagttggcca |
| | | ctccctctctgcgcgctcgctcgctcactgaggccgggcgaccaaaggtcgcccgacgcccgggctttgc |
| | | ccgggcggcctcagtgagcgagcgagcgcgcagctgcctgcaggggcgcctgatgcggtattttctcctt |
| | | acgcatctgtgcggtatttcacaccgcatacgtcaaagcaaccatagtacgcgccctgtagcggcgcattaa |
| | | gcgcggcgggtgtggtggttacgcgcagcgtgaccgctacacttgccagcgccctagcgcccgctcctttc |
| | | gctttcttcccttcctttctcgccacgttcgccggctttccccgtcaagctctaaatcgggggctccctttagggt |
| | | tccgatttagtgctttacggcacctcgaccccaaaaaacttgatttgggtgatggttcacgtagtgggccatcg |
| | | ccctgatagacggtttttcgccctttgacgttggagtccacgttctttaatagtggactcttgttccaaactggaa |
| | | caacactcaaccctatctcgggctattcttttgatttataagggattttgccgatttcggcctattggttaaaaaat |
| | | gagctgatttaacaaaaatttaacgcgaattttaacaaaatattaacgtttacaattttatggtgcactctcagtac |
| | | aatctgctctgatgccgcatagttaagccagccccgacacccgccaacacccgctgacgcgccctgacgg |
| | | gcttgtctgctcccggcatccgcttacagacaagctgtgaccgtctccgggagctgcatgtgtcagaggtttt |
| | | caccgtcatcaccgaaacgcgcgagacgaaagggcctcgtgatacgcctatttttataggttaatgtcatgat |
| | | aataatggtttcttagacgtcaggtggcacttttcggggaaatgtgcgcggaacccctatttgtttatttttctaaa |
| | | tacattcaaatatgtatccgctcatgagacaataaccctgataaatgcttcaataatattgaaaaaggaagagta |
| | | tgagtattcaacatttccgtgtcgcccttattcccttttttgcggcattttgccttcctgtttttgctcacccagaaac |
| | | gctggtgaaagtaaaagatgctgaagatcagttgggtgcacgagtgggttacatcgaactggatctcaacag |
| | | cggtaagatccttgagagttttcgccccgaagaacgttttccaatgatgagcacttttaaagttctgctatgtgg |
| | | cgcggtattatcccgtattgacgccgggcaagagcaactcggtcgccgcatacactattctcagaatgacttg |
| | | gttgagtactcaccagtcacagaaaagcatcttacggatggcatgacagtaagagaattatgcagtgctgcc |
| | | ataaccatgagtgataacactgcggccaacttacttctgacaacgatcggaggaccgaaggagctaaccgc |
| | | ttttttgcacaacatgggggatcatgtaactcgccttgatcgttgggaaccggagctgaatgaagccatacca |
| | | aacgacgagcgtgacaccacgatgcctgtagcaatggcaacaacgttgcgcaaactattaactggcgaact |
| | | acttactctagcttcccggcaacaattaatagactggatggaggcggataaagttgcaggaccacttctgcgc |
| | | tcggcccttccggctggctggtttattgctgataaatctggagccggtgagcgtgggtctcgcggtatcattgc |
| | | agcactggggccagatggtaagccctcccgtatcgtagttatctacacgacggggagtcaggcaactatgg |
| | | atgaacgaaatagacagatcgctgagataggtgcctcactgattaagcattggtaactgtcagaccaagttta |
| | | ctcatatatactttagattgatttaaaacttcatttttaatttaaaaggatctaggtgaagatcctttttgataatctca |
| | | tgaccaaaatcccttaacgtgagttttcgttccactgagcgtcagaccccgtagaaaagatcaaaggatcttct |
| | | tgagatcctttttttctgcgcgtaatctgctgcttgcaaacaaaaaaaccaccgctaccagcggtggtttgtttg |
| | | ccggatcaagagctaccaactctttttccgaaggtaactggcttcagcagagcgcagataccaaatactgtcc |
| | | ttctagtgtagccgtagttaggccaccacttcaagaactctgtagcaccgcctacatacctcgctctgctaatc |
| | | ctgttaccagtggctgctgccagtggcgataagtcgtgtcttaccgggttggactcaagacgatagttaccgg |
| | | ataaggcgcagcggtcgggctgaacggggggttcgtgcacacagcccagcttggagcgaacgacctaca |
| | | ccgaactgagatacctacagcgtgagctatgagaaagcgccacgcttcccgaagggagaaaggcggaca |
| | | ggtatccggtaagcggcagggtcggaacaggagagcgcacgagggagcttccagggggaaacgcctg |
| | | gtatctttatagtcctgtcgggtttcgccacctctgacttgagcgtcgatttttgtgatgctcgtcaggggggcg |
| | | gagcctatggaaaaacgccagcaacgcggcctttttacggttcctggccttttgctggccttttgctcacatgt |
| 8 | AAV- hSyn- | cctgcaggcagctgcgcgctcgctcgctcactgaggccgcccgggcaaagcccgggcgtcgggcgacc |
| | GFP (5′ITR- | tttggtcgcccggcctcagtgagcgagcgagcgcgcagagagggagtggccaactccatcactaggggtt |
| | hSyn-EGFP- | cctgcggccgcacgcgtttaattaagtgtctagactgcagagggccctgcgtatgagtgcaagtgggttttag |
| | polyA- | gaccaggatgaggcgggggggggtgcctacctgacgaccgaccccgacccactggacaagcacccaa |
| | WPRE- | cccccattccccaaattgcgcatcccctatcagagagggggaggggaaacaggatgcggcgaggcgcgt |
| | 3′ITR) | gcgcactgccagcttcagcaccgcggacagtgccttcgcccccgcctggcggcgcgcgccaccgccgc |
| | | ctcagcactgaaggcgcgctgacgtcactcgccggtcccccgcaaactccccttcccggccaccttggtcg |
| | | cgtccgcgccgccgccggcccagccggaccgcaccacgcgaggcgcgagataggggggcacgggcg |
| | | cgaccatctgcgctgcggcgccggcgactcagcgctgcctcagtctgcggtgggcagcggaggagtcgt |
| | | gtcgtgcctgagagcgcagtcgagaaaccggctagaggatccttcgaaaccggtgctagcagcgctgtta |
| | | acaccatggtgagcaagggcgaggagctgttcaccggggtggtgcccatcctggtcgagctggacggcg |
| | | acgtaaacggccacaagttcagcgtgtccggcgagggcgagggcgatgccacctacggcaagctgaccc |
| | | tgaagttcatctgcaccaccggcaagctgcccgtgccctggcccaccctcgtgaccaccctgacctacggc |
| | | gtgcagtgcttcagccgctaccccgaccacatgaagcagcacgacttcttcaagtccgccatgcccgaagg |
| | | ctacgtccaggagcgcaccatcttcttcaaggacgacggcaactacaagacccgcgccgaggtgaagttc |
| | | gagggcgacaccctggtgaaccgcatcgagctgaagggcatcgacttcaaggaggacggcaacatcctg |
| | | gggcacaagctggagtacaactacaacagccacaacgtctatatcatggccgacaagcagaagaacggc |
| | | atcaaggtgaacttcaagatccgccacaacatcgaggacggcagcgtgcagctcgccgaccactaccagc |
| | | agaacacccccatcggcgacggccccgtgctgctgcccgacaaccactacctgagcacccagtccgccct |
| | | gagcaaagaccccaacgagaagcgcgatcacatggtcctgctggagttcgtgaccgccgccgggatcact |
| | | ctcggcatggacgagctgtacaagtaaggcgcgcccctgcagggaattcgatatcaagcttatcgataatca |
| | | acctctggattacaaaatttgtgaaagattgactggtattcttaactatgttgctccttttacgctatgtggatacg |
| | | ctgctttaatgcctttgtatcatgctattgcttcccgtatggctttcattttctcctccttgtataaatcctggttgctgt |
| | | ctctttatgaggagttgtggcccgttgtcaggcaacgtggcgtggtgtgcactgtgtttgctgacgcaacccc |
| | | cactggttggggcattgccaccacctgtcagctcctttccgggactttcgctttccccctccctattgccacgg |
| | | cggaactcatcgccgcctgccttgcccgctgctggacaggggctcggctgttgggcactgacaattccgtg |
| | | gtgttgtcggggaaatcatcgtcctttccttggctgctcgcctatgttgccacctggattctgcgcgggacgtc |
| | | cttctgctacgtcccttcggccctcaatccagcggaccttccttcccgcggcctgctgccggctctgcggcct |
| | | cttccgcgtcttcgccttcgccctcagacgagtcggatctccctttgggccgcctccccgcatcgataccgag |
| | | cgctgctcgagagatctacgggtggcatccctgtgacccctccccagtgcctctcctggccctggaagttgc |
| | | cactccagtgcccaccagccttgtcctaataaaattaagttgcatcattttgtctgactaggtgtccttctataata |
| | | ttatggggtggaggggggtggtatggagcaaggggcaagttgggaagacaacctgtagggcctgcgggg |
| | | tctattgggaaccaagctggagtgcagtggcacaatcttggctcactgcaatctccgcctcctgggttcaagc |
| | | gattctcctgcctcagcctcccgagttgttgggattccaggcatgcatgaccaggctcagctaatttttgttttttt |
| | | ggtagagacggggtttcaccatattggccaggctggtctccaactcctaatctcaggtgatctacccaccttg |
| | | gcctcccaaattgctgggattacaggcgtgaaccactgctcccttccctgtccttctgattttgtaggtaaccac |
| | | gtgcggaccgagcggccgcaggaacccctagtgatggagttggccactccctctctgcgcgctcgctcgc |
| | | tcactgaggccgggcgaccaaaggtcgcccgacgcccgggctttgcccgggcggcctcagtgagcgag |
| | | cgagcgcgcagctgcctgcaggggcgcctgatgcggtattttctccttacgcatctgtgcggtatttcacacc |
| | | gcatacgtcaaagcaaccatagtacgcgccctgtagcggcgcattaagcgcggcgggtgtggtggttacg |
| | | cgcagcgtgaccgctacacttgccagcgccctagcgcccgctcctttcgctttcttcccttcctttctcgccac |
| | | gttcgccggctttccccgtcaagctctaaatcgggggctccctttagggttccgatttagtgctttacggcacct |
| | | cgaccccaaaaaacttgatttgggtgatggttcacgtagtgggccatcgccctgatagacggtttttcgccctt |
| | | tgacgttggagtccacgttctttaatagtggactcttgttccaaactggaacaacactcaaccctatctcgggct |
| | | attcttttgatttataagggattttgccgatttcggcctattggttaaaaaatgagctgatttaacaaaaatttaacg |
| | | cgaattttaacaaaatattaacgtttacaattttatggtgcactctcagtacaatctgctctgatgccgcatagtta |
| | | agccagccccgacacccgccaacacccgctgacgcgccctgacgggcttgtctgctcccggcatccgctt |
| | | acagacaagctgtgaccgtctccgggagctgcatgtgtcagaggttttcaccgtcatcaccgaaacgcgcg |
| | | agacgaaagggcctcgtgatacgcctatttttataggttaatgtcatgataataatggtttcttagacgtcaggt |
| | | ggcacttttcggggaaatgtgcgcggaacccctatttgtttatttttctaaatacattcaaatatgtatccgctcat |
| | | gagacaataaccctgataaatgcttcaataatattgaaaaaggaagagtatgagtattcaacatttccgtgtcg |
| | | cccttattcccttttttgcggcattttgccttcctgtttttgctcacccagaaacgctggtgaaagtaaaagatgct |
| | | gaagatcagttgggtgcacgagtgggttacatcgaactggatctcaacagcggtaagatccttgagagttttc |
| | | gccccgaagaacgttttccaatgatgagcacttttaaagttctgctatgtggcgcggtattatcccgtattgacg |
| | | ccgggcaagagcaactcggtcgccgcatacactattctcagaatgacttggttgagtactcaccagtcacag |
| | | aaaagcatcttacggatggcatgacagtaagagaattatgcagtgctgccataaccatgagtgataacactg |
| | | cggccaacttacttctgacaacgatcggaggaccgaaggagctaaccgcttttttgcacaacatgggggatc |
| | | atgtaactcgccttgatcgttgggaaccggagctgaatgaagccataccaaacgacgagcgtgacaccacg |
| | | atgcctgtagcaatggcaacaacgttgcgcaaactattaactggcgaactacttactctagcttcccggcaac |
| | | aattaatagactggatggaggcggataaagttgcaggaccacttctgcgctcggcccttccggctggctggt |
| | | ttattgctgataaatctggagccggtgagcgtgggtctcgcggtatcattgcagcactggggccagatggta |
| | | agccctcccgtatcgtagttatctacacgacggggagtcaggcaactatggatgaacgaaatagacagatc |
| | | gctgagataggtgcctcactgattaagcattggtaactgtcagaccaagtttactcatatatactttagattgattt |
| | | aaaacttcatttttaatttaaaaggatctaggtgaagatcctttttgataatctcatgaccaaaatcccttaacgtg |
| | | agttttcgttccactgagcgtcagaccccgtagaaaagatcaaaggatcttcttgagatcctttttttctgcgcgt |
| | | aatctgctgcttgcaaacaaaaaaaccaccgctaccagcggtggtttgtttgccggatcaagagctaccaact |
| | | ctttttccgaaggtaactggcttcagcagagcgcagataccaaatactgtccttctagtgtagccgtagttagg |
| | | ccaccacttcaagaactctgtagcaccgcctacatacctcgctctgctaatcctgttaccagtggctgctgcca |
| | | gtggcgataagtcgtgtcttaccgggttggactcaagacgatagttaccggataaggcgcagcggtcgggc |
| | | tgaacggggggttcgtgcacacagcccagcttggagcgaacgacctacaccgaactgagatacctacagc |
| | | gtgagctatgagaaagcgccacgcttcccgaagggagaaaggcggacaggtatccggtaagcggcagg |
| | | gtcggaacaggagagcgcacgagggagcttccagggggaaacgcctggtatctttatagtcctgtcgggtt |
| | | tcgccacctctgacttgagcgtcgatttttgtgatgctcgtcaggggggcggagcctatggaaaaacgccag |
| | | caacgcggcctttttacggttcctggccttttgctggccttttgctcacatgt |
| 9 | AAV- hSyn- | tcctgcaggcagctgcgcgctcgctcgctcactgaggccgcccgggcaaagcccgggcgtcgggcgac |
| | nCre-EGFP | ctttggtcgcccggcctcagtgagcgagcgagcgcgcagagagggagtggccaactccatcactagggg |
| | (5′ITR-hSyn- | ttcctgcggccgcacgcgtttaattaagtgtctagactgcagagggccctgcgtatgagtgcaagtgggtttt |
| | EGFP-Cre- | aggaccaggatgaggcgggggggggtgcctacctgacgaccgaccccgacccactggacaagcaccc |
| | polyA- | aacccccattccccaaattgcgcatcccctatcagagagggggaggggaaacaggatgcggcgaggcgc |
| | WPRE- | gtgcgcactgccagcttcagcaccgcggacagtgccttcgcccccgcctggggcgcgcgccaccgccg |
| | 3′ITR) | cctcagcactgaaggcgcgctgacgtcactcgccggtcccccgcaaactccccttcccggccaccttggtc |
| | | gcgtccgcgccgccgccggcccagccggaccgcaccacgcgaggcgcgagataggggggcacgggc |
| | | gcgaccatctgcgctgcggcgccggcgactcagcgctgcctcagtctgcggtgggcagcggaggagtcg |
| | | tgtcgtgcctgagagcgcagtcgagaaaccggctagaggatccttcgaaaccggtgctagcaccatgccta |
| | | agaagaagagaaaggtatgcaggtcacaccaaaatttgcctgcattaccggtcgatgcaacgagtgatgag |
| | | gttcgcaagaacctgatggacatgttcagggatcgccaggcgttttctgagcatacctggaaaatgcttctgt |
| | | ccgtttgccggtcgtgggcggcatggtgcaagttgaataaccggaaatggtttcccgcagaacctgaagat |
| | | gttcgcgattatcttctatatcttcaggcgcgcggtctggcagtaaaaactatccagcaacatttgggccagct |
| | | aaacatgcttcatcgtcggtccgggctgccacgaccaagtgacagcaatgctgtttcactggttatgcggcg |
| | | gatccgaaaagaaaacgttgatgccggtgaacgtgcaaaacaggctctagcgttcgaacgcactgatttcg |
| | | accaggttcgttcactcatggaaaatagcgatcgctgccaggatatacgtaatctggcatttctggggattgct |
| | | tataacaccctgttacgtatagccgaaattgccaggatcagggttaaagatatctcacgtactgacggtggga |
| | | gaatgttaatccatattggcagaacgaaaacgctggttagcaccgcaggtgtagagaaggcacttagcctg |
| | | ggggtaactaaactggtcgagcgatggatttccgtctctggtgtagctgatgatccgaataactacctgttttg |
| | | ccgggtcagaaaaaatggtgttgccgcgccatctgccaccagccagctatcaactcgcgccctggaaggg |
| | | atttttgaagcaactcatcgattgatttacggcgctaaggatgactctggtcagagatacctggcctggtctgg |
| | | acacagtgcccgtgtcggagccgcgcgagatatggcccgcgctggagtttcaataccggagatcatgcaa |
| | | gctggtggctggaccaatgtaaatattgtcatgaactatatccgtaacctggatagtgaaacaggggcaatgg |
| | | tgcgcctgctggaagatggcgatgttaacgtgagcaagggcgaggagctgttcaccggggtggtgcccat |
| | | cctggtcgagctggacggcgacgtaaacggccacaagttcagcgtgtccggcgagggcgagggcgatg |
| | | ccacctacggcaagctgaccctgaagttcatctgcaccaccggcaagctgcccgtgccctggcccaccctc |
| | | gtgaccaccctgacctacggcgtgcagtgcttcagccgctaccccgaccacatgaagcagcacgacttctt |
| | | caagtccgccatgcccgaaggctacgtccaggagcgcaccatcttcttcaaggacgacggcaactacaag |
| | | acccgcgccgaggtgaagttcgagggcgacaccctggtgaaccgcatcgagctgaagggcatcgacttc |
| | | aaggaggacggcaacatcctggggcacaagctggagtacaactacaacagccacaacgtctatatcatgg |
| | | ccgacaagcagaagaacggcatcaaggtgaacttcaagatccgccacaacatcgaggacggcagcgtgc |
| | | agctcgccgaccactaccagcagaacacccccatcggcgacggccccgtgctgctgcccgacaaccact |
| | | acctgagcacccagtccgccctgagcaaagaccccaacgagaagcgcgatcacatggtcctgctggagtt |
| | | cgtgaccgccgccgggatcactctcggcatggacgagctgtacaagtaagtcgacggcgcgcccctgca |
| | | gggaattcgatatcaagcttatcgataatcaacctctggattacaaaatttgtgaaagattgactggtattcttaa |
| | | ctatgttgctccttttacgctatgtggatacgctgctttaatgcctttgtatcatgctattgcttcccgtatggctttc |
| | | attttctcctccttgtataaatcctggttgctgtctctttatgaggagttgtggcccgttgtcaggcaacgtggcgt |
| | | ggtgtgcactgtgtttgctgacgcaacccccactggttggggcattgccaccacctgtcagctcctttccggg |
| | | actttcgctttccccctccctattgccacggcggaactcatcgccgcctgccttgcccgctgctggacagggg |
| | | ctcggctgttgggcactgacaattccgtggtgttgtcggggaaatcatcgtcctttccttggctgctcgcctatg |
| | | ttgccacctggattctgcgcgggacgtccttctgctacgtcccttcggccctcaatccagcggaccttccttcc |
| | | cgcggcctgctgccggctctgcggcctcttccgcgtcttcgccttcgccctcagacgagtcggatctcccttt |
| | | gggccgcctccccgcatcgataccgagcgctgctcgagagatctacgggtggcatccctgtgacccctcc |
| | | ccagtgcctctcctggccctggaagttgccactccagtgcccaccagccttgtcctaataaaattaagttgcat |
| | | cattttgtctgactaggtgtccttctataatattatggggtggaggggggtggtatggagcaaggggcaagttg |
| | | ggaagacaacctgtagggcctgcggggtctattgggaaccaagctggagtgcagtggcacaatcttggctc |
| | | actgcaatctccgcctcctgggttcaagcgattctcctgcctcagcctcccgagttgttgggattccaggcatg |
| | | catgaccaggctcagctaatttttgtttttttggtagagacggggtttcaccatattggccaggctggtctccaa |
| | | ctcctaatctcaggtgatctacccaccttggcctcccaaattgctgggattacaggcgtgaaccactgctccct |
| | | tccctgtccttctgattttgtaggtaaccacgtgcggaccgagcggccgcaggaacccctagtgatggagtt |
| | | ggccactccctctctgcgcgctcgctcgctcactgaggccgggcgaccaaaggtcgcccgacgcccggg |
| | | ctttgcccgggcggcctcagtgagcgagcgagcgcgcagctgcctgcaggggcgcctgatgcggtatttt |
| | | ctccttacgcatctgtgcggtatttcacaccgcatacgtcaaagcaaccatagtacgcgccctgtagcggcg |
| | | cattaagcgcggcgggtgtggtggttacgcgcagcgtgaccgctacacttgccagcgccctagcgcccgc |
| | | tcctttcgctttcttcccttcctttctcgccacgttcgccggctttccccgtcaagctctaaatcgggggctccctt |
| | | tagggttccgatttagtgctttacggcacctcgaccccaaaaaacttgatttgggtgatggttcacgtagtggg |
| | | ccatcgccctgatagacggtttttcgccctttgacgttggagtccacgttctttaatagtggactcttgttccaaa |
| | | ctggaacaacactcaaccctatctcgggctattcttttgatttataagggattttgccgatttcggcctattggtta |
| | | aaaaatgagctgatttaacaaaaatttaacgcgaattttaacaaaatattaacgtttacaattttatggtgcactct |
| | | cagtacaatctgctctgatgccgcatagttaagccagccccgacacccgccaacacccgctgacgcgccct |
| | | gacgggcttgtctgctcccggcatccgcttacagacaagctgtgaccgtctccgggagctgcatgtgtcaga |
| | | ggttttcaccgtcatcaccgaaacgcgcgagacgaaagggcctcgtgatacgcctatttttataggttaatgtc |
| | | atgataataatggtttcttagacgtcaggtggcacttttcggggaaatgtgcgcggaacccctatttgtttattttt |
| | | ctaaatacattcaaatatgtatccgctcatgagacaataaccctgataaatgcttcaataatattgaaaaaggaa |
| | | gagtatgagtattcaacatttccgtgtcgcccttattcccttttttgcggcattttgccttcctgtttttgctcaccca |
| | | gaaacgctggtgaaagtaaaagatgctgaagatcagttgggtgcacgagtgggttacatcgaactggatctc |
| | | aacagcggtaagatccttgagagttttcgccccgaagaacgttttccaatgatgagcacttttaaagttctgcta |
| | | tgtggcgcggtattatcccgtattgacgccgggcaagagcaactcggtcgccgcatacactattctcagaat |
| | | gacttggttgagtactcaccagtcacagaaaagcatcttacggatggcatgacagtaagagaattatgcagt |
| | | gctgccataaccatgagtgataacactgcggccaacttacttctgacaacgatcggaggaccgaaggagct |
| | | aaccgcttttttgcacaacatgggggatcatgtaactcgccttgatcgttgggaaccggagctgaatgaagcc |
| | | ataccaaacgacgagcgtgacaccacgatgcctgtagcaatggcaacaacgttgcgcaaactattaactgg |
| | | cgaactacttactctagcttcccggcaacaattaatagactggatggaggcggataaagttgcaggaccactt |
| | | ctgcgctcggcccttccggctggctggtttattgctgataaatctggagccggtgagcgtgggtctcgcggta |
| | | tcattgcagcactggggccagatggtaagccctcccgtatcgtagttatctacacgacggggagtcaggca |
| | | actatggatgaacgaaatagacagatcgctgagataggtgcctcactgattaagcattggtaactgtcagacc |
| | | aagtttactcatatatactttagattgatttaaaacttcatttttaatttaaaaggatctaggtgaagatcctttttgat |
| | | aatctcatgaccaaaatcccttaacgtgagttttcgttccactgagcgtcagaccccgtagaaaagatcaaag |
| | | gatcttcttgagatcctttttttctgcgcgtaatctgctgcttgcaaacaaaaaaaccaccgctaccagcggtgg |
| | | tttgtttgccggatcaagagctaccaactctttttccgaaggtaactggcttcagcagagcgcagataccaaat |
| | | actgtccttctagtgtagccgtagttaggccaccacttcaagaactctgtagcaccgcctacatacctcgctct |
| | | gctaatcctgttaccagtggctgctgccagtggcgataagtcgtgtcttaccgggttggactcaagacgatag |
| | | ttaccggataaggcgcagcggtcgggctgaacggggggttcgtgcacacagcccagcttggagcgaacg |
| | | acctacaccgaactgagatacctacagcgtgagctatgagaaagcgccacgcttcccgaagggagaaag |
| | | gcggacaggtatccggtaagcggcagggtcggaacaggagagcgcacgagggagcttccagggggaa |
| | | acgcctggtatctttatagtcctgtcgggtttcgccacctctgacttgagcgtcgatttttgtgatgctcgtcagg |
| | | ggggcggagcctatggaaaaacgccagcaacgcggcctttttacggttcctggccttttgctggccttttgct |
| | | cacatg |
| 10 | AAV- | cctgcaggcagctgcgcgctcgctcgctcactgaggccgcccgggcaaagcccgggcgtcgggcgacc |
| | PHP.eBhSyn- | tttggtcgcccggcctcagtgagcgagcgagcgcgcagagagggagtggccaactccatcactaggggtt |
| | Delta-nCre- | cctgcggccgcacgcgtttaattaagtgtctagactgcagagggccctgcgtatgagtgcaagtgggttttag |
| | EGFP (5′ITR- | gaccaggatgaggcgggggggggtgcctacctgacgaccgaccccgacccactggacaagcacccaa |
| | hSyn-EGFP- | cccccattccccaaattgcgcatcccctatcagagagggggaggggaaacaggatgcggcgaggcgcgt |
| | Delta-Cre- | gcgcactgccagcttcagcaccgcggacagtgccttcgcccccgcctggcggcgcgcgccaccgccgc |
| | polyA- | ctcagcactgaaggcgcgctgacgtcactcgccggtcccccgcaaactccccttcccggccaccttggtcg |
| | WPRE- | cgtccgcgccgccgccggcccagccggaccgcaccacgcgaggcgcgagataggggggcacgggcg |
| | 3′ITR) | cgaccatctgcgctgcggcgccggcgactcagcgctgcctcagtctgcggtgggcagcggaggagtcgt |
| | | gtcgtgcctgagagcgcagtcgagaaaccggctagaggatccttcgaaaccggtgctagcaccatgccta |
| | | agaagaagagaaaggtatgcaggtcacaccaaaatttgcctgcattaccggtcgatgcaacgagtgatgag |
| | | gttcgcaagaacctgatggacatgttcagggatcgccaggcgttttctgagcatacctggaaaatgcttctgt |
| | | ccgtttgccggtcgtgggcggcatggtgcaagttgaataaccggaaatggtttcccgcagaacctgaagat |
| | | gttcgcgattatcttctatatcttcaggcgcgcggtctggcagtaaaaactatccagcaacatttgggccagct |
| | | aaacatgcttcatcgtcggtccgggctgccacgaccaagtgacagcaatgctgtttcactggttatgcggcg |
| | | gatccgagttaacgtgagcaagggcgaggagctgttcaccggggtggtgcccatcctggtcgagctggac |
| | | ggcgacgtaaacggccacaagttcagcgtgtccggcgagggcgagggcgatgccacctacggcaagct |
| | | gaccctgaagttcatctgcaccaccggcaagctgcccgtgccctggcccaccctcgtgaccaccctgacct |
| | | acggcgtgcagtgcttcagccgctaccccgaccacatgaagcagcacgacttcttcaagtccgccatgccc |
| | | gaaggctacgtccaggagcgcaccatcttcttcaaggacgacggcaactacaagacccgcgccgaggtg |
| | | aagttcgagggcgacaccctggtgaaccgcatcgagctgaagggcatcgacttcaaggaggacggcaac |
| | | atcctggggcacaagctggagtacaactacaacagccacaacgtctatatcatggccgacaagcagaaga |
| | | acggcatcaaggtgaacttcaagatccgccacaacatcgaggacggcagcgtgcagctcgccgaccacta |
| | | ccagcagaacacccccatcggcgacggccccgtgctgctgcccgacaaccactacctgagcacccagtc |
| | | cgccctgagcaaagaccccaacgagaagcgcgatcacatggtcctgctggagttcgtgaccgccgccgg |
| | | gatcactctcggcatggacgagctgtacaagtaagtcgacggcgcgcccctgcagggaattcgatatcaag |
| | | cttatcgataatcaacctctggattacaaaatttgtgaaagattgactggtattcttaactatgttgctccttttacg |
| | | ctatgtggatacgctgctttaatgcctttgtatcatgctattgcttcccgtatggctttcattttctcctccttgtataa |
| | | atcctggttgctgtctctttatgaggagttgtggcccgttgtcaggcaacgtggcgtggtgtgcactgtgtttgc |
| | | tgacgcaacccccactggttggggcattgccaccacctgtcagctcctttccgggactttcgctttccccctcc |
| | | ctattgccacggcggaactcatcgccgcctgccttgcccgctgctggacaggggctcggctgttgggcact |
| | | gacaattccgtggtgttgtcggggaaatcatcgtcctttccttggctgctcgcctatgttgccacctggattctg |
| | | cgcgggacgtccttctgctacgtcccttcggccctcaatccagcggaccttccttcccgcggcctgctgccg |
| | | gctctgcggcctcttccgcgtcttcgccttcgccctcagacgagtcggatctccctttgggccgcctccccgc |
| | | atcgataccgagcgctgctcgagagatctacgggtggcatccctgtgacccctccccagtgcctctcctggc |
| | | cctggaagttgccactccagtgcccaccagccttgtcctaataaaattaagttgcatcattttgtctgactaggt |
| | | gtccttctataatattatggggtggaggggggtggtatggagcaaggggcaagttgggaagacaacctgta |
| | | gggcctgcggggtctattgggaaccaagctggagtgcagtggcacaatcttggctcactgcaatctccgcct |
| | | cctgggttcaagcgattctcctgcctcagcctcccgagttgttgggattccaggcatgcatgaccaggctcag |
| | | ctaatttttgtttttttggtagagacggggtttcaccatattggccaggctggtctccaactcctaatctcaggtga |
| | | tctacccaccttggcctcccaaattgctgggattacaggcgtgaaccactgctcccttccctgtccttctgatttt |
| | | gtaggtaaccacgtgcggaccgagcggccgcaggaacccctagtgatggagttggccactccctctctgc |
| | | gcgctcgctcgctcactgaggccgggcgaccaaaggtcgcccgacgcccgggctttgcccgggcggcct |
| | | cagtgagcgagcgagcgcgcagctgcctgcaggggcgcctgatgcggtattttctccttacgcatctgtgc |
| | | ggtatttcacaccgcatacgtcaaagcaaccatagtacgcgccctgtagcggcgcattaagcgcggcgggt |
| | | gtggtggttacgcgcagcgtgaccgctacacttgccagcgccctagcgcccgctcctttcgctttcttcccttc |
| | | ctttctcgccacgttcgccggctttccccgtcaagctctaaatcgggggctccctttagggttccgatttagtgc |
| | | tttacggcacctcgaccccaaaaaacttgatttgggtgatggttcacgtagtgggccatcgccctgatagacg |
| | | gtttttcgccctttgacgttggagtccacgttctttaatagtggactcttgttccaaactggaacaacactcaacc |
| | | ctatctcgggctattcttttgatttataagggattttgccgatttcggcctattggttaaaaaatgagctgatttaac |
| | | aaaaatttaacgcgaattttaacaaaatattaacgtttacaattttatggtgcactctcagtacaatctgctctgat |
| | | gccgcatagttaagccagccccgacacccgccaacacccgctgacgcgccctgacgggcttgtctgctcc |
| | | cggcatccgcttacagacaagctgtgaccgtctccgggagctgcatgtgtcagaggttttcaccgtcatcac |
| | | cgaaacgcgcgagacgaaagggcctcgtgatacgcctatttttataggttaatgtcatgataataatggtttctt |
| | | agacgtcaggtggcacttttcggggaaatgtgcgcggaacccctatttgtttatttttctaaatacattcaaatat |
| | | gtatccgctcatgagacaataaccctgataaatgcttcaataatattgaaaaaggaagagtatgagtattcaac |
| | | atttccgtgtcgcccttattcccttttttgcggcattttgccttcctgtttttgctcacccagaaacgctggtgaaag |
| | | taaaagatgctgaagatcagttgggtgcacgagtgggttacatcgaactggatctcaacagcggtaagatcc |
| | | ttgagagttttcgccccgaagaacgttttccaatgatgagcacttttaaagttctgctatgtggcgcggtattatc |
| | | ccgtattgacgccgggcaagagcaactcggtcgccgcatacactattctcagaatgacttggttgagtactca |
| | | ccagtcacagaaaagcatcttacggatggcatgacagtaagagaattatgcagtgctgccataaccatgagt |
| | | gataacactgcggccaacttacttctgacaacgatcggaggaccgaaggagctaaccgcttttttgcacaac |
| | | atgggggatcatgtaactcgccttgatcgttgggaaccggagctgaatgaagccataccaaacgacgagcg |
| | | tgacaccacgatgcctgtagcaatggcaacaacgttgcgcaaactattaactggcgaactacttactctagct |
| | | tcccggcaacaattaatagactggatggaggcggataaagttgcaggaccacttctgcgctcggcccttccg |
| | | gctggctggtttattgctgataaatctggagccggtgagcgtgggtctcgcggtatcattgcagcactggggc |
| | | cagatggtaagccccccgtatcgtagttatctacacgacggggagtcaggcaactatggatgaacgaaata |
| | | gacagatcgctgagataggtgcctcactgattaagcattggtaactgtcagaccaagtttactcatatatacttt |
| | | agattgatttaaaacttcatttttaatttaaaaggatctaggtgaagatcctttttgataatctcatgaccaaaatcc |
| | | cttaacgtgagttttcgttccactgagcgtcagaccccgtagaaaagatcaaaggatcttcttgagatcctttttt |
| | | tctgcgcgtaatctgctgcttgcaaacaaaaaaaccaccgctaccagcggtggtttgtttgccggatcaagag |
| | | ctaccaactctttttccgaaggtaactggcttcagcagagcgcagataccaaatactgtccttctagtgtagcc |
| | | gtagttaggccaccacttcaagaactctgtagcaccgcctacatacctcgctctgctaatcctgttaccagtgg |
| | | ctgctgccagtggcgataagtcgtgtcttaccgggttggactcaagacgatagttaccggataaggcgcagc |
| | | ggtcgggctgaacggggggttcgtgcacacagcccagcttggagcgaacgacctacaccgaactgagat |
| | | acctacagcgtgagctatgagaaagcgccacgcttcccgaagggagaaaggcggacaggtatccggtaa |
| | | gcggcagggtcggaacaggagagcgcacgagggagcttccagggggaaacgcctggtatctttatagtc |
| | | ctgtcgggtttcgccacctctgacttgagcgtcgatttttgtgatgctcgtcaggggggcggagcctatggaa |
| | | aaacgccagcaacgcggcctttttacggttcctggccttttgctggccttttgctcacatgt |
| 11 | Protein | MVSKGEELFTGVVPILVELDGDVNGHKFSVSGEGEGDATYGKLTLK |
| | sequence | FICTTGKLPVPWPTLVTTLTYGVQCFSRYPDHMKQHDFFKSAMPEG |
| | encoded by | YVQERTIFFKDDGNYKTRAEVKFEGDTLVNRIELKGIDFKEDGNILG |
| | the transgene | HKLEYNYNSHNVYIMADKQKNGIKVNFKIRHNIEDGSVQLADHYQ |
| | of SEQ ID | QNTPIGDGPVLLPDNHYLSTQSALSKDPNEKRDHMVLLEFVTAAGIT |
| | NO: 7 (AAV- | LGMDELYKGSGGIHGPKRKLYSAVPGRKFIAVKAHSPQGEGEIPLH |
| | hSyn-EGFP- | RGEAVKVLSIGEGGFWEGTVKGRTGWFPADCVEEVQMRQYDTRH |
| | miniShank3) | ETREDRTKRLFRHYTVGSYDSLTSHSDYVIDDKVAILQKRDHEGFGF |
| | | VLRGAKAETPIEEFTPTPAFPALQYLESVDVEGVAWRAGLRTGDFLI |
| | | EVNGVNVVKVGHKQVVGLIRQGGNRLVMKVVSVTRKPEEDGARR |
| | | RAPPPPKRAPSTTLTLRSKSMTAELEELASIRSSEEEPELVFAVNLPPA |
| | | QLSSSDEETREELARIGLVPPPEEFANGILLTTPPPGPGPLPTTVPSPAS |
| | | GKPSSELPPAPESAADSGVEEADTRSSSDPHLETTSTISTVSSMSTLSS |
| | | ESGELTDTHTSFADGHTFLLEKPPVPPKPKLKSPLGKGPVTFRDPLL |
| | | KQSSDSELMAQQHHAASTGLASAAGPARPRYLFQRRSKLWGDPVE |
| | | SRGLPGPEDDKPTVISELSSRLQQLNKDTRSLGEEPVGGLGSLLDPA |
| | | KKSPIAAARLFSSLGELSTISAQRSPGGPGGGASYSVRPSGRYPVARR |
| | | APSPVKPASLERVEGLGAGVGGAGRPFGLTPPTILKSSSLSIPHEPKE |
| | | VRFVVRSVSARSRSPSPSPLPSPSPGSGPSAGPRRPFQQKPLQLWSKF |
| | | DVGDWLESIHLGEHRDRFEDHEIEGAHLPALTKEDFVELGVTRVGH |
| | | RMNIERALRQLDGS* |
| 12 | Protein | MVSKGEELFTGVVPILVELDGDVNGHKFSVSGEGEGDATYGKLTLK |
| | sequence | FICTTGKLPVPWPTLVTTLTYGVQCFSRYPDHMKQHDFFKSAMPEG |
| | encoded by | YVQERTIFFKDDGNYKTRAEVKFEGDTLVNRIELKGIDFKEDGNILG |
| | the transgene | HKLEYNYNSHNVYIMADKQKNGIKVNFKIRHNIEDGSVQLADHYQ |
| | of SEQ ID | QNTPIGDGPVLLPDNHYLSTQSALSKDPNEKRDHMVLLEFVTAAGIT |
| | NO: 8 (AAV- | LGMDELYK* |
| | hSyn-GFP) | |
| 13 | Protein | MPKKKRKVCRSHQNLPALPVDATSDEVRKNLMDMFRDRQAFSEHT |
| | sequence | WKMLLSVCRSWAAWCKLNNRKWFPAEPEDVRDYLLYLQARGLA |
| | encoded by | VKTIQQHLGQLNMLHRRSGLPRPSDSNAVSLVMRRIRKENVDAGER |
| | the transgene | AKQALAFERTDFDQVRSLMENSDRCQDIRNLAFLGIAYNTLLRIAEI |
| | of SEQ ID | ARIRVKDISRTDGGRMLIHIGRTKTLVSTAGVEKALSLGVTKLVERW |
| | NO: 9 (AAV- | ISVSGVADDPNNYLFCRVRKNGVAAPSATSQLSTRALEGIFEATHRL |
| | hSyn-nCre- | IYGAKDDSGQRYLAWSGHSARVGAARDMARAGVSIPEIMQAGGW |
| | EGFP) | TNVNIVMNYIRNLDSETGAMVRLLEDGDVNVSKGEELFTGVVPILV |
| | | ELDGDVNGHKFSVSGEGEGDATYGKLTLKFICTTGKLPVPWPTLVT |
| | | TLTYGVQCFSRYPDHMKQHDFFKSAMPEGYVQERTIFFKDDGNYK |
| | | TRAEVKFEGDTLVNRIELKGIDFKEDGNILGHKLEYNYNSHNVYIM |
| | | ADKQKNGIKVNFKIRHNIEDGSVQLADHYQQNTPIGDGPVLLPDNH |
| | | YLSTQSALSKDPNEKRDHMVLLEFVTAAGITLGMDELYK* |
| 14 | Protein | MPKKKRKVCRSHQNLPALPVDATSDEVRKNLMDMFRDRQAFSEHT |
| | sequence | WKMLLSVCRSWAAWCKLNNRKWFPAEPEDVRDYLLYLQARGLA |
| | encoded by | VKTIQQHLGQLNMLHRRSGLPRPSDSNAVSLVMRRIRVNVSKGEEL |
| | the transgene | FTGVVPILVELDGDVNGHKFSVSGEGEGDATYGKLTLKFICTTGKLP |
| | of SEQ ID | VPWPTLVTTLTYGVQCFSRYPDHMKQHDFFKSAMPEGYVQERTIFF |
| | NO: 10 | KDDGNYKTRAEVKFEGDTLVNRIELKGIDFKEDGNILGHKLEYNYN |
| | (AAV- | SHNVYIMADKQKNGIKVNFKIRHNIEDGSVQLADHYQQNTPIGDGP |
| | PHP.eBhSyn- | VLLPDNHYLSTQSALSKDPNEKRDHMVLLEFVTAAGITLGMDELYK |
| | Delta-nCre- | * |
| | EGFP) | |
| 17 | Mouse | MGPKRKLYSAVPGRKFIAVKAHSPQGEGEIPLHRGEAVKVLSIGEG |
| | miniShank3- | GFWEGTVKGRTGWFPADCVEEVQMRQYDTRHETREDRTKRLFRH |
| | V1 protein | YTVGSYDSLTSHSDYVIDDKVAILQKRDHEGFGFVLRGAKAETPIEE |
| | sequence | FTPTPAFPALQYLESVDVEGVAWRAGLRTGDFLIEVNGVNVVKVG |
| | encoded by | HKQVVGLIRQGGNRLVMKVVSVTRKPEEDGARRRAPPPPKRAPSTT |
| | SEQ ID NO: | LTLRSKSMTAELEELASIRSSEEEPELVFAVNLPPAQLSSSDEETREEL |
| | 1 | ARIGLVPPPEEFANGILLTTPPPGPGPLPTTVPSPASGKPSSELPPAPES |
| | | AADSGVEEADTRSSSDPHLETTSTISTVSSMSTLSSESGELTDTHTSF |
| | | ADGHTFLLEKPPVPPKPKLKSPLGKGPVTFRDPLLKQSSDSELMAQQ |
| | | HHAASTGLASAAGPARPRYLFQRRSKLWGDPVESRGLPGPEDDKPT |
| | | VISELSSRLQQLNKDTRSLGEEPVGGLGSLLDPAKKSPIAAARLFSSL |
| | | GELSTISAQRSPGGPGGGASYSVRPSGRYPVARRAPSPVKPASLERV |
| | | EGLGAGVGGAGRPFGLTPPTILKSSSLSIPHEPKEVRFVVRSVSARSR |
| | | SPSPSPLPSPSPGSGPSAGPRRPFQQKPLQLWSKFDVGDWLESIHLGE |
| | | HRDRFEDHEIEGAHLPALTKEDFVELGVTRVGHRMNIERALRQLDG |
| | | S* |
| 18 | Human | MGPKRKLYSAVPGRKFIAVKAHSPQGEGEIPLHRGEAVKVLSIGEG |
| | miniShank3- | GFWEGTVKGRTGWFPADCVEEVQMRQHDTRPETREDRTKRLFRHY |
| | V1 protein | TVGSYDSLTSHSDYVIDDKVAVLQKRDHEGFGFVLRGAKAETPIEEF |
| | sequence | TPTPAFPALQYLESVDVEGVAWRAGLRTGDFLIEVNGVNVVKVGH |
| | encoded by | KQVVALIRQGGNRLVMKVVSVTRKPEEDGARRRAPPPPKRAPSTTL |
| | SEQ ID NO: | TLRSKSMTAELEELASIRSSEEEPELVFAVNLPPAQLSSSDEETREEL |
| | 2 | ARIGLVPPPEEFANGVLLATPLAGPGPSPTTVPSPASGKPSSEPPPAPE |
| | | SAADSGVEEADTRSSSDPHLETTSTISTVSSMSTLSSESGELTDTHTSF |
| | | ADGHTFLLEKPPVPPKPKLKSPLGKGPVTFRDPLLKQSSDSELMAQQ |
| | | HHAASAGLASAAGPARPRYLFQRRSKLWGDPVESRGLPGPEDDKPT |
| | | VISELSSRLQQLNKDTRSLGEEPVGGLGSLLDPAKKSPIAAARLFSSL |
| | | GELSSISAQRSPGGPGGGASYSVRPSGRYPVARRAPSPVKPASLERV |
| | | EGLGAGAGGAGRPFGLTPPTILKSSSLSIPHEPKEVRFVVRSVSARSR |
| | | SPSPSPLPSPASGPGPGAPGPRRPFQQKPLQLWSKFDVGDWLESIHL |
| | | GEHRDRFEDHEIEGAHLPALTKDDFVELGVTRVGHRMNIERALRQL |
| | | DGS* |
| 19 | Mouse | MDGPGASAVVVRVGIPDLQQTKCLRLDPTAPVWAAKQRVLCALNH |
| | miniShank3- | SLQDALNYGLFQPPSRGRAGKFLDEERLLQDYPPNLDTPLPYLEFRY |
| | V2 protein | KRRVYAQNLIDDKQFAKLHTKANLKKFMDYVQLHSTDKVARLLD |
| | sequence | KGLDPNFHDPDSGECPLSLAAQLDNATDLLKVLRNGGAHLDFRTRD |
| | encoded by | GLTAVHCATRQRNAGALTTLLDLGASPDYKDSRGLTPLYHSALGG |
| | SEQ ID NO: | GDALCCELLLHDHAQLGTTDENGWQEIHQACRFGHVQHLEHLLFY |
| | 3 | GANMGAQNASGNTALHICALYNQESCARVLLFRGANKDVRNYNS |
| | | QTAFQVAIIAGNFELAEVIKTHKDSDVVPFRETPSYAKRRRLAGPSG |
| | | LASPRPLQRSASDINLKGAPPPRGPKRKLYSAVPGRKFIAVKAHSPQ |
| | | GEGEIPLHRGEAVKVLSIGEGGFWEGTVKGRTGWFPADCVEEVQM |
| | | RQYDTRHETREDRTKRLFRHYTVGSYDSLTSHSDYVIDDKVAILQK |
| | | RDHEGFGFVLRGAKAETPIEEFTPTPAFPALQYLESVDVEGVAWRA |
| | | GLRTGDFLIEVNGVNVVKVGHKQVVGLIRQGGNRLVMKVVSVTRK |
| | | PEEDGARRRAPPPPKRAPSTTLTLRSKSMTAELEELASIRSSEEEPEL |
| | | VFAVNLPPAQLSSSDEETREELARIGLVPPPEEFANGILLTTPPPGPGP |
| | | LPTTVPSPASGKPSSELPPAPESAADSGVEEADTRSSSDPHLETTSTIS |
| | | TVSSMSTLSSESGELTDTHTSFADGHTFLLEKPPVPPKPKLKSPLGKG |
| | | PVTFRDPLLKQSSDSELMAQQHHAASTGLASAAGPARPRYLFQRRS |
| | | KLWGDPVESRGLPGPEDDKPTVISELSSRLQQLNKDTRSLGEEPVGG |
| | | LGSLLDPAKKSPIAAARLFSSLGELSTISAQRSPGGPGGGASYSVRPS |
| | | GRYPVARRAPSPVKPASLERVEGLGAGVGGAGRPFGLTPPTILKSSS |
| | | LSIPHEPKEVRFVVRSVSARSRSPSPSPLPSPSPGSGPSAGPRRPFQQK |
| | | PLQLWSKFDVGDWLESIHLGEHRDRFEDHEIEGAHLPALTKEDFVE |
| | | LGVTRVGHRMNIERALRQLDGS* |
| 20 | Human | MDGPGASAVVVRVGIPDLQQTKCLRLDPAAPVWAAKQRVLCALN |
| | miniShank3- | HSLQDALNYGLFQPPSRGRAGKFLDEERLLQEYPPNLDTPLPYLEFR |
| | V2 protein | YKRRVYAQNLIDDKQFAKLHTKANLKKFMDYVQLHSTDKVARLL |
| | sequence | DKGLDPNFHDPDSGECPLSLAAQLDNATDLLKVLKNGGAHLDFRT |
| | encoded by | RDGLTAVHCATRQRNAAALTTLLDLGASPDYKDSRGLTPLYHSAL |
| | SEQ ID NO: | GGGDALCCELLLHDHAQLGITDENGWQEIHQACRFGHVQHLEHLL |
| | 4 | FYGADMGAQNASGNTALHICALYNQESCARVLLFRGANRDVRNYN |
| | | SQTAFQVAIIAGNFELAEVIKTHKDSDVVPFRETPSYAKRRRLAGPS |
| | | GLASPRPLQRSASDINLKGAPPPRGPKRKLYSAVPGRKFIAVKAHSP |
| | | QGEGEIPLHRGEAVKVLSIGEGGFWEGTVKGRTGWFPADCVEEVQ |
| | | MRQHDTRPETREDRTKRLFRHYTVGSYDSLTSHSDYVIDDKVAVLQ |
| | | KRDHEGFGFVLRGAKAETPIEEFTPTPAFPALQYLESVDVEGVAWR |
| | | AGLRTGDFLIEVNGVNVVKVGHKQVVALIRQGGNRLVMKVVSVTR |
| | | KPEEDGARRRAPPPPKRAPSTTLTLRSKSMTAELEELASIRSSEEEPE |
| | | LVFAVNLPPAQLSSSDEETREELARIGLVPPPEEFANGVLLATPLAGP |
| | | GPSPTTVPSPASGKPSSEPPPAPESAADSGVEEADTRSSSDPHLETTST |
| | | ISTVSSMSTLSSESGELTDTHTSFADGHTFLLEKPPVPPKPKLKSPLG |
| | | KGPVTFRDPLLKQSSDSELMAQQHHAASAGLASAAGPARPRYLFQR |
| | | RSKLWGDPVESRGLPGPEDDKPTVISELSSRLQQLNKDTRSLGEEPV |
| | | GGLGSLLDPAKKSPIAAARLFSSLGELSSISAQRSPGGPGGGASYSVR |
| | | PSGRYPVARRAPSPVKPASLERVEGLGAGAGGAGRPFGLTPPTILKS |
| | | SSLSIPHEPKEVRFVVRSVSARSRSPSPSPLPSPASGPGPGAPGPRRPF |
| | | QQKPLQLWSKFDVGDWLESIHLGEHRDRFEDHEIEGAHLPALTKDD |
| | | FVELGVTRVGHRMNIERALRQLDGS* |
| 21 | AAV-hSyn1- | GGGGTGCCTACCTGACGACCGACCCCGACCCACTGGACAAGCAC |
| | HumanMiniS | CCTGCAGGCAGCTGCGCGCTCGCTCGCTCACTGAGGCCGCCCGG |
| | hank3-V1 | GCGTCGGGCGACCTTTGGTCGCCCGGCCTCAGTGAGCGAGCGAG |
| | (5′ITR-hSyn- | CGCGCAGAGAGGGAGTGGCCAACTCCATCACTAGGGGTTCCTGC |
| | WPRE-hgH | GGCCGCACGCGTTTAATTAAGTGTCTAGACTGCAGAGGGCCCTG |
| | polyA-3′ITR) | CGTATGAGTGCAAGTGGGTTTTAGGACCAGGATGAGGCGGGGTG |
| | | CCAACCCCCATTCCCCAAATTGCGCATCCCCTATCAGAGAGGGG |
| | | GAGGGGAAACAGGATGCGGCGAGGCGCGTGCGCACTGCCAGCTT |
| | | CAGCACCGCGGACAGTGCCTTCGCCCCCGCCTGGCGGCGCGCGC |
| | | CACCGCCGCCTCAGCACTGAAGGCGCGCTGACGTCACTCGCCGG |
| | | TCCCCCGCAAACTCCCCTTCCCGGCCACCTTGGTCGCGTCCGCGC |
| | | CGCCGCCGGCCCAGCCGGACCGCACCACGCGAGGCGCGAGATA |
| | | GGGGGGCACGGGCGCGACCATCTGCGCTGCGGCGCCGGCGACTC |
| | | AGCGCTGCCTCAGTCTGCGGTGGGCAGCGGAGGAGTCGTGTCGT |
| | | GCCTGAGAGCGCAGTCGAGAAACCGGCTAGAGGATCCTTCGAAA |
| | | CCGGTGCTAGCACCATGGGCCCGAAGCGGAAACTTTACAGCGCC |
| | | GTCCCCGGCCGCAAGTTCATCGCCGTGAAGGCGCACAGCCCGCA |
| | | GGGTGAAGGCGAGATCCCGCTGCACCGCGGCGAGGCCGTGAAG |
| | | GTGCTCAGCATTGGGGAGGGCGGTTTCTGGGAGGGAACCGTGAA |
| | | AGGCCGCACGGGCTGGTTCCCGGCCGACTGCGTGGAGGAAGTGC |
| | | AGATGAGGCAGCATGACACACGGCCTGAAACGCGGGAGGACCG |
| | | GACGAAGCGGCTCTTTCGGCACTACACAGTGGGCTCCTACGACA |
| | | GCCTCACCTCACACAGCGATTATGTCATTGATGACAAAGTGGCTG |
| | | TCCTGCAGAAACGGGACCACGAGGGCTTTGGTTTTGTGCTCCGG |
| | | GGAGCCAAAGCAGAGACCCCCATCGAGGAGTTCACGCCCACGCC |
| | | AGCCTTCCCGGCGCTGCAGTATCTCGAGTCGGTGGACGTGGAGG |
| | | GTGTGGCCTGGAGGGCCGGGCTGCGCACGGGAGACTTCCTCATC |
| | | GAGGTGAACGGGGTGAACGTGGTGAAGGTCGGACACAAGCAGG |
| | | TGGTGGCTCTGATTCGCCAGGGTGGCAACCGCCTCGTCATGAAG |
| | | GTTGTGTCTGTGACAAGGAAGCCAGAAGAGGACGGGGCTCGGCG |
| | | CAGAGCCCCACCGCCCCCCAAGAGGGCCCCCAGCACCACACTGA |
| | | CCCTGCGCTCCAAGTCCATGACAGCTGAGCTCGAGGAACTTGCC |
| | | TCCATTCGGAGCTCAGAGGAAGAGCCAGAGCTGGTGTTTGCTGT |
| | | GAACCTGCCACCTGCCCAGCTGTCGTCCAGCGATGAGGAGACCA |
| | | GGGAGGAGCTGGCCCGAATTGGGTTGGTGCCACCCCCTGAAGAG |
| | | TTTGCCAACGGGGTCCTGCTGGCCACCCCACTCGCTGGCCCGGGC |
| | | CCCTCGCCCACCACGGTGCCCAGCCCGGCCTCAGGGAAGCCCAG |
| | | CAGTGAGCCACCCCCTGCCCCTGAGTCTGCAGCCGACTCTGGGG |
| | | TGGAGGAGGCTGACACACGCAGCTCCAGCGACCCCCACCTGGAG |
| | | ACCACAAGCACCATCTCCACGGTGTCCAGCATGTCCACCTTGAG |
| | | CTCGGAGAGCGGGGAACTCACTGACACCCACACCTCCTTCGCTG |
| | | ACGGACACACTTTTCTACTCGAGAAGCCACCAGTGCCTCCCAAG |
| | | CCCAAGCTCAAGTCCCCGCTGGGGAAGGGGCCGGTGACCTTCAG |
| | | GGACCCGCTGCTGAAGCAGTCCTCGGACAGCGAGCTCATGGCCC |
| | | AGCAGCACCACGCCGCCTCTGCCGGGCTGGCCTCTGCCGCCGGG |
| | | CCTGCCCGCCCTCGCTACCTCTTCCAGAGAAGGTCCAAGCTATGG |
| | | GGGGACCCCGTGGAGAGCCGGGGGCTCCCTGGGCCTGAAGACG |
| | | ACAAACCAACTGTGATCAGTGAGCTCAGCTCCCGCCTGCAGCAG |
| | | CTGAACAAGGACACGCGTTCCCTGGGGGAGGAACCAGTTGGTGG |
| | | CCTGGGCAGCCTGCTGGACCCTGCCAAGAAGTCGCCCATCGCAG |
| | | CAGCTCGGCTCTTCAGCAGCCTCGGTGAGCTGAGCTCCATTTCAG |
| | | CGCAGCGCAGCCCCGGGGGCCCGGGCGGCGGGGCCTCGTACTCG |
| | | GTGAGGCCCAGTGGCCGCTACCCCGTGGCGAGACGCGCCCCGAG |
| | | CCCGGTGAAGCCCGCGTCGCTGGAGCGGGTGGAGGGGCTGGGGG |
| | | CGGGCGCGGGGGGCGCAGGGCGGCCCTTCGGCCTCACGCCCCCC |
| | | ACCATCCTCAAGTCGTCCAGCCTCTCCATCCCGCACGAGCCCAAG |
| | | GAGGTGCGCTTCGTGGTGCGCAGCGTGAGCGCGCGCAGTCGCTC |
| | | CCCCTCGCCGTCGCCGCTGCCCTCGCCCGCGTCCGGCCCCGGCCC |
| | | CGGCGCCCCCGGCCCACGCCGACCCTTCCAGCAGAAGCCGCTGC |
| | | AGCTCTGGAGCAAGTTCGACGTGGGCGACTGGCTGGAGAGCATC |
| | | CACCTAGGCGAGCACCGCGACCGCTTCGAGGACCATGAGATAGA |
| | | AGGCGCGCACCTACCCGCGCTTACCAAGGACGACTTCGTGGAGC |
| | | TGGGCGTCACGCGCGTGGGCCACCGCATGAACATCGAGCGCGCG |
| | | CTCAGGCAGCTGGACGGCAGCTGACGGCGCGCCACCGGTCAAAC |
| | | ACCATTGTCACACTCCATACTATCAAACACCATTGTCACACTCCA |
| | | GATCAACAAACACCATTGTCACACTCCACGCCGGCGGGTACCAG |
| | | TGAATTCTACCAGTGCCATAGATCAAAGTGAATTCTACCAGTGCC |
| | | ATATACTATAGTGAATTCTACCAGTGCCATACAAGCTTATCGATA |
| | | ATCAACCTCTGGATTACAAAATTTGTGAAAGATTGACTGGTATTC |
| | | TTAACTATGTTGCTCCTTTTACGCTATGTGGATACGCTGCTTTAAT |
| | | GCCTTTGTATCATGCTATTGCTTCCCGTATGGCTTTCATTTTCTCC |
| | | TCCTTGTATAAATCCTGGTTAGTTCTTGCCACGGCGGAACTCATC |
| | | GCCGCCTGCCTTGCCCGCTGCTGGACAGGGGCTCGGCTGTTGGG |
| | | CACTGACAATTCCGTGGTGTTAGCGCTGCTCGAGAGATCTACGG |
| | | GTGGCATCCCTGTGACCCCTCCCCAGTGCCTCTCCTGGCCCTGGA |
| | | AGTTGCCACTCCAGTGCCCACCAGCCTTGTCCTAATAAAATTAAG |
| | | TTGCATCATTTTGTCTGACTAGGTGTCCTTCTATAATATTATGGGG |
| | | TGGAGGGGGGTGGTATGGAGCAAGGGGCAAGTTGGGAAGACAA |
| | | CCTGTAGGGCCTGCGGGGTCTATTGGGAACCACACGTGCGGACC |
| | | GAGCGGCCGCAGGAACCCCTAGTGATGGAGTTGGCCACTCCCTC |
| | | TCTGCGCGCTCGCTCGCTCACTGAGGCCGGGCGACCAAAGGTCG |
| | | CCCGACGCCCGGGCTTTGCCCGGGCGGCCTCAGTGAGCGAGCGA |
| | | GCGCGCAGCTGCCTGCAGGGGCGCCTGATGCGGTATTTTCTCCTT |
| | | ACGCATCTGTGCGGTATTTCACACCGCATACGTCAAAGCAACCAT |
| | | AGTACGCGCCCTGTAGCGGCGCATTAAGCGCGGCGGGTGTGGTG |
| | | GTTACGCGCAGCGTGACCGCTACACTTGCCAGCGCCTTAGCGCC |
| | | CGCTCCTTTCGCTTTCTTCCCTTCCTTTCTCGCCACGTTCGCCGGC |
| | | TTTCCCCGTCAAGCTCTAAATCGGGGGCTCCCTTTAGGGTTCCGA |
| | | TTTAGTGCTTTACGGCACCTCGACCCCAAAAAACTTGATTTGGGT |
| | | GATGGTTCACGTAGTGGGCCATCGCCCTGATAGACGGTTTTTCGC |
| | | CCTTTGACGTTGGAGTCCACGTTCTTTAATAGTGGACTCTTGTTCC |
| | | AAACTGGAACAACACTCAACTCTATCTCGGGCTATTCTTTTGATT |
| | | TATAAGGGATTTTGCCGATTTCGGTCTATTGGTTAAAAAATGAGC |
| | | TGATTTAACAAAAATTTAACGCGAATTTTAACAAAATATTAACGT |
| | | TTACAATTTTATGGTGCACTCTCAGTACAATCTGCTCTGATGCCG |
| | | CATAGTTAAGCCAGCCCCGACACCCGCCAACACCCGCTGACGCG |
| | | CCCTGACGGGCTTGTCTGCTCCCGGCATCCGCTTACAGACAAGCT |
| | | GTGACCGTCTCCGGGAGCTGCATGTGTCAGAGGTTTTCACCGTCA |
| | | TCACCGAAACGCGCGAGACGAAAGGGCCTCGTGATACGCCTATT |
| | | TTTATAGGTTAATGTCATGATAATAATGGTTTCTTAGACGTCAGG |
| | | TGGCACTTTTCGGGGAAATGTGCGCGGAACCCCTATTTGTTTATT |
| | | TTTCTAAATACATTCAAATATGTATCCGCTCATGAGACAATAACC |
| | | CTGATAAATGCTTCAATAATATTGAAAAAGGAAGAGTATGAGTA |
| | | TTCAACATTTCCGTGTCGCCCTTATTCCCTTTTTTGCGGCATTTTG |
| | | CCTTCCTGTTTTTGCTCACCCAGAAACGCTGGTGAAAGTAAAAGA |
| | | TGCTGAAGATCAGTTGGGTGCACGAGTGGGTTACATCGAACTGG |
| | | ATCTCAACAGCGGTAAGATCCTTGAGAGTTTTCGCCCCGAAGAA |
| | | CGTTTTCCAATGATGAGCACTTTTAAAGTTCTGCTATGTGGCGCG |
| | | GTATTATCCCGTATTGACGCCGGGCAAGAGCAACTCGGTCGCCG |
| | | CATACACTATTCTCAGAATGACTTGGTTGAGTACTCACCAGTCAC |
| | | AGAAAAGCATCTTACGGATGGCATGACAGTAAGAGAATTATGCA |
| | | GTGCTGCCATAACCATGAGTGATAACACTGCGGCCAACTTACTTC |
| | | TGACAACGATCGGAGGACCGAAGGAGCTAACCGCTTTTTTGCAC |
| | | AACATGGGGGATCATGTAACTCGCCTTGATCGTTGGGAACCGGA |
| | | GCTGAATGAAGCCATACCAAACGACGAGCGTGACACCACGATGC |
| | | CTGTAGCAATGGCAACAACGTTGCGCAAACTATTAACTGGCGAA |
| | | CTACTTACTCTAGCTTCCCGGCAACAATTAATAGACTGGATGGAG |
| | | GCGGATAAAGTTGCAGGACCACTTCTGCGCTCGGCCCTTCCGGCT |
| | | GGCTGGTTTATTGCTGATAAATCTGGAGCCGGTGAGCGTGGGTCT |
| | | CGCGGTATCATTGCAGCACTGGGGCCAGATGGTAAGCCCTCCCG |
| | | TATCGTAGTTATCTACACGACGGGGAGTCAGGCAACTATGGATG |
| | | AACGAAATAGACAGATCGCTGAGATAGGTGCCTCACTGATTAAG |
| | | CATTGGTAACTGTCAGACCAAGTTTACTCATATATACTTTAGATT |
| | | GATTTAAAACTTCATTTTTAATTTAAAAGGATCTAGGTGAAGATC |
| | | CTTTTTGATAATCTCATGACCAAAATCCCTTAACGTGAGTTTTCG |
| | | TTCCACTGAGCGTCAGACCCCGTAGAAAAGATCAAAGGATCTTC |
| | | TTGAGATCCTTTTTTTCTGCGCGTAATCTGCTGCTTGCAAACAAA |
| | | AAAACCACCGCTACCAGCGGTGGTTTGTTTGCCGGATCAAGAGC |
| | | TACCAACTCTTTTTCCGAAGGTAACTGGCTTCAGCAGAGCGCAGA |
| | | TACCAAATACTGTTCTTCTAGTGTAGCCGTAGTTAGGCCACCACT |
| | | TCAAGAACTCTGTAGCACCGCCTACATACCTCGCTCTGCTAATCC |
| | | TGTTACCAGTGGCTGCTGCCAGTGGCGATAAGTCGTGTCTTACCG |
| | | GGTTGGACTCAAGACGATAGTTACCGGATAAGGCGCAGCGGTCG |
| | | GGCTGAACGGGGGGTTCGTGCACACAGCCCAGCTTGGAGCGAAC |
| | | GACCTACACCGAACTGAGATACCTACAGCGTGAGCTATGAGAAA |
| | | GCGCCACGCTTCCCGAAGGGAGAAAGGCGGACAGGTATCCGGTA |
| | | AGCGGCAGGGTCGGAACAGGAGAGCGCACGAGGGAGCTTCCAG |
| | | GGGGAAACGCCTGGTATCTTTATAGTCCTGTCGGGTTTCGCCACC |
| | | TCTGACTTGAGCGTCGATTTTTGTGATGCTCGTCAGGGGGGCGGA |
| | | GCCTATGGAAAAACGCCAGCAACGCGGCCTTTTTACGGTTCCTG |
| | | GCCTTTTGCTGGCCTTTTGCTCACATGT |
| 22 | hSyn1 | gtgtctagactgcagagggccctgcgtatgagtgcaagtgggttttaggaccaggatgaggcggggtggg |
| | promoter | ggtgcctacctgacgaccgaccccgacccactggacaagcacccaacccccattccccaaattgcgcatc |
| | | ccctatcagagagggggggggaaacaggatgcggcgaggcgcgtgcgcactgccagcttcagcaccg |
| | | cggacagtgccttcgcccccgcctggcggcgcgcgccaccgccgcctcagcactgaaggcgcgctgac |
| | | gtcactcgccggtcccccgcaaactccccttcccggccaccttggtcgcgtccgcgccgccgccggccca |
| | | gccggaccgcaccacgcgaggcgcgagataggggggcacgggcgcgaccatctgcgctgcggcgcc |
| | | ggcgactcagcgctgcctcagtctgcggtgggcagcggaggagtcgtgtcgtgcctgagagcgcagtcg |
| | | agaaaccggctaga |

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists (e.g., in Markush group format), each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included in such ranges unless otherwise specified. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the disclosure, as defined in the following claims. The entire disclosures of PCT Publication No. WO2022/040239 and US Publication No. 2023-0340041 are incorporated by reference herein in their entirety.

1. A method of treating an autism spectrum disorder (ASD) or Phelan-McDermid syndrome in a primate subject in need thereof, the method comprising administering to the primate subject a therapeutically effective amount of a composition comprising an AAV vector, wherein the AAV vector comprises a polynucleotide encoding a Shank3 protein.

2. The method of claim 1, wherein the composition is administered by intravenous administration or intracerebroventricular administration.

3. (canceled)

4. The method of claim 1, wherein the primate subject is 10 years old or younger.

5. (canceled)

6. The method of claim 1, wherein the Shank3 protein is a miniShank3 protein.

7. The method of claim 6, wherein the miniShank3 protein comprises a sequence that is at least 80%, at least 85%, at least 90%, or at least 95% identical to SEQ ID NO: 18 or 20.

8. (canceled)

9. The method of claim 1, wherein administering the composition results in an improvement in one or more symptoms of ASD.

10. The method of claim 9, wherein the one or more symptoms of ASD are selected from: atypical behavior, atypical social behavior, cognitive deficits, communication deficits, and/or abnormal connectivity.

11.-12. (canceled)

13. A method of treating or ameliorating symptoms of an autism spectrum disorder (ASD) or Phelan-McDermid syndrome or a syndrome associated with a SHANK3 mutation in a primate subject in need thereof, the method comprising administering to the primate subject a therapeutically effective amount of a composition comprising an AAV vector, wherein the AAV vector comprises a) an artificial genome comprising a polynucleotide encoding a transgene comprising a miniShank3 protein comprising an amino acid sequence at least 90% identical to SEQ ID NO: 18 or SEQ ID NO: 20 and has SHANK3 activity, wherein the transgene is operably linked to an hSYN promoter comprising SEQ ID NO: 22 and b) a capsid protein, wherein the capsid protein is AAV9 or a variant thereof.

14. The method of claim 13, wherein the primate subject has at about 3 months, about 6 months or about one year after said administering a decreased response to negative stimuli compared to the primate subject before treatment or a similarly situated untreated control primate.

15. The method of claim 13, wherein the primate subject has at about 3 months, about 6 months or about one year after said administering increased motivation, flexibility, and memory compared to the primate subject before treatment or a similarly situated untreated control primate.

16. The method of claim 13, wherein the primate subject at about 3 months, about 6 months or about one year after said administering has an increased response to reward compared to the primate subject before treatment or a similarly situated untreated control primate.

17. The method of claim 13, wherein the primate subject at about 3 months, about 6 months or about one year after said administering has decreased cognitive deficit and/or intellectual disability compared to the primate subject before treatment or a similarly situated untreated control primate.

18. The method of claim 13, wherein the primate subject at about 3 months, about 6 months or about one year after said administering has increased resting fMRI connectivity lower in anterior/medial areas compared to the primate subject before treatment or a similarly situated untreated control primate.

19. The method of claim 13, wherein the primate subject at about 3 months, about 6 months or about one year after said administering has decreased resting fMRI connectivity lower in posterior/lateral (sensory) areas compared to the primate subject before treatment or a similarly situated untreated control primate.

20.-24. (canceled)

25. The method of claim 13, wherein the administration is intravenous administration or intracerebroventricular administration.

26.-28. (canceled)

29. The method of claim 13, wherein the capsid protein or variant thereof crosses the blood-brain barrier.

30. The method of claim 1, wherein the polynucleotide comprises a sequence that is at least 80%, at least 85%, at least 90%, or at least 95% identical to SEQ ID NO: 21.

31. The method of claim 30, wherein the polynucleotide comprises a sequence that is 100% identical to SEQ ID NO: 21.

32. The method of claim 13, wherein the polynucleotide comprises a sequence that is at least 80%, at least 85%, at least 90%, or at least 95% identical to SEQ ID NO: 21.

33. The method of claim 32, wherein the polynucleotide comprises a sequence that is 100% identical to SEQ ID NO: 21.


SEQUENCE LISTING (22 sequences)

SEQ ID NO: 1 - DNA, 2121 bp, Mus musculus
  source 1..2121: mol_type = genomic DNA
atgggcccgaagcggaaactttacagtgccgtccccggccgcaagttcatcgctgtgaaggcgcacagcccgcagggcgagggcgagatcccgctgcaccgcggcgaggccgtgaaggtgctcagcattggggagggcggtttctgggagggaaccgtgaagggccgaacaggctggttcccagctgactgtgtggaagaagtgcagatgcgacagtatgacacccggcatgaaaccagagaggaccggacgaagcgtctcttccgccactacactgtgggttcctatgacagcctcacttcacacagcgattatgtcatcgatgataaggtggctatcctgcagaaaagggaccatgaggggtttggctttgttctccggggagccaaagcagagacccccattgaggagtttacacccacacctgccttccctgcactccaataccttgagtctgtagatgtggaaggtgtggcctggagggctggacttcgaactggggacttcctcattgaggtgaacggagtgaatgtcgtgaaggttggacacaagcaagtggtgggtctcatccgtcagggtggcaaccgcctggtcatgaaggttgtgtctgtgaccaggaaacccgaggaggatggtgctcggcgcagagccccaccacccccaaagagggctcccagcaccacgctgaccctgcggtccaagtccatgacggctgagctcgaggaacttgcttccattcggagctcagaggaggagccagagctggtattcgctgtgaacctgccacctgctcagctgtcctccagcgatgaggagaccagagaggagctggcccgcatagggctagtgccaccccctgaagagtttgccaatgggatcctgctgaccaccccgcccccagggccgggccccttgcccaccacggtacccagcccggcctcagggaagcccagcagcgagctgccccctgcccctgagtctgcagctgactctggagtagaggaggctgacactcgaagctccagtgacccccacctggagaccacaagcaccatttccacagtgtccagcatgtccaccctgagctcggagagtggggaactcacggacacccacacctcctttgccgatggacacacttttctactcgagaagccaccagtgcctcccaagcccaagctcaagtccccgctggggaaggggccggtgaccttcagggacccgctgctgaagcaatcctcggacagtgagctcatggcccagcagcaccatgctgcctctactgggttggcttctgctgctgggcccgcccgccctcgctacctcttccagagaaggtccaagctgtggggggaccccgtggagagtcgggggctccctgggcctgaagatgacaaaccaactgtgatcagtgagctcagctcccgtctgcagcagctgaataaagacacacgctccttgggggaggaaccagttggtggcctgggcagcctgctggaccctgctaagaagtcacccattgcagcagctcggctcttcagcagcctcggtgagctgagcaccatctcagcgcagcgcagcccggggggcccgggcggaggggcctcctactcggtgcggcccagcggccggtaccccgtggcgagacgagccccgagcccagtgaaacccgcatcgctggagcgggtggaggggctgggggcgggcgtgggaggcgcggggcggcccttcggcctcacgcctcccaccatcctcaagtcgtccagcctctccatcccgcacgaacccaaggaagtgcgcttcgtggtgcgaagtgtgagtgcgcgcagccgctccccctcaccatctccgctgccctcgccttctcccggctctggccccagtgccggcccgcgtcggccatttcaacagaagcccctgcagctctggagcaagttcgatgtgggcgactggctggagagcatccacttaggcgagcaccgagaccgcttcgaggaccatgagatcgaaggcgcacacctgcctgcgctcaccaaggaagacttcgtggagctgggcgtcacacgcgttggccaccgcatgaacatcgagcgtgcgctcaggcagctggatggcagctga

SEQ ID NO: 2 - DNA, 2124 bp, Homo sapiens
  source 1..2124: mol_type = genomic DNA
atgggcccgaagcggaaactttacagcgccgtccccggccgcaagttcatcgccgtgaaggcgcacagcccgcagggtgaaggcgagatcccgctgcaccgcggcgaggccgtgaaggtgctcagcattggggagggcggtttctgggagggaaccgtgaaaggccgcacgggctggttcccggccgactgcgtggaggaagtgcagatgaggcagcatgacacacggcctgaaacgcgggaggaccggacgaagcggctctttcggcactacacagtgggctcctacgacagcctcacctcacacagcgattatgtcattgatgacaaagtggctgtcctgcagaaacgggaccacgagggctttggttttgtgctccggggagccaaagcagagacccccatcgaggagttcacgcccacgccagccttcccggcgctgcagtatctcgagtcggtggacgtggagggtgtggcctggagggccgggctgcgcacgggagacttcctcatcgaggtgaacggggtgaacgtggtgaaggtcggacacaagcaggtggtggctctgattcgccagggtggcaaccgcctcgtcatgaaggttgtgtctgtgacaaggaagccagaagaggacggggctcggcgcagagccccaccgccccccaagagggcccccagcaccacactgaccctgcgctccaagtccatgacagctgagctcgaggaacttgcctccattcggagctcagaggaagagccagagctggtgtttgctgtgaacctgccacctgcccagctgtcgtccagcgatgaggagaccagggaggagctggcccgaattgggttggtgccaccccctgaagagtttgccaacggggtcctgctggccaccccactcgctggcccgggcccctcgcccaccacggtgcccagcccggcctcagggaagcccagcagtgagccaccccctgcccctgagtctgcagccgactctggggtggaggaggctgacacacgcagctccagcgacccccacctggagaccacaagcaccatctccacggtgtccagcatgtccaccttgagctcggagagcggggaactcactgacacccacacctccttcgctgacggacacacttttctactcgagaagccaccagtgcctcccaagcccaagctcaagtccccgctggggaaggggccggtgaccttcagggacccgctgctgaagcagtcctcggacagcgagctcatggcccagcagcaccacgccgcctctgccgggctggcctctgccgccgggcctgcccgccctcgctacctcttccagagaaggtccaagctatggggggaccccgtggagagccgggggctccctgggcctgaagacgacaaaccaactgtgatcagtgagctcagctcccgcctgcagcagctgaacaaggacacgcgttccctgggggaggaaccagttggtggcctgggcagcctgctggaccctgccaagaagtcgcccatcgcagcagctcggctcttcagcagcctcggtgagctgagctccatttcagcgcagcgcagccccgggggcccgggcggcggggcctcgtactcggtgaggcccagtggccgctaccccgtggcgagacgcgccccgagcccggtgaagcccgcgtcgctggagcgggtggaggggctgggggcgggcgcggggggcgcagggcggcccttcggcctcacgccccccaccatcctcaagtcgtccagcctctccatcccgcacgagcccaaggaggtgcgcttcgtggtgcgcagcgtgagcgcgcgcagtcgctccccctcgccgtcgccgctgccctcgcccgcgtccggccccggccccggcgcccccggcccacgccgacccttccagcagaagccgctgcagctctggagcaagttcgacgtgggcgactggctggagagcatccacctaggcgagcaccgcgaccgcttcgaggaccatgagatagaaggcgcgcacctacccgcgcttaccaaggacgacttcgtggagctgggcgtcacgcgcgtgggccaccgcatgaacatcgagcgcgcgctcaggcagctggacggcagctga

SEQ ID NO: 3 - DNA, 3276 bp, Mus musculus
  source 1..3276: mol_type = genomic DNA
atggacggccccggggccagcgccgtggtcgtgcgcgtcggcatcccggacctgcaacaaacgaagtgcctgcgtctggacccaaccgcgcccgtgtgggccgccaagcagcgtgtgctctgcgccctcaatcatagccttcaagacgcgctcaactacgggctattccagcctccctcccggggtcgcgccggcaagttcctggatgaagagcggctcttacaggactacccgcctaacctggacacgcccctgccctatctggagttccgatacaagcggagagtttatgcccagaacctcatagatgacaagcagtttgcaaagctacacacaaaggcaaacctgaagaagttcatggactatgtccagctacacagcacagataaggtggcccgcctgctggacaaggggctggaccccaatttccatgaccctgactcaggagagtgccctctgagccttgcggcacagttggacaacgccactgacctcctgaaggttctccgcaacggcggtgctcatctggacttccggacccgagatgggctgacagccgtccactgtgctacccgccagcggaacgcaggggcattgacgaccctgctggacctgggggcttcgcctgactacaaggacagccgcggcctgacgcccctgtaccatagtgccctagggggcggggatgccctctgttgcgagctgcttctccatgatcatgcacagctggggaccactgatgagaatggttggcaagagatccatcaggcctgtcgctttggacacgtgcagcacctggagcaccttttgttctatggggccaacatgggtgctcagaatgcctcgggaaacacagccctgcacatctgtgccctctacaaccaggagagttgcgcgcgcgtcctgcttttccgtggtgccaacaaggacgtccgcaattacaacagccagacagccttccaggtggccattattgcagggaactttgagcttgccgaggtaatcaagacccacaaagactccgatgtcgtaccattcagggaaacccccagctatgcaaagcgacggcgtctggctggcccgagtggcctggcatccccacggcccttacagcgctcagccagtgatatcaacctgaaaggtgcgccgccgccccggggcccgaagcggaaactttacagtgccgtccccggccgcaagttcatcgctgtgaaggcgcacagcccgcagggcgagggcgagatcccgctgcaccgcggcgaggccgtgaaggtgctcagcattggggagggcggtttctgggagggaaccgtgaagggccgaacaggctggttcccagctgactgtgtggaagaagtgcagatgcgacagtatgacacccggcatgaaaccagagaggaccggacgaagcgtctcttccgccactacactgtgggttcctatgacagcctcacttcacacagcgattatgtcatcgatgataaggtggctatcctgcagaaaagggaccatgaggggtttggctttgttctccggggagccaaagcagagacccccattgaggagtttacacccacacctgccttccctgcactccaataccttgagtctgtagatgtggaaggtgtggcctggagggctggacttcgaactggggacttcctcattgaggtgaacggagtgaatgtcgtgaaggttggacacaagcaagtggtgggtctcatccgtcagggtggcaaccgcctggtcatgaaggttgtgtctgtgaccaggaaacccgaggaggatggtgctcggcgcagagccccaccacccccaaagagggctcccagcaccacgctgaccctgcggtccaagtccatgacggctgagctcgaggaacttgcttccattcggagctcagaggaggagccagagctggtattcgctgtgaacctgccacctgctcagctgtcctccagcgatgaggagaccagagaggagctggcccgcatagggctagtgccaccccctgaagagtttgccaatgggatcctgctgaccaccccgcccccagggccgggccccttgcccaccacggtacccagcccggcctcagggaagcccagcagcgagctgccccctgcccctgagtctgcagctgactctggagtagaggaggctgacactcgaagctccagtgacccccacctggagaccacaagcaccatttccacagtgtccagcatgtccaccctgagctcggagagtggggaactcacggacacccacacctcctttgccgatggacacacttttctactcgagaagccaccagtgcctcccaagcccaagctcaagtccccgctggggaaggggccggtgaccttcagggacccgctgctgaagcaatcctcggacagtgagctcatggcccagcagcaccatgctgcctctactgggttggcttctgctgctgggcccgcccgccctcgctacctcttccagagaaggtccaagctgtggggggaccccgtggagagtcgggggctccctgggcctgaagatgacaaaccaactgtgatcagtgagctcagctcccgtctgcagcagctgaataaagacacacgctccttgggggaggaaccagttggtggcctgggcagcctgctggaccctgctaagaagtcacccattgcagcagctcggctcttcagcagcctcggtgagctgagcaccatctcagcgcagcgcagcccggggggcccgggcggaggggcctcctactcggtgcggcccagcggccggtaccccgtggcgagacgagccccgagcccagtgaaacccgcatcgctggagcgggtggaggggctgggggcgggcgtgggaggcgcggggcggcccttcggcctcacgcctcccaccatcctcaagtcgtccagcctctccatcccgcacgaacccaaggaagtgcgcttcgtggtgcgaagtgtgagtgcgcgcagccgctccccctcaccatctccgctgccctcgccttctcccggctctggccccagtgccggcccgcgtcggccatttcaacagaagcccctgcagctctggagcaagttcgatgtgggcgactggctggagagcatccacttaggcgagcaccgagaccgcttcgaggaccatgagatcgaaggcgcacacctgcctgcgctcaccaaggaagacttcgtggagctgggcgtcacacgcgttggccaccgcatgaacatcgagcgtgcgctcaggcagctggatggcagctga

SEQ ID NO: 4 - DNA, 3279 bp, Homo sapiens
  source 1..3279: mol_type = genomic DNA
atggacggccccggggccagcgccgtggtcgtgcgcgtcggcatcccggacctgcagcagacgaagtgcctgcgcctggacccggccgcgcccgtgtgggccgccaagcagcgcgtgctctgcgccctcaaccacagcctccaggacgcgctcaactatgggcttttccagccgccctcccggggccgcgccggcaagttcctggatgaggagcggctcctgcaggagtacccgcccaacctggacacgcccctgccctacctggagtttcgatacaagcggcgagtttatgcccagaacctcatcgatgataagcagtttgcaaagcttcacacaaaggcgaacctgaagaagttcatggactacgtccagctgcatagcacggacaaggtggcacgcctgttggacaaggggctggaccccaacttccatgaccctgactcaggagagtgccccctgagcctcgcagcccagctggacaacgccacggacctgctaaaggtgctgaagaatggtggtgcccacctggacttccgcactcgcgatgggctcactgccgtgcactgtgccacacgccagcggaatgcggcagcactgacgaccctgctggacctgggggcttcacctgactacaaggacagccgcggcttgacacccctctaccacagcgccctggggggtggggatgccctctgctgtgagctgcttctccacgaccacgctcagctggggatcaccgacgagaatggctggcaggagatccaccaggcctgccgctttgggcacgtgcagcatctggagcacctgctgttctatggggcagacatgggggcccagaacgcctcggggaacacagccctgcacatctgtgccctctacaaccaggagagctgtgctcgtgtcctgctcttccgtggagctaacagggatgtccgcaactacaacagccagacagccttccaggtggccatcatcgcagggaactttgagcttgcagaggttatcaagacccacaaagactcggatgttgtaccattcagggaaacccccagctatgcgaagcggcggcgactggctggccccagtggcttggcatcccctcggcctctgcagcgctcagccagcgatatcaacctgaagggggcaccgccgccccggggcccgaagcggaaactttacagcgccgtccccggccgcaagttcatcgccgtgaaggcgcacagcccgcagggtgaaggcgagatcccgctgcaccgcggcgaggccgtgaaggtgctcagcattggggagggcggtttctgggagggaaccgtgaaaggccgcacgggctggttcccggccgactgcgtggaggaagtgcagatgaggcagcatgacacacggcctgaaacgcgggaggaccggacgaagcggctctttcggcactacacagtgggctcctacgacagcctcacctcacacagcgattatgtcattgatgacaaagtggctgtcctgcagaaacgggaccacgagggctttggttttgtgctccggggagccaaagcagagacccccatcgaggagttcacgcccacgccagccttcccggcgctgcagtatctcgagtcggtggacgtggagggtgtggcctggagggccgggctgcgcacgggagacttcctcatcgaggtgaacggggtgaacgtggtgaaggtcggacacaagcaggtggtggctctgattcgccagggtggcaaccgcctcgtcatgaaggttgtgtctgtgacaaggaagccagaagaggacggggctcggcgcagagccccaccgccccccaagagggcccccagcaccacactgaccctgcgctccaagtccatgacagctgagctcgaggaacttgcctccattcggagctcagaggaagagccagagctggtgtttgctgtgaacctgccacctgcccagctgtcgtccagcgatgaggagaccagggaggagctggcccgaattgggttggtgccaccccctgaagagtttgccaacggggtcctgctggccaccccactcgctggcccgggcccctcgcccaccacggtgcccagcccggcctcagggaagcccagcagtgagccaccccctgcccctgagtctgcagccgactctggggtggaggaggctgacacacgcagctccagcgacccccacctggagaccacaagcaccatctccacggtgtccagcatgtccaccttgagctcggagagcggggaactcactgacacccacacctccttcgctgacggacacacttttctactcgagaagccaccagtgcctcccaagcccaagctcaagtccccgctggggaaggggccggtgaccttcagggacccgctgctgaagcagtcctcggacagcgagctcatggcccagcagcaccacgccgcctctgccgggctggcctctgccgccgggcctgcccgccctcgctacctcttccagagaaggtccaagctatggggggaccccgtggagagccgggggctccctgggcctgaagacgacaaaccaactgtgatcagtgagctcagctcccgcctgcagcagctgaacaaggacacgcgttccctgggggaggaaccagttggtggcctgggcagcctgctggaccctgccaagaagtcgcccatcgcagcagctcggctcttcagcagcctcggtgagctgagctccatttcagcgcagcgcagccccgggggcccgggcggcggggcctcgtactcggtgaggcccagtggccgctaccccgtggcgagacgcgccccgagcccggtgaagcccgcgtcgctggagcgggtggaggggctgggggcgggcgcggggggcgcagggcggcccttcggcctcacgccccccaccatcctcaagtcgtccagcctctccatcccgcacgagcccaaggaggtgcgcttcgtggtgcgcagcgtgagcgcgcgcagtcgctccccctcgccgtcgccgctgccctcgcccgcgtccggccccggccccggcgcccccggcccacgccgacccttccagcagaagccgctgcagctctggagcaagttcgacgtgggcgactggctggagagcatccacctaggcgagcaccgcgaccgcttcgaggaccatgagatagaaggcgcgcacctacccgcgcttaccaaggacgacttcgtggagctgggcgtcacgcgcgtgggccaccgcatgaacatcgagcgcgcgctcaggcagctggacggcagctga

SEQ ID NO: 5 - protein, 1730 aa, Mus musculus
MDGPGASAVVVRVGIPDLQQTKCLRLDPTAPVWAAKQRVLCALNHSLQDALNYGLFQPPSRGRAGKFLDEERLLQDYPPNLDTPLPYLEFRYKRRVYAQNLIDDKQFAKLHTKANLKKFMDYVQLHSTDKVARLLDKGLDPNFHDPDSGECPLSLAAQLDNATDLLKVLRNGGAHLDFRTRDGLTAVHCATRQRNAGALTTLLDLGASPDYKDSRGLTPLYHSALGGGDALCCELLLHDHAQLGTTDENGWQEIHQACRFGHVQHLEHLLFYGANMGAQNASGNTALHICALYNQESCARVLLFRGANKDVRNYNSQTAFQVAIIAGNFELAEVIKTHKDSDVVPFRETPSYAKRRRLAGPSGLASPRPLQRSASDINLKGDQPAASPGPTLRSLPHQLLLQRLQEEKDRDRDGELENDISGPSAGRGGHNKISPSGPGGSGPAPGPGPASPAPPAPPPRGPKRKLYSAVPGRKFIAVKAHSPQGEGEIPLHRGEAVKVLSIGEGGFWEGTVKGRTGWFPADCVEEVQMRQYDTRHETREDRTKRLFRHYTVGSYDSLTSHSDYVIDDKVAILQKRDHEGFGFVLRGAKAETPIEEFTPTPAFPALQYLESVDVEGVAWRAGLRTGDFLIEVNGVNVVKVGHKQVVGLIRQGGNRLVMKVVSVTRKPEEDGARRRAPPPPKRAPSTTLTLRSKSMTAELEELASIRRRKGEKLDEILAVAAEPTLRPDIADADSRAATVKQRPTSRRITPAEISSLFERQGLPGPEKLPGSLRKGIPRTKSVGEDEKLASLLEGRFPRSTSMQDTVREGRGIPPPPQTAPPPPPAPYYFDSGPPPTFSPPPPPGRAYDTVRSSFKPGLEARLGAGAAGLYDPSTPLGPLPYPERQKRARSMIILQDSAPEVGDVPRPAPAATPPERPKRRPRPSGPDSPYANLGAFSASLFAPSKPQRRKSPLVKQLQVEDAQERAALAVGSPGPVGGSFAREPSPTHRGPRPGSLDYSSGEGLGLTFGGPSPGPVKERRLEERRRSTVFLSVGAIEGSPPSADLPSLQPSRSIDERLLGTGATTGRDLLLPSPVSALKPLVGGPSLGPSGSTFIHPLTGKPLDPSSPLALALAARERALASQTPSRSPTPVHSPDADRPGPLFVDVQTRDSERGPLASPAFSPRSPAWIPVPARREAEKPPREERKSPEDKKSMILSVLDTSLQRPAGLIVVHATSNGQEPSRLGAEEERPGTPELAPAPMQAAAVAEPMPSPRAQPPGSIPADPGPGQGSSEEEPELVFAVNLPPAQLSSSDEETREELARIGLVPPPEEFANGILLTTPPPGPGPLPTTVPSPASGKPSSELPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASTGLASAAGPARPRYLFQRRSKLWGDPVESRGLPGPEDDKPTVISELSSRLQQLNKDTRSLGEEPVGGLGSLLDPAKKSPIAAARLFSSLGELSTISAQRSPGGPGGGASYSVRPSGRYPVARRAPSPVKPASLERVEGLGAGVGGAGRPFGLTPPTILKSSSLSIPHEPKEVRFVVRSVSARSRSPSPSPLPSPSPGSGPSAGPRRPFQQKPLQLWSKFDVGDWLESIHLGEHRDRFEDHEIEGAHLPALTKEDFVELGVTRVGHRMNIERALRQLDGS

SEQ ID NO: 6 - protein, 1731 aa, Homo sapiens
MDGPGASAVVVRVGIPDLQQTKCLRLDPAAPVWAAKQRVLCALNHSLQDALNYGLFQPPSRGRAGKFLDEERLLQEYPPNLDTPLPYLEFRYKRRVYAQNLIDDKQFAKLHTKANLKKFMDYVQLHSTDKVARLLDKGLDPNFHDPDSGECPLSLAAQLDNATDLLKVLKNGGAHLDFRTRDGLTAVHCATRQRNAAALTTLLDLGASPDYKDSRGLTPLYHSALGGGDALCCELLLHDHAQLGITDENGWQEIHQACRFGHVQHLEHLLFYGADMGAQNASGNTALHICALYNQESCARVLLFRGANRDVRNYNSQTAFQVAIIAGNFELAEVIKTHKDSDVVPFRETPSYAKRRRLAGPSGLASPRPLQRSASDINLKGEAQPAASPGPSLRSLPHQLLLQRLQEEKDRDRDADQESNISGPLAGRAGQSKISPSGPGGPGPAPGPGPAPPAPPAPPPRGPKRKLYSAVPGRKFIAVKAHSPQGEGEIPLHRGEAVKVLSIGEGGFWEGTVKGRTGWFPADCVEEVQMRQHDTRPETREDRTKRLFRHYTVGSYDSLTSHSDYVIDDKVAVLQKRDHEGFGFVLRGAKAETPIEEFTPTPAFPALQYLESVDVEGVAWRAGLRTGDFLIEVNGVNVVKVGHKQVVALIRQGGNRLVMKVVSVTRKPEEDGARRRAPPPPKRAPSTTLTLRSKSMTAELEELASIRRRKGEKLDEMLAAAAEPTLRPDIADADSRAATVKQRPTSRRITPAEISSLFERQGLPGPEKLPGSLRKGIPRTKSVGEDEKLASLLEGRFPRSTSMQDPVREGRGIPPPPQTAPPPPPAPYYFDSGPPPAFSPPPPPGRAYDTVRSSFKPGLEARLGAGAAGLYEPGAALGPLPYPERQKRARSMIILQDSAPESGDAPRPPPAATPPERPKRRPRPPGPDSPYANLGAFSASLFAPSKPQRRKSPLVKQLQVEDAQERAALAVGSPGPGGGSFAREPSPTHRGPRPGGLDYGAGDGPGLAFGGPGPAKDRRLEERRRSTVFLSVGAIEGSAPGADLPSLQPSRSIDERLLGTGPTAGRDLLLPSPVSALKPLVSGPSLGPSGSTFIHPLTGKPLDPSSPLALALAARERALASQAPSRSPTPVHSPDADRPGPLFVDVQARDPERGSLASPAFSPRSPAWIPVPARREAEKVPREERKSPEDKKSMILSVLDTSLQRPAGLIVVHATSNGQEPSRLGGAEEERPGTPELAPAPMQSAAVAEPLPSPRAQPPGGTPADAGPGQGSSEEEPELVFAVNLPPAQLSSSDEETREELARIGLVPPPEEFANGVLLATPLAGPGPSPTTVPSPASGKPSSEPPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASAGLASAAGPARPRYLFQRRSKLWGDPVESRGLPGPEDDKPTVISELSSRLQQLNKDTRSLGEEPVGGLGSLLDPAKKSPIAAARLFSSLGELSSISAQRSPGGPGGGASYSVRPSGRYPVARRAPSPVKPASLERVEGLGAGAGGAGRPFGLTPPTILKSSSLSIPHEPKEVRFVVRSVSARSRSPSPSPLPSPASGPGPGAPGPRRPFQQKPLQLWSKFDVGDWLESIHLGEHRDRFEDHEIEGAHLPALTKDDFVELGVTRVGHRMNIERALRQLDGS

SEQ ID NO: 7 - DNA, 7459 bp, synthetic construct
  source 1..7459: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaattaagtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagaggatccttcgaaaccggtgctagcagcgctgttaacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagcggaggaattcacggcccgaagcggaaactttacagtgccgtccccggccgcaagttcatcgctgtgaaggcgcacagcccgcagggcgagggcgagatcccgctgcaccgcggcgaggccgtgaaggtgctcagcattggggagggcggtttctgggagggaaccgtgaagggccgaacaggctggttcccagctgactgtgtggaagaagtgcagatgcgacagtatgacacccggcatgaaaccagagaggaccggacgaagcgtctcttccgccactacactgtgggttcctatgacagcctcacttcacacagcgattatgtcatcgatgataaggtggctatcctgcagaaaagggaccatgaggggtttggctttgttctccggggagccaaagcagagacccccattgaggagtttacacccacacctgccttccctgcactccaataccttgagtctgtagatgtggaaggtgtggcctggagggctggacttcgaactggggacttcctcattgaggtgaacggagtgaatgtcgtgaaggttggacacaagcaagtggtgggtctcatccgtcagggtggcaaccgcctggtcatgaaggttgtgtctgtgaccaggaaacccgaggaggatggtgctcggcgcagagccccaccacccccaaagagggctcccagcaccacgctgaccctgcggtccaagtccatgacggctgagctcgaggaacttgcttccattcggagctcagaggaggagccagagctggtattcgctgtgaacctgccacctgctcagctgtcctccagcgatgaggagaccagagaggagctggcccgcatagggctagtgccaccccctgaagagtttgccaatgggatcctgctgaccaccccgcccccagggccgggccccttgcccaccacggtacccagcccggcctcagggaagcccagcagcgagctgccccctgcccctgagtctgcagctgactctggagtagaggaggctgacactcgaagctccagtgacccccacctggagaccacaagcaccatttccacagtgtccagcatgtccaccctgagctcggagagtggggaactcacggacacccacacctcctttgccgatggacacacttttctactcgagaagccaccagtgcctcccaagcccaagctcaagtccccgctggggaaggggccggtgaccttcagggacccgctgctgaagcaatcctcggacagtgagctcatggcccagcagcaccatgctgcctctactgggttggcttctgctgctgggcccgcccgccctcgctacctcttccagagaaggtccaagctgtggggggaccccgtggagagtcgggggctccctgggcctgaagatgacaaaccaactgtgatcagtgagctcagctcccgtctgcagcagctgaataaagacacacgctccttgggggaggaaccagttggtggcctgggcagcctgctggaccctgctaagaagtcacccattgcagcagctcggctcttcagcagcctcggtgagctgagcaccatctcagcgcagcgcagcccggggggcccgggcggaggggcctcctactcggtgcggcccagcggccggtaccccgtggcgagacgagccccgagcccagtgaaacccgcatcgctggagcgggtggaggggctgggggcgggcgtgggaggcgcggggcggcccttcggcctcacgcctcccaccatcctcaagtcgtccagcctctccatcccgcacgaacccaaggaagtgcgcttcgtggtgcgaagtgtgagtgcgcgcagccgctccccctcaccatctccgctgccctcgccttctcccggctctggccccagtgccggcccgcgtcggccatttcaacagaagcccctgcagctctggagcaagttcgatgtgggcgactggctggagagcatccacttaggcgagcaccgagaccgcttcgaggaccatgagatcgaaggcgcacacctgcctgcgctcaccaaggaagacttcgtggagctgggcgtcacacgcgttggccaccgcatgaacatcgagcgtgcgctcaggcagctggatggcagctgagcggccatcgtcgacggcgcgccaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 8 - DNA, 5331 bp, synthetic construct
  source 1..5331: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaattaagtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagaggatccttcgaaaccggtgctagcagcgctgttaacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggcgcgcccctgcagggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 9 - DNA, 6369 bp, synthetic construct
  source 1..6369: mol_type = other DNA
tcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaattaagtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagaggatccttcgaaaccggtgctagcaccatgcctaagaagaagagaaaggtatgcaggtcacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgatgttaacgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacggcgcgcccctgcagggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg

SEQ ID NO: 10 - DNA, 5703 bp, synthetic construct
  source 1..5703: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaattaagtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagaggatccttcgaaaccggtgctagcaccatgcctaagaagaagagaaaggtatgcaggtcacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgagttaacgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacggcgcgcccctgcagggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 11 - protein, 950 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSGGIHGPKRKLYSAVPGRKFIAVKAHSPQGEGEIPLHRGEAVKVLSIGEGGFWEGTVKGRTGWFPADCVEEVQMRQYDTRHETREDRTKRLFRHYTVGSYDSLTSHSDYVIDDKVAILQKRDHEGFGFVLRGAKAETPIEEFTPTPAFPALQYLESVDVEGVAWRAGLRTGDFLIEVNGVNVVKVGHKQVVGLIRQGGNRLVMKVVSVTRKPEEDGARRRAPPPPKRAPSTTLTLRSKSMTAELEELASIRSSEEEPELVFAVNLPPAQLSSSDEETREELARIGLVPPPEEFANGILLTTPPPGPGPLPTTVPSPASGKPSSELPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASTGLASAAGPARPRYLFQRRSKLWGDPVESRGLPGPEDDKPTVISELSSRLQQLNKDTRSLGEEPVGGLGSLLDPAKKSPIAAARLFSSLGELSTISAQRSPGGPGGGASYSVRPSGRYPVARRAPSPVKPASLERVEGLGAGVGGAGRPFGLTPPTILKSSSLSIPHEPKEVRFVVRSVSARSRSPSPSPLPSPSPGSGPSAGPRRPFQQKPLQLWSKFDVGDWLESIHLGEHRDRFEDHEIEGAHLPALTKEDFVELGVTRVGHRMNIERALRQLDGS

SEQ ID NO: 12 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 13 - protein, 587 aa, synthetic construct
MPKKKRKVCRSHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGDVNVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 14 - protein, 365 aa, synthetic construct
MPKKKRKVCRSHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRVNVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 15 - DNA, 5193 bp, Mus musculus
  source 1..5193: mol_type = genomic DNA
atggacggccccggggccagcgccgtggtcgtgcgcgtcggcatcccggacctgcaacaaacgaagtgcctgcgtctggacccaaccgcgcccgtgtgggccgccaagcagcgtgtgctctgcgccctcaatcatagccttcaagacgcgctcaactacgggctattccagcctccctcccggggtcgcgccggcaagttcctggatgaagagcggctcttacaggactacccgcctaacctggacacgcccctgccctatctggagttccgatacaagcggagagtttatgcccagaacctcatagatgacaagcagtttgcaaagctacacacaaaggcaaacctgaagaagttcatggactatgtccagctacacagcacagataaggtggcccgcctgctggacaaggggctggaccccaatttccatgaccctgactcaggagagtgccctctgagccttgcggcacagttggacaacgccactgacctcctgaaggttctccgcaacggcggtgctcatctggacttccggacccgagatgggctgacagccgtccactgtgctacccgccagcggaacgcaggggcattgacgaccctgctggacctgggggcttcgcctgactacaaggacagccgcggcctgacgcccctgtaccatagtgccctagggggcggggatgccctctgttgcgagctgcttctccatgatcatgcacagctggggaccactgatgagaatggttggcaagagatccatcaggcctgtcgctttggacacgtgcagcacctggagcaccttttgttctatggggccaacatgggtgctcagaatgcctcgggaaacacagccctgcacatctgtgccctctacaaccaggagagttgcgcgcgcgtcctgcttttccgtggtgccaacaaggacgtccgcaattacaacagccagacagccttccaggtggccattattgcagggaactttgagcttgccgaggtaatcaagacccacaaagactccgatgtcgtaccattcagggaaacccccagctatgcaaagcgacggcgtctggctggcccgagtggcctggcatccccacggcccttacagcgctcagccagtgatatcaacctgaaaggtgatcagcccgcagcttctccagggcccactctccgaagcctccctcatcaactcttgctccagaggcttcaggaggagaaagaccgtgacagggatggtgaactggagaatgacatcagcggcccctcagcaggcaggggtggccacaacaagatcagccccagtgggcccggcggatccggccccgcgcccggccccggcccggcgtctcccgcgccccccgcgccgccgccccggggcccgaagcggaaactttacagtgccgtccccggccgcaagttcatcgctgtgaaggcgcacagcccgcagggcgagggcgagatcccgctgcaccgcggcgaggccgtgaaggtgctcagcattggggagggcggtttctgggagggaaccgtgaagggccgaacaggctggttcccagctgactgtgtggaagaagtgcagatgcgacagtatgacacccggcatgaaaccagagaggaccggacgaagcgtctcttccgccactacactgtgggttcctatgacagcctcacttcacacagcgattatgtcatcgatgataaggtggctatcctgcagaaaagggaccatgaggggtttggctttgttctccggggagccaaagcagagacccccattgaggagtttacacccacacctgccttccctgcactccaataccttgagtctgtagatgtggaaggtgtggcctggagggctggacttcgaactggggacttcctcattgaggtgaacggagtgaatgtcgtgaaggttggacacaagcaagtggtgggtctcatccgtcagggtggcaaccgcctggtcatgaaggttgtgtctgtgaccaggaaacccgaggaggatggtgctcggcgcagagccccaccacccccaaagagggctcccagcaccacgctgaccctgcggtccaagtccatgacggctgagctcgaggaacttgcttccattcggagaagaaaaggggagaagttggatgagatcctggcagttgccgcggagccgacactgaggccggacattgcagatgctgactcgagggcggccactgtcaagcagcggcccaccagccggaggatcacccctgctgagatcagctcattgtttgagcgccagggcctcccaggcccagagaagctgccgggctctctgcggaaggggattccacggaccaaatctgtaggggaggatgagaagctggcatccctactggaagggcgcttcccacgcagcacgtcaatgcaggacacagtgcgtgaaggtcgaggcattccacccccgccgcagaccgccccgccacccccacccgcgccctactacttcgactccgggccaccccccaccttctcaccgccgccaccaccgggccgggcctatgacactgtgcgctccagcttcaaaccaggcctggaggctcgtctgggtgcgggggccgccggcctgtatgatccgagcacgcctctgggcccgctgccctaccctgagcgtcagaagcgtgcgcgctccatgatcatactgcaggactctgcgccagaagtgggtgatgtcccccggcctgcgcctgccgccacaccgcctgagcgccccaagcgccggcctcggccgtcaggccctgatagtccctatgccaacctgggcgccttcagtgccagcctcttcgctccgtcgaaaccacagcgccgcaagagcccgctggtgaagcagcttcaggtggaggacgctcaggagcgcgcggccctggccgtgggtagcccgggaccagtgggcggaagctttgcacgagaaccctctccgacgcaccgcgggccccggccaggcagccttgactacagctctggagaaggcctaggccttaccttcggcggccctagccctggcccagtcaaggagcggcgcctggaggagcgacgccgttccactgtgttcctctctgtgggtgccatcgagggcagccctcccagcgcggatctgccatccctacaaccctcccgctccattgatgagcgcctcctggggacaggcgccaccactggccgcgatttgctactcccctcccctgtctctgctctgaagccattggtcggtggtcccagccttgggccctcaggctccaccttcatccaccctctcactggcaaacccttggatcctagctcacccttagctcttgctctggctgcccgggagcgggctctggcctcgcaaacaccttcccggtcccccacacctgtgcacagccccgatgctgaccgccctggacccctctttgtggatgtgcaaacccgagactctgagagaggaccgttggcttccccagccttctcccctcggagtccagcgtggattccagtgcctgctcggagagaggcagagaagccccctcgggaagagcggaagtcaccagaggacaagaagtccatgatcctcagcgtcttggacacgtccttgcaacggccagctggcctcattgttgtgcatgccaccagcaatgggcaggagcccagcaggctgggggctgaagaggagcgccccggtactccggagctggccccagcccccatgcaggcagcagctgtggcagagcccatgccaagcccccgggcccagccccctggcagcatcccagcagatcccgggccaggtcaaggcagctcagaggaggagccagagctggtattcgctgtgaacctgccacctgctcagctgtcctccagcgatgaggagaccagagaggagctggcccgcatagggctagtgccaccccctgaagagtttgccaatgggatcctgctgaccaccccgcccccagggccgggccccttgcccaccacggtacccagcccggcctcagggaagcccagcagcgagctgccccctgcccctgagtctgcagctgactctggagtagaggaggctgacactcgaagctccagtgacccccacctggagaccacaagcaccatttccacagtgtccagcatgtccaccctgagctcggagagtggggaactcacggacacccacacctcctttgccgatggacacacttttctactcgagaagccaccagtgcctcccaagcccaagctcaagtccccgctggggaaggggccggtgaccttcagggacccgctgctgaagcaatcctcggacagtgagctcatggcccagcagcaccatgctgcctctactgggttggcttctgctgctgggcccgcccgccctcgctacctcttccagagaaggtccaagctgtggggggaccccgtggagagtcgggggctccctgggcctgaagatgacaaaccaactgtgatcagtgagctcagctcccgtctgcagcagctgaataaagacacacgctccttgggggaggaaccagttggtggcctgggcagcctgctggaccctgctaagaagtcacccattgcagcagctcggctcttcagcagcctcggtgagctgagcaccatctcagcgcagcgcagcccggggggcccgggcggaggggcctcctactcggtgcggcccagcggccggtaccccgtggcgagacgagccccgagcccagtgaaacccgcatcgctggagcgggtggaggggctgggggcgggcgtgggaggcgcggggcggcccttcggcctcacgcctcccaccatcctcaagtcgtccagcctctccatcccgcacgaacccaaggaagtgcgcttcgtggtgcgaagtgtgagtgcgcgcagccgctccccctcaccatctccgctgccctcgccttctcccggctctggccccagtgccggcccgcgtcggccatttcaacagaagcccctgcagctctggagcaagttcgatgtgggcgactggctggagagcatccacttaggcgagcaccgagaccgcttcgaggaccatgagatcgaaggcgcacacctgcctgcgctcaccaaggaagacttcgtggagctgggcgtcacacgcgttggccaccgcatgaacatcgagcgtgcgctcaggcagctggatggcagctga

SEQ ID NO: 16 - DNA, 5421 bp, Homo sapiens
  source 1..5421: mol_type = genomic DNA
atgcagctgagccgcgccgccgccgccgccgccgccgcccctgcggagcccccggagccgctgtcccccgcgccggccccggccccggccccccccggccccctcccgcgcagcgcggccgacggggctccggcgggggggaagggggggccggggcgccgcgcggagtccccgggcgctccgttccccggcgcgagcggccccggcccgggccccggcgcggggatggacggccccggggccagcgccgtggtcgtgcgcgtcggcatcccggacctgcagcagacgaagtgcctgcgcctggacccggccgcgcccgtgtgggccgccaagcagcgcgtgctctgcgccctcaaccacagcctccaggacgcgctcaactatgggcttttccagccgccctcccggggccgcgccggcaagttcctggatgaggagcggctcctgcaggagtacccgcccaacctggacacgcccctgccctacctggagtttcgatacaagcggcgagtttatgcccagaacctcatcgatgataagcagtttgcaaagcttcacacaaaggcgaacctgaagaagttcatggactacgtccagctgcatagcacggacaaggtggcacgcctgttggacaaggggctggaccccaacttccatgaccctgactcaggagagtgccccctgagcctcgcagcccagctggacaacgccacggacctgctaaaggtgctgaagaatggtggtgcccacctggacttccgcactcgcgatgggctcactgccgtgcactgtgccacacgccagcggaatgcggcagcactgacgaccctgctggacctgggggcttcacctgactacaaggacagccgcggcttgacacccctctaccacagcgccctggggggtggggatgccctctgctgtgagctgcttctccacgaccacgctcagctggggatcaccgacgagaatggctggcaggagatccaccaggcctgccgctttgggcacgtgcagcatctggagcacctgctgttctatggggcagacatgggggcccagaacgcctcggggaacacagccctgcacatctgtgccctctacaaccaggagagctgtgctcgtgtcctgctcttccgtggagctaacagggatgtccgcaactacaacagccagacagccttccaggtggccatcatcgcagggaactttgagcttgcagaggttatcaagacccacaaagactcggatgttgtaccattcagggaaacccccagctatgcgaagcggcggcgactggctggccccagtggcttggcatcccctcggcctctgcagcgctcagccagcgatatcaacctgaagggggaggcacagccagcagcttctcctggaccctcgctgagaagcctcccccaccagctgctgctccagcggctgcaagaggagaaagatcgtgaccgggatgccgaccaggagagcaacatcagtggccctttagcaggcagggccggccaaagcaagatcagcccgagcgggcccggcggccccggccccgcgcccggccccggccccgcgccccctgcgccccccgcaccgccgccccggggcccgaagcggaaactttacagcgccgtccccggccgcaagttcatcgccgtgaaggcgcacagcccgcagggtgaaggcgagatcccgctgcaccgcggcgaggccgtgaaggtgctcagcattggggagggcggtttctgggagggaaccgtgaaaggccgcacgggctggttcccggccgactgcgtggaggaagtgcagatgaggcagcatgacacacggcctgaaacgcgggaggaccggacgaagcggctctttcggcactacacagtgggctcctacgacagcctcacctcacacagcgattatgtcattgatgacaaagtggctgtcctgcagaaacgggaccacgagggctttggttttgtgctccggggagccaaagcagagacccccatcgaggagttcacgcccacgccagccttcccggcgctgcagtatctcgagtcggtggacgtggagggtgtggcctggagggccgggctgcgcacgggagacttcctcatcgaggtgaacggggtgaacgtggtgaaggtcggacacaagcaggtggtggctctgattcgccagggtggcaaccgcctcgtcatgaaggttgtgtctgtgacaaggaagccagaagaggacggggctcggcgcagagccccaccgccccccaagagggcccccagcaccacactgaccctgcgctccaagtccatgacagctgagctcgaggaacttgcctccattcggagaagaaaaggggagaagctggacgagatgctggcagccgccgcagagccaacgctgcggccagacatcgcagacgcagactccagagccgccaccgtcaaacagaggcccaccagtcggaggatcacacccgccgagattagctcattgtttgaacgccagggcctcccaggcccagagaagctgccgggctccttgcggaaggggattccacggaccaagtctgtaggggaggacgagaagctggcgtccctgctggaagggcgcttcccgcggagcacctcgatgcaagacccggtgcgcgagggtcgcggcatcccgcccccgccgcagaccgcgccgcctcccccgcccgcgccctactacttcgactcggggccgcccccggccttctcgccgccgcccccgccgggccgcgcctacgacacggtgcgctccagcttcaagcccggcctggaggcgcgcctgggcgcgggcgctgccggcctgtacgagccgggcgcggccctcggcccgctgccgtatcccgagcggcagaagcgcgcgcgctccatgatcatcctgcaggactcggcgcccgagtcgggcgacgcccctcgacccccgcccgcggccaccccgcccgagcgacccaagcgccggccgcggccgcccggccccgacagcccctacgccaacctgggcgccttcagcgccagcctcttcgctccgtccaagccgcagcgccgcaagagccccctggtgaagcagctgcaggtggaggacgcgcaggagcgcgcggccctggccgtgggcagccccggtcccggcggcggcagcttcgcccgcgagccctccccgacccaccgcggtccgcgcccgggtggcctcgactacggcgcgggcgatggcccggggctcgcgttcggcggcccgggcccggccaaggaccggcggctggaggagcggcgccgctccactgtgttcctgtccgtgggggccatcgagggcagcgcccccggcgcggatctgccatccctacagccctcccgctccatcgacgagcgcctcctggggaccggccccaccgccggccgcgacctgctgctgccctccccggtgtctgccctgaagccgttggtcagcggcccgagcctggggccctcgggttccaccttcatccacccactcaccggcaaacccctggaccccagctcacccctggcccttgccctggctgcccgagagcgagctctggcctcccaggcgccctcccggtcccccacacccgtgcacagtcccgacgccgaccgccccggacccctgtttgtggatgtacaggcccgggacccagagcgagggtccctggcttccccggctttctccccacggagcccagcctggattcctgtgcctgctcgcagggaggcagagaaggtcccccgggaggagcggaagtcacccgaggacaagaagtccatgatcctcagcgtcctggacacatccctgcagcggccagctggcctcatcgttgtgcacgccaccagcaacgggcaggagcccagcaggctggggggggccgaagaggagcgcccgggcaccccggagttggccccggcccccatgcagtcagcggctgtggcagagcccctgcccagcccccgggcccagccccctggtggcaccccggcagacgccgggccaggccagggcagctcagaggaagagccagagctggtgtttgctgtgaacctgccacctgcccagctgtcgtccagcgatgaggagaccagggaggagctggcccgaattgggttggtgccaccccctgaagagtttgccaacggggtcctgctggccaccccactcgctggcccgggcccctcgcccaccacggtgcccagcccggcctcagggaagcccagcagtgagccaccccctgcccctgagtctgcagccgactctggggtggaggaggctgacacacgcagctccagcgacccccacctggagaccacaagcaccatctccacggtgtccagcatgtccaccttgagctcggagagcggggaactcactgacacccacacctccttcgctgacggacacacttttctactcgagaagccaccagtgcctcccaagcccaagctcaagtccccgctggggaaggggccggtgaccttcagggacccgctgctgaagcagtcctcggacagcgagctcatggcccagcagcaccacgccgcctctgccgggctggcctctgccgccgggcctgcccgccctcgctacctcttccagagaaggtccaagctatggggggaccccgtggagagccgggggctccctgggcctgaagacgacaaaccaactgtgatcagtgagctcagctcccgcctgcagcagctgaacaaggacacgcgttccctgggggaggaaccagttggtggcctgggcagcctgctggaccctgccaagaagtcgcccatcgcagcagctcggctcttcagcagcctcggtgagctgagctccatttcagcgcagcgcagccccgggggcccgggcggcggggcctcgtactcggtgaggcccagtggccgctaccccgtggcgagacgcgccccgagcccggtgaagcccgcgtcgctggagcgggtggaggggctgggggcgggcgcggggggcgcagggcggcccttcggcctcacgccccccaccatcctcaagtcgtccagcctctccatcccgcacgagcccaaggaggtgcgcttcgtggtgcgcagcgtgagcgcgcgcagtcgctccccctcgccgtcgccgctgccctcgcccgcgtccggccccggccccggcgcccccggcccacgccgacccttccagcagaagccgctgcagctctggagcaagttcgacgtgggcgactggctggagagcatccacctaggcgagcaccgcgaccgcttcgaggaccatgagatagaaggcgcgcacctacccgcgcttaccaaggacgacttcgtggagctgggcgtcacgcgcgtgggccaccgcatgaacatcgagcgcgcgctcaggcagctggacggcagctga

SEQ ID NO: 17 - protein, 706 aa, Mus musculus
MGPKRKLYSAVPGRKFIAVKAHSPQGEGEIPLHRGEAVKVLSIGEGGFWEGTVKGRTGWFPADCVEEVQMRQYDTRHETREDRTKRLFRHYTVGSYDSLTSHSDYVIDDKVAILQKRDHEGFGFVLRGAKAETPIEEFTPTPAFPALQYLESVDVEGVAWRAGLRTGDFLIEVNGVNVVKVGHKQVVGLIRQGGNRLVMKVVSVTRKPEEDGARRRAPPPPKRAPSTTLTLRSKSMTAELEELASIRSSEEEPELVFAVNLPPAQLSSSDEETREELARIGLVPPPEEFANGILLTTPPPGPGPLPTTVPSPASGKPSSELPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASTGLASAAGPARPRYLFQRRSKLWGDPVESRGLPGPEDDKPTVISELSSRLQQLNKDTRSLGEEPVGGLGSLLDPAKKSPIAAARLFSSLGELSTISAQRSPGGPGGGASYSVRPSGRYPVARRAPSPVKPASLERVEGLGAGVGGAGRPFGLTPPTILKSSSLSIPHEPKEVRFVVRSVSARSRSPSPSPLPSPSPGSGPSAGPRRPFQQKPLQLWSKFDVGDWLESIHLGEHRDRFEDHEIEGAHLPALTKEDFVELGVTRVGHRMNIERALRQLDGS

SEQ ID NO: 18 - protein, 707 aa, Homo sapiens
MGPKRKLYSAVPGRKFIAVKAHSPQGEGEIPLHRGEAVKVLSIGEGGFWEGTVKGRTGWFPADCVEEVQMRQHDTRPETREDRTKRLFRHYTVGSYDSLTSHSDYVIDDKVAVLQKRDHEGFGFVLRGAKAETPIEEFTPTPAFPALQYLESVDVEGVAWRAGLRTGDFLIEVNGVNVVKVGHKQVVALIRQGGNRLVMKVVSVTRKPEEDGARRRAPPPPKRAPSTTLTLRSKSMTAELEELASIRSSEEEPELVFAVNLPPAQLSSSDEETREELARIGLVPPPEEFANGVLLATPLAGPGPSPTTVPSPASGKPSSEPPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASAGLASAAGPARPRYLFQRRSKLWGDPVESRGLPGPEDDKPTVISELSSRLQQLNKDTRSLGEEPVGGLGSLLDPAKKSPIAAARLFSSLGELSSISAQRSPGGPGGGASYSVRPSGRYPVARRAPSPVKPASLERVEGLGAGAGGAGRPFGLTPPTILKSSSLSIPHEPKEVRFVVRSVSARSRSPSPSPLPSPASGPGPGAPGPRRPFQQKPLQLWSKFDVGDWLESIHLGEHRDRFEDHEIEGAHLPALTKDDFVELGVTRVGHRMNIERALRQLDGS

SEQ ID NO: 19 - protein, 1091 aa, Mus musculus
MDGPGASAVVVRVGIPDLQQTKCLRLDPTAPVWAAKQRVLCALNHSLQDALNYGLFQPPSRGRAGKFLDEERLLQDYPPNLDTPLPYLEFRYKRRVYAQNLIDDKQFAKLHTKANLKKFMDYVQLHSTDKVARLLDKGLDPNFHDPDSGECPLSLAAQLDNATDLLKVLRNGGAHLDFRTRDGLTAVHCATRQRNAGALTTLLDLGASPDYKDSRGLTPLYHSALGGGDALCCELLLHDHAQLGTTDENGWQEIHQACRFGHVQHLEHLLFYGANMGAQNASGNTALHICALYNQESCARVLLFRGANKDVRNYNSQTAFQVAIIAGNFELAEVIKTHKDSDVVPFRETPSYAKRRRLAGPSGLASPRPLQRSASDINLKGAPPPRGPKRKLYSAVPGRKFIAVKAHSPQGEGEIPLHRGEAVKVLSIGEGGFWEGTVKGRTGWFPADCVEEVQMRQYDTRHETREDRTKRLFRHYTVGSYDSLTSHSDYVIDDKVAILQKRDHEGFGFVLRGAKAETPIEEFTPTPAFPALQYLESVDVEGVAWRAGLRTGDFLIEVNGVNVVKVGHKQVVGLIRQGGNRLVMKVVSVTRKPEEDGARRRAPPPPKRAPSTTLTLRSKSMTAELEELASIRSSEEEPELVFAVNLPPAQLSSSDEETREELARIGLVPPPEEFANGILLTTPPPGPGPLPTTVPSPASGKPSSELPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASTGLASAAGPARPRYLFQRRSKLWGDPVESRGLPGPEDDKPTVISELSSRLQQLNKDTRSLGEEPVGGLGSLLDPAKKSPIAAARLFSSLGELSTISAQRSPGGPGGGASYSVRPSGRYPVARRAPSPVKPASLERVEGLGAGVGGAGRPFGLTPPTILKSSSLSIPHEPKEVRFVVRSVSARSRSPSPSPLPSPSPGSGPSAGPRRPFQQKPLQLWSKFDVGDWLESIHLGEHRDRFEDHEIEGAHLPALTKEDFVELGVTRVGHRMNIERALRQLDGS

SEQ ID NO: 20 - protein, 1092 aa, Homo sapiens
MDGPGASAVVVRVGIPDLQQTKCLRLDPAAPVWAAKQRVLCALNHSLQDALNYGLFQPPSRGRAGKFLDEERLLQEYPPNLDTPLPYLEFRYKRRVYAQNLIDDKQFAKLHTKANLKKFMDYVQLHSTDKVARLLDKGLDPNFHDPDSGECPLSLAAQLDNATDLLKVLKNGGAHLDFRTRDGLTAVHCATRQRNAAALTTLLDLGASPDYKDSRGLTPLYHSALGGGDALCCELLLHDHAQLGITDENGWQEIHQACRFGHVQHLEHLLFYGADMGAQNASGNTALHICALYNQESCARVLLFRGANRDVRNYNSQTAFQVAIIAGNFELAEVIKTHKDSDVVPFRETPSYAKRRRLAGPSGLASPRPLQRSASDINLKGAPPPRGPKRKLYSAVPGRKFIAVKAHSPQGEGEIPLHRGEAVKVLSIGEGGFWEGTVKGRTGWFPADCVEEVQMRQHDTRPETREDRTKRLFRHYTVGSYDSLTSHSDYVIDDKVAVLQKRDHEGFGFVLRGAKAETPIEEFTPTPAFPALQYLESVDVEGVAWRAGLRTGDFLIEVNGVNVVKVGHKQVVALIRQGGNRLVMKVVSVTRKPEEDGARRRAPPPPKRAPSTTLTLRSKSMTAELEELASIRSSEEEPELVFAVNLPPAQLSSSDEETREELARIGLVPPPEEFANGVLLATPLAGPGPSPTTVPSPASGKPSSEPPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASAGLASAAGPARPRYLFQRRSKLWGDPVESRGLPGPEDDKPTVISELSSRLQQLNKDTRSLGEEPVGGLGSLLDPAKKSPIAAARLFSSLGELSSISAQRSPGGPGGGASYSVRPSGRYPVARRAPSPVKPASLERVEGLGAGAGGAGRPFGLTPPTILKSSSLSIPHEPKEVRFVVRSVSARSRSPSPSPLPSPASGPGPGAPGPRRPFQQKPLQLWSKFDVGDWLESIHLGEHRDRFEDHEIEGAHLPALTKDDFVELGVTRVGHRMNIERALRQLDGS

SEQ ID NO: 21 - DNA, 6238 bp, synthetic construct
  source 1..6238: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaattaagtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagaggatccttcgaaaccggtgctagcaccatgggcccgaagcggaaactttacagcgccgtccccggccgcaagttcatcgccgtgaaggcgcacagcccgcagggtgaaggcgagatcccgctgcaccgcggcgaggccgtgaaggtgctcagcattggggagggcggtttctgggagggaaccgtgaaaggccgcacgggctggttcccggccgactgcgtggaggaagtgcagatgaggcagcatgacacacggcctgaaacgcgggaggaccggacgaagcggctctttcggcactacacagtgggctcctacgacagcctcacctcacacagcgattatgtcattgatgacaaagtggctgtcctgcagaaacgggaccacgagggctttggttttgtgctccggggagccaaagcagagacccccatcgaggagttcacgcccacgccagccttcccggcgctgcagtatctcgagtcggtggacgtggagggtgtggcctggagggccgggctgcgcacgggagacttcctcatcgaggtgaacggggtgaacgtggtgaaggtcggacacaagcaggtggtggctctgattcgccagggtggcaaccgcctcgtcatgaaggttgtgtctgtgacaaggaagccagaagaggacggggctcggcgcagagccccaccgccccccaagagggcccccagcaccacactgaccctgcgctccaagtccatgacagctgagctcgaggaacttgcctccattcggagctcagaggaagagccagagctggtgtttgctgtgaacctgccacctgcccagctgtcgtccagcgatgaggagaccagggaggagctggcccgaattgggttggtgccaccccctgaagagtttgccaacggggtcctgctggccaccccactcgctggcccgggcccctcgcccaccacggtgcccagcccggcctcagggaagcccagcagtgagccaccccctgcccctgagtctgcagccgactctggggtggaggaggctgacacacgcagctccagcgacccccacctggagaccacaagcaccatctccacggtgtccagcatgtccaccttgagctcggagagcggggaactcactgacacccacacctccttcgctgacggacacacttttctactcgagaagccaccagtgcctcccaagcccaagctcaagtccccgctggggaaggggccggtgaccttcagggacccgctgctgaagcagtcctcggacagcgagctcatggcccagcagcaccacgccgcctctgccgggctggcctctgccgccgggcctgcccgccctcgctacctcttccagagaaggtccaagctatggggggaccccgtggagagccgggggctccctgggcctgaagacgacaaaccaactgtgatcagtgagctcagctcccgcctgcagcagctgaacaaggacacgcgttccctgggggaggaaccagttggtggcctgggcagcctgctggaccctgccaagaagtcgcccatcgcagcagctcggctcttcagcagcctcggtgagctgagctccatttcagcgcagcgcagccccgggggcccgggcggcggggcctcgtactcggtgaggcccagtggccgctaccccgtggcgagacgcgccccgagcccggtgaagcccgcgtcgctggagcgggtggaggggctgggggcgggcgcggggggcgcagggcggcccttcggcctcacgccccccaccatcctcaagtcgtccagcctctccatcccgcacgagcccaaggaggtgcgcttcgtggtgcgcagcgtgagcgcgcgcagtcgctccccctcgccgtcgccgctgccctcgcccgcgtccggccccggccccggcgcccccggcccacgccgacccttccagcagaagccgctgcagctctggagcaagttcgacgtgggcgactggctggagagcatccacctaggcgagcaccgcgaccgcttcgaggaccatgagatagaaggcgcgcacctacccgcgcttaccaaggacgacttcgtggagctgggcgtcacgcgcgtgggccaccgcatgaacatcgagcgcgcgctcaggcagctggacggcagctgacggcgcgccaccggtcaaacaccattgtcacactccatactatcaaacaccattgtcacactccagatcaacaaacaccattgtcacactccacgccggcgggtaccagtgaattctaccagtgccatagatcaaagtgaattctaccagtgccatatactatagtgaattctaccagtgccatacaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 22 - DNA, 495 bp, Homo sapiens
  source 1..495: mol_type = genomic DNA
gtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctaga